(19) United States
(12) Patent Application Publication
(10) Pub. No.: US 2011/0269953 A1
(43) Pub. Date: Nov. 3, 2011

(54) Nitrogen and Sulfur-Containing Heterocycle Derivatives

(76) Inventor: Murty N. Armilli, Oak Ridge, NC (US)

(21) Appl. No.: 13/037,358

(22) Filed: Mar. 1, 2011

Related U.S. Application Data
(63) Continuation of application No. PCT/US2009/055464, filed on Aug. 29, 2009.
(60) Provisional application No. 61/092,764, filed on Aug. 29, 2008.

(51) Int. Cl. C07D 413/14; C07D 417/14; C07D 279/12; C07D 413/06

(52) U.S. Cl. 544/58.2

(57) ABSTRACT

The present invention provides nitrogen and sulfur-containing heterocycle compounds to be used as scaffolds and, in particular, nitrogen and sulfur-containing heterocycle compounds having a thiomorpholine core. The compounds herein described may be useful in treating diseases such as diabetes, obesity, cancer, cardiovascular, Alzheimer's, inflammatory, antidepressant, rheumatoid arthritis, multiple sclerosis, allergic rhinitis, asthma as well as viral and bacterial infections. The compounds herein described may also be useful in treating CNS disorders such as but not limited to Schizophrenia, Alzheimer's disease (AD).

### BACKGROUND OF THE INVENTION

The present invention relates generally to nitrogen and sulfur-containing heterocycle compounds to be used as scaffolds and, in particular, to nitrogen and sulfur-containing heterocycle compounds having a thiomorpholine core.

Drug like scaffolds are desirable in drug discovery as they allow for the production of a large number of compounds based on a common core structure. There are several scaffolds currently utilized in the field of drug discovery, allowing for the production of molecules having the best fit with their target binding sites. Examples such as rhodanines, oxazolidinones and hydantions, showed promise in their respective areas. Synthesis of several analogs on these five-membered ring scaffolds has been well documented in the field. However, not much work has been done in six membered thiomorpholines where the scaffold can be amenable to synthesis of a great number of diverse analogs.

Therefore, it would be desirable to have a six-membered ring scaffold based on a thiomorpholine core.

### SUMMARY OF THE INVENTION

Broadly, the present invention provides nitrogen and sulfur-containing heterocycle compounds to be used as scaffolds and, in particular, nitrogen and sulfur-containing heterocycle compounds having a thiomorpholine core. The compounds herein described may be useful in treating diseases such as diabetes, obesity, cancer, cardiovascular, Alzheimer's, inflammatory, antidepressant, rheumatoid arthritis, multiple sclerosis, allergic rhinitis, asthma as well as viral and bacterial infections. The compounds herein described may also be useful in treating CNS disorders such as but not limited to Schizophrenia, Alzheimer's disease (AD).

These and other features, aspects and advantages of the present invention will become better understood with reference to the following drawings, description and claims.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides nitrogen and sulfur-containing heterocycle compounds to be used as scaffolds and, in particular, nitrogen and sulfur-containing heterocycle compounds having a thiomorpholine core. The compounds herein described may be useful in treating diseases such as diabetes, obesity, cancer, cardiovascular, Alzheimer's, inflammatory, antidepressant, rheumatoid arthritis, multiple sclerosis, allergic rhinitis, asthma as well as viral and bacterial infections. The compounds herein described may also be useful in treating CNS disorders such as but not limited to Schizophrenia, Alzheimer's disease (AD).

In one embodiment of the present invention there is provided a compound (I) comprising the formula:

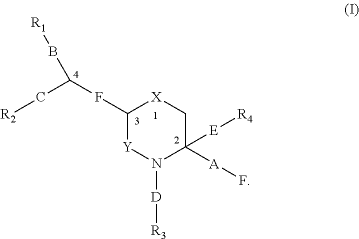

wherein:

X is —S—, —S(O)—, or —S(O)₂—;

Y is —CH₂—,

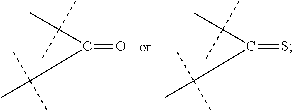

A is —(CH₂)ₘ— where m=0, 1, 2, 3, or 4, —(CH═CR₅)ₙ— where R₅ is a hydrogen, an alkyl, a cycloalkyl, a heterocyclyl, an aryl or an heteroaryl and n=0, 1, or 2, -alkenylene-, -alkynylene-, -cycloalkylene-, -heterocyclylene-, -arylene-, -fused heterocyclylarylene-, -fused heterocyclylheterocyclylene-, -fused heterocyclylheteroarylene-, -fused heterocyclylcycloalkylene-, -fused heteroarylarylene-, -fused heteroarylheterocyclylene-, -fused heteroarylheteroarylene-, or -fused heteroarylcycloalkylene-;

R is —C(O)R₆, —OR₇, —NR₈R₉, —SR₁₀, —S(O)R₁₁, —S(O)₂R₁₂, —S(O)₂NHC(O)-alkyl, —S(O)₂NHC(O)-aryl, —S(O)₂NHC(O)-heteroaryl, —S(O)₂NHC(O)-alkylenearyl, —S(O)₂NHC(O)-alkyleneheteroryl, —S(O)₂NHC(O)-arylenealkyl, —CHR₁₃R₁₄, —CN, -J, -alkylene-J, -arylene-J, -cycloalkylene-J, -alkyleneheterocyclylene-J, -alkenylheterocyclylene-J, -alkynyleneheterocyclylene-J, -alkyleneheteroarylene-J, -alkenylheteroarylene-J, —NHCH2-J, —NR₁₃CHR₁₄-J, —NHS(O)₂-alkyl, —NHS(O)₂-aryl, —NHS(O)₂-heteroaryl, —NHS(O)₂-cycloalkyl, —NHS(O)₂-fused heteroaryl, —NHS(O)₂-alkylene-J, —NHS(O)₂-arylene-J, —NHS(O)₂-heteroarylene-J, —NHS(O)₂-cycloalkylene-J, —NHS(O)₂-fused heteroarylene-J, —P(O)(OH)(O-alkyl), or —P(O)(O-alkyl)₂;

wherein J is —H; —OH; —COOH; —P(O)(OH)₂; —S(O)₂OH; —B(OH)₂; -acid isostere;

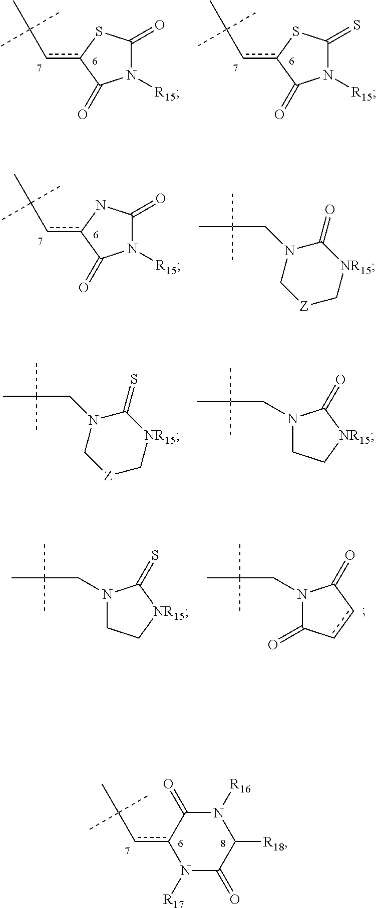

wherein Z is —CR₁₃R₁₄—; —O—; —NR₁₅—; —S—; —S(O)—; or —S(O)₂—; and wherein the stereocenters 6 & 7 may possess an E, Z or EZ configuration and stereocenter 8 may possess an R, S or RS configuration.

wherein R₁₅, R₁₆, and R₁₇ are each independently —H; -alkyl; -cycloalkyl; -aryl; -alkylenearyl; -alkenylenearyl; -alkynylenearyl; -heterocyclyl; -heteroaryl; -alkyleneheterocyclyl; -alkenyleneheterocyclyl; -alkynyleneheterocyclyl; -alkyleneheteroaryl; -alkenyleneheteroaryl; or -alkynyleneheteroaryl and R₁₈ is —H; -alkyl; -cycloalkyl; -aryl; -heterocyclyl; -heteroaryl; -alkylenecycloalkyl; -alkylenearyl; -alkyleneheterocyclyl; or -alkyleneheteroaryl;

wherein R₆ is —H; —OR₁₉; —CHR₂₀R₂₁; —NR₂₂R₂₃; —NHS(O)₂-alkyl; —NHS(O)₂-aryl; —NHS(O)₂-heteroaryl; —NHS(O)₂-heterocyclyl; —NHS(O)₂-alkylenearyl; —NHS(O)₂-alkyleneheteroaryl; —NHS(O)₂-alkyleneheterocyclyl; —NHS(O)₂-arylenealkyl; -cycloalkyl: -heterocyclyl; -aryl; -aryloxyalkyl; -cycloalkylaryl; -heteroaryl; -alkenyleneheteroaryl; -alkynyleneheteroaryl; -alkenylenearyl; -alkynylenearyl; -fused cycloalkyl; -fused aryl; -fused heteroaryl; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; or -fused heteroarylheterocyclyl;

wherein R₁₉ is —H; -alkyl; -cycloalkyl; -perhaloalkyl; -heterocyclyl; -aryl; -heteroaryl; -alkylene-heteroaryl; -alkylene-aryl; or -arylene-alkyl;

and wherein R₂₀ and R₂₁ are each independently —H; -alkyl; -cycloalkyl; -aryl; -alkylenearyl; -alkenylenearyl; -alkynylenearyl; -heterocyclyl; -alkyleneheterocyclyl; -alkenyleneheterocyclyl; -alkynyleneheterocyclyl; -heteroaryl; -alkyleneheteroaryl; -alkenyleneheteroaryl; or -alkynyleneheteroaryl;

and wherein R₂₂ and R₂₃ are each independently: —H; —C(O)CR₂₉R₃₀NH[C(O)CR₂₉R₃₀NH]nR₃₁; wherein n=0, 1, 2, or 3; —C(O)CH2CR₂₉R₃₀NH[C(O)CHR₂₉NH]nR₃₁; wherein n=0, 1, 2, or 3; —C(O)CR₂₉R₃₀NH[C(O)CH2CHR₂₉NH]nR₃₁; wherein n=0, 1, 2, or 3; -alkyl-J; -cycloalkyl-J; -aryl-J; -alkylenearyl-J; -alkenylenearyl-J; -alkynylenearyl-J; -heterocyclyl-J; -alkyleneheterocyclyl-J; -alkenyleneheterocyclyl-J; -alkynyleneheterocyclyl-J; -aryloxyalkyl-J; -alkoxyaryl-J; -heteroaryl-J; -alkyleneheteroaryl-J; -alkenyleneheteroaryl-J; -alkynyleneheteroaryl-J; -fused cycloalkyl-J; -fused aryl-J; -fused heteroaryl-J; -fused cycloalkylaryl-J; -fused arylcycloalkyl-J; -fused heterocyclylaryl-J; -fused arylheterocyclyl-J; -fused cycloalkylheteroaryl-J; -fused heteroarylcycloalkyl-J; -fused heterocyclylheteroaryl-J; or -fused heteroarylheterocyclyl-J; and wherein R₂₂ and R₂₃ together may form a ring having the formula —(CH₂)ₐ-M-(CH₂)_{b}— bonded to the nitrogen atom to which R₂₂ and R₂₃ are attached and wherein a and b are independently 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—;

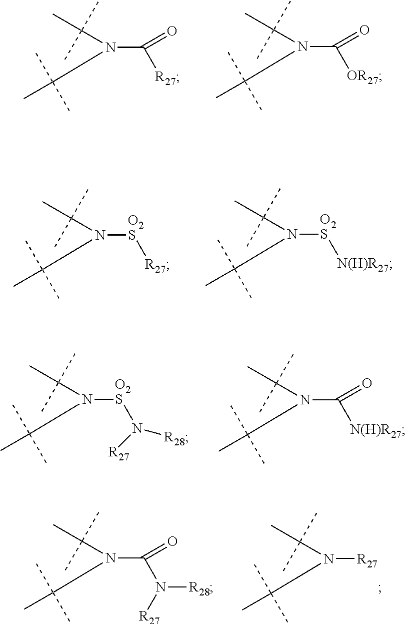

wherein R₂₇ and R₂₈ are each independently —H; —CN; —NO₂; -alkyl; -cycloalkyl; -heterocyclyl; -aryl; -heteroaryl; —C(O)—O-alkyl; —C(O)—O-aryl; —C(O)—O-alkylenearyl; -alkylene-heterocyclyl; -alkylene-cycloalkyl; -alkylene-aryl; or -alkylene-heteroaryl;

wherein R₂₄ is —H; —C(O)R₆; —SR₁₀; —S(O)R₁₁; —S(O)₂R₁₂; —S(O)₂NHC(O)-alkyl; —S(O)₂NHC(O)-aryl; —S(O)₂NHC(O)-heteroaryl; —S(O)₂NHC(O)-alkylenearyl; —S(O)₂NHC(O)-alkyleneheteroryl; —S(O)₂NHC(O)-arylenealkyl; an acid isostere; —CN; —P(O)(OH)(O-alkyl); —P(O)(O-alkyl)₂; —P(O)(OH)₂; —C(O)OH; or -acid isostere;

wherein R₂₅ and R₂₆ are each independently —H; -alkyl; cycloalkyl; -aryl; -alkylenearyl; -alkenylenearyl; -alkynylenearyl; -heterocyclyl; -alkyleneheterocyclyl; -alkenyleneheterocyclyl; or -alkynyleneheterocyclyl; and wherein R₂₅ and R₂₆ together may form a ring having the formula —(CH₂)ₐ-M-(CH₂)_{b}— bonded to the nitrogen atom to which R₂₂ and R₂₃ are attached wherein a and b are independently equal to 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—;

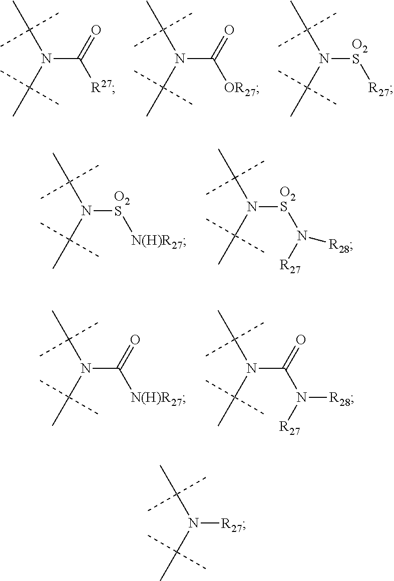

wherein R₂₉, R₃₀ and R₃₁ are each independently —H; -alkyl; -cycloalkyl; -aryl; -heterocyclyl; -heteroaryl; -alkylenecycloalkyl; -alkylenearyl-J; -alkyleneheteroaryl; or -alkylene-J;

wherein R₇ is: —H; -alkyl; -cycloalkyl: -heterocyclyl; -aryl; -aryloxy; -alkoxy; -heteroaryloxy; -heteroaryl; -alkyleneheteroaryl; -alkenyleneheteroaryl; -alkynyleneheteroaryl; -alkylenearyl; -alkenylenearyl; -alkynylenearyl; -perhaloalkyl; —alkylene-T-R₂₄; -cycloalkylene-T-R₂₄; -heterocyclylene-T-R₂₄; -arylene-T-R₂₄; -heteroarylene-T-R₂₄; -alkylene-C(O)NR₂₅R₂₆; -alkylene-NR₂₅R₂₆; -fused cycloalkyl; -fused aryl; -fused heteroaryl; -fused cycloalkylaryl; -fused arylcycloalkyl; -fused heterocyclylaryl; -fused arylheterocyclyl; -fused heteroarylcycloalkyl; -fused heterocyclylheteroaryl; or -fused heteroarylheterocyclyl; wherein T is alkylene; arylene; heteroarylene; —(CH₂)_{d}—, d=0 or 1; —O—; —N(R₂₇)—; —S—; —S(O)—; —S(O)₂—; —O—S(O)—; and —O—C(O)—; —C(O)—O—; —N(R₂₇)C(O)—; —C(O)N(R₂₇)—; —N(R₂₇)C(O)N(R₂₈)—; —N(R₂₇)S(O)₂—; —S(O)₂N(R₂₇)—; —N(R₂₇)S(O)₂N(R₂₈)—; —C(O)N(R₂₇)S(O)₂—; —N(R₂₇)C(O)—O—; —O—C(O)N(R₂₇)—; —N═N—; —N(R₂₇)—N(R₂₈)—;

wherein R₈ and R₉ are each independently: —H; -alkyl; -cycloalkyl; -aryl; -alkylenearyl; -alkenylenearyl; -alkynylenearyl; -heterocyclyl; -heteroaryl; -alkyleneheteroaryl; -alkenyleneheteroaryl; -alkynyleneheteroaryl; -alkyleneheterocyclyl; -alkenyleneheterocyclyl; -alkynyleneheterocyclyl; -heterocyclylalkyl; -heterocyclylaryl; -fused aryl; -fused cycloalkylaryl; -fused arylcycloalkyl; -fused cycloalkylheteroaryl; -fused heteroarylcycloalkyl; -fused heteroaryl; -fused cycloalkylheteroaryl; -fused heteroarylcycloalkyl; —S(O)₂R₃₂; —C(O)R₃₂; —C(O)NR₃₃R₃₄; —S(O)₂NR₃₃R₃₄; —C(O)CR₂₉R₃₀NH[C(O)CHR₂₉NH]nR₃₁, wherein n=0, 1, 2, or 3; —C(O)CH2CR₂₉R₃₀NH[C(O)CHR₂₉NH]nR₃₁ wherein n=0, 1, 2, or 3; —C(O)CR₂₉R₃₀NH[C(O)CH2CHR₂₉NH]nR₃₁ wherein n=0, 1, 2, or 3; -alkylene-J; -alkenylene-J; -alkynylene-J; or -arylene-J; wherein R₈ and R₉ together may form a ring having the formula —(CH₂)ₒ-M-(CH₂)ₚ— bonded to the nitrogen atom to which R₈ and R₉ are attached wherein o and p are independently equal to 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—;

wherein R₃₂ is -alkyl; -alkenylenealkyl; -alkynylenealkyl; -cycloalkyl; -alkylenecycloalkyl; -aryl; -alkylenearyl; -alkenylenearyl; -alkynylenearyl; -heterocyclyl; -alkyleneheterocyclyl; -alkenyleneheterocyclyl; -alkynyleneheterocyclyl; -heteroaryl; -alkyleneheteroaryl; -alkenyleneheteroaryl; -alkynyleneheteroaryl; -fused cycloalkyl; -fused aryl; -fused heteroaryl; -fused cycloalkylaryl; -fused arylcycloalkyl; -fused heterocyclylaryl; -fused arylheterocyclyl; -fused heteroarylcycloalkyl; -fused heterocyclylheteroaryl; -fused heteroarylheterocyclyl;

and wherein R₃₃ and R₃₄ are each independently —H; -alkyl; -cycloalkyl; -aryl; -alkylenearyl; -alkenylenearyl; -alkynylenearyl; -heterocyclyl; -heteroaryl; -alkyleneheteroaryl; -alkenyleneheteroaryl; -alkynyleneheteroaryl; -alkyleneheterocyclyl; -alkenyleneheterocyclyl; -alkynyleneheterocyclyl; -heterocyclylalkyl; -heterocyclylaryl; -fused aryl; -fused cycloalkylaryl; -fused arylcycloalkyl; -fused cycloalkylheteroaryl; -fused heteroarylcycloalkyl; -fused heteroaryl; -fused cycloalkylheteroaryl; -fused heteroarylcycloalkyl;

wherein R₁₀ is —H; -alkyl; -aryl; -alkylenealkoxy; or -cycloalkyl;

wherein R₁₁ is -alkyl; -aryl; -alkylenearyl; -alkenylaryl; -heteroaryl; -alkyleneheteroaryl; -alkenylheteroaryl; -heterocyclyl; or -cycloalkyl.

wherein R₁₂ is —H; -alkyl; -cycloalkyl; -heterocyclyl; -alkyleneheterocyclyl; -alkenyleneheterocyclyl; -alkynyleneheterocyclyl; -aryl; -alkylenearyl; -alkenylenearyl; -alkynylenearyl; -heteroaryl; -alkyleneheteroaryl; -alkenylheteroaryl; -alkynyleneheteroaryl; or —NR₂₅R₂₆, where R₂₅ and R₂₆ may be taken together to form a ring having the formula —(CH₂)ₒ-M-(CH₂)ₚ— bonded to the nitrogen atom to which R₁₉ and R₂₀ are attached wherein o and p are independently equal to 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—;

wherein R₁₃, and R₁₄ are each independently —H; -alkyl; -aryl; -heterocyclyl; -cycloalkyl; -heteroaryl; -alkylenearyl; -alkenylaryl; -alkyleneheteroaryl; -alkenylheteroaryl; -fused aryl; -fused heteroaryl; -fused cycloalkylaryl; -fused arylcycloalkyl; -fused heterocyclylaryl; -fused arylheterocyclyl; -fused heteroarylcycloalkyl; -fused heterocyclylheteroaryl; or -fused heteroarylheterocyclyl;

wherein B, C and E are each independently —(CH₂)ₙ—, n=0, 1, 2, 3, 4;

wherein F is —(CH₂)ₙ—, n=0, 1, 2, 3, 4;

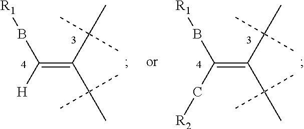

where the 3 & 4 centers may possess R or S or RS configuration when the bonds are saturated;

wherein R₁ and R₂ are each independently —H; -alkyl; -alkoxy; -alkenyl; -alkynyl; -cycloalkyl; -heterocyclyl; -aryl; -aryloxy; -alkenylenearyl; -alkenylenearyl; -alkynylenearyl; -heteroaryl; -alkyleneheteroaryl; -alkenylheteroaryl; -alkynyleneheteroaryl; -fused carbocyclic; -fused aromatic; -fused heteroaromatic; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; or -fused heteroarylheterocyclyl; alternatively, R₁ and R₂ may together form a cycloalkyl or heterocyclic ring.

wherein D is —(CH₂)ₙ—, n=0, 1, 2, 3, 4; —(CH═CH)ₙ—, n=0, 1, 2; —(CH═CR₅)—; —C(O)—; —C(O)—C(O)—; or —S(O)₂—;

wherein R₃ is —H; —C(O)OH; —C(O)OR₁₉; —C(O)NR₂₂R₂₃; —S(O)₂NHC(O)-alkyl; —S(O)₂NHC(O)-aryl; —S(O)₂NHC(O)-heteroaryl; —S(O)₂NHC(O)-alkylenearyl; —S(O)₂NHC(O)-alkyleneheteroryl; —S(O)₂NHC(O)-arylenealkyl; an acid isostere; —CHR₁₃R₁₄; —CN; —P(O)(OH)₂; —P(O)(OH)(O-alkyl); —P(O)(O-alkyl)₂; -alkyl; -cycloalkyl: -heterocyclyl; -aryl; -aryloxy; -cycloalkylaryl; -heteroaryl; -alkyleneheteroaryl; -alkenyleneheteroaryl; -alkynyleneheteroaryl; -alkylenearyl; -alkenylenearyl; -alkynylenearyl; -fused carbocyclic; -fused aromatic; -fused heteroaromatic; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; -fused heteroarylheterocyclyl;

wherein R₄ is -hydrogen: -alkyl; -alkoxy; -alkenyl; -alkynyl; -cycloalkyl; -heterocyclyl; -aryl; -aryloxy; -alkenylenearyl; -alkenylenearyl; -alkynylenearyl; -heteroaryl; -alkyleneheteroaryl; -alkenylheteroaryl; -alkynyleneheteroaryl; -fused carbocyclic; -fused aromatic; -fused heteroaromatic; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; or -fused heteroarylheterocyclyl.

In another embodiment, the monocyclic aryl rings and fused aryl rings of compound I optionally comprise from about 1 to about 3 substituents and from about 1 to about 8 substituents, respectively. The substituents are, each independently, —H; -halo; —NR₂₂R₂₃; —NO₂; —OH; —CN; —COOR₁₉; -carbamoyl; -sulfomoyl; -alkoxy; -perhaloalkoxy; —K-alkyl; —K-cycloalkyl; —K-perhaloalkyl; —K-heterocyclyl; —K-aryl; —K-heteroaryl; —K-alkylene-heteroaryl; —K-alkylene-aryl; —K-arylene-alkyl; —K-alkylene-L-R₂₄; —K-cycloalkylene-L-R₂₄; —K-heterocyclylene-L-R₂₄; —K-arylene-L-R₂₄; —K-heteroarylene-L-R₂₄; —K-alkylene-C(O)NR₂₅R₂₆; —K-alkylene-NR₂₅R₂₆; —K-cycloalkylene-alkyl; —K-alkylene-cycloalkyl; -aryloxy-aryl; -aryloxy-alkyl; -alkoxy-alkyl; -alkoxy-aryl; -alkoxy-heteroaryl; -aryloxy-heteroaryl;

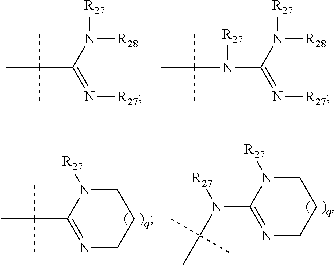

wherein q=0, 1, 2 and 3 and wherein K and L are each independently: -alkylene-; -arylene-; -heteroarylene-; —(CH₂)_{d}—, d=0 or 1; —O—; —N(R₂₇)—; —S—; —S(O)—; —S(O)₂—; —O—S(O)—; and —O—C(O)—; —C(O)—O—; —N(R₂₇)C(O)—; —C(O)N(R₂₇)—; —N(R₂₇)C(O)N(R₂₈)—; —N(R₂₇)S(O)₂—; —S(O)₂N(R₂₇)—; —N(R₂₇)S(O)₂N(R₂₈)—; —C(O)N(R₂₇)S(O)₂—; —N(R₂₇)C(O)—O—; —O—C(O)N(R₂₇)—; —N═N—; —N(R₂₇)—N(R₂₈)—.

In yet another embodiment, the stereochemistry of Compound I may be, independently, R, S or RS for stereocenters 1, when X is a sulfoxide, 2 and 3 and 4, when 3 and 4 are saturated. Alternatively, when stereocenters 3 and 4 are unsaturated, the stereochemistry can be an E, Z or EZ configuration.

In a further embodiment of the present invention, there is provided Compound Ia, comprising the formula:

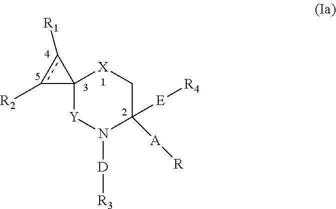

wherein R, R₁, R₂, R₃, R₄, A, D, E, X, and Y are as defined for Compound I.

In yet another embodiment of the present invention, there is provided Compound Ib, comprising the formula:

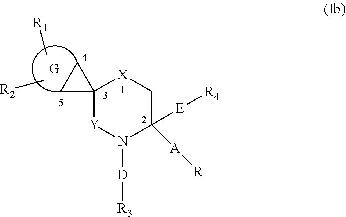

wherein R, R₁, R₂, R₃, R₄, A, D, E, X, and Y groups are as defined for Compound I and G is selected from a group of ring systems consisting of -cycloalkyl; -heterocyclyl; -aryl; or -heteroaryl. The stereocenters 4 and 5 may each, independently, have R or S configurations.

In another embodiment of the present invention, there is provided Compound Ic, comprising the formula:

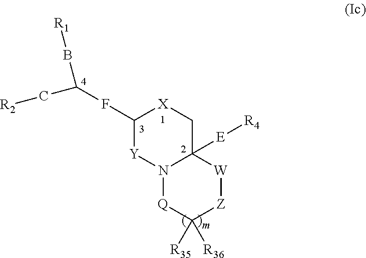

wherein R₁, R₂, R₄, R₃₅, R₃₆, B, C, E, F, X, and Y are as defined for Compound I and wherein W is —C(O)—; —S(O)₂—; or —(CH₂)ₙ—, n=0, 1, 2, or 3; Z is —O—; or —N—; or —S—; —S(O)—, —S(O)₂—; or —(CH₂)ₙ—, n=0, 1, 2, or 3; Q is —C(O)—; —S(O)₂— or —(CH₂)ₙ—, n=0, 1, 2, or 3; and wherein m=1. Alternatively, m=0, 1, 2, or 3 and wherein R₃₅ and R₃₆ are independently —H; -alkyl; -cycloalkyl; -aryl; -alkylenearyl; -alkenylenearyl; -alkynylenearyl; -heterocyclyl; -alkyleneheterocyclyl; -alkenyleneheterocyclyl; -alkynyleneheterocyclyl.

In an alternate embodiment, R₃₅ and R₃₆ may be taken together to form a ring having the formula —(CH₂)ₒ-M-(CH₂)ₚ— bonded to the same atom to which R₃₅ and R₃₆ are attached wherein o and p are independently 0, 1, 2, 3 or 4 and M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—, wherein R₂₇ and R₂₈ are as defined for Compound I.

In yet another embodiment, Q and Z of Compound Ic may be connected by —C(O)—; —C(O)(CH2)ₙ—; —(CH₂)ₙC(O)—; —S(O)₂(CH₂)ₙ—; or —(CH₂)S(O)₂— where n=0, 1, 2 or 3.

In a further embodiment of the present invention, there is provided Compound Id, comprising the formula:

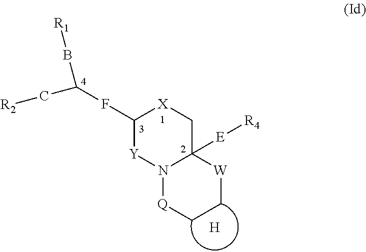

wherein R₁, R₂, R₄, B, C, E, F, X, and Y are as defined for Compound I and W and Q as defined for Compound Ic;

where H is selected from the group of ring systems consisting of -cycloalkyl; -heterocyclyl; -aryl; -heteroaryl.

In another embodiment of the present invention, there is provided Compound Ie, comprising the formula:

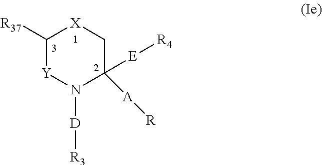

wherein R, R₃, R₄, A, D, E, X, and Y are as defined for Compound I and R₃₇ is —H; -alkyl; -cycloalkyl; -aryl; -alkylenearyl; -alkenylenearyl; -alkynylenearyl; -alkyleneheterocyclyl; -alkenyleneheterocyclyl; or -alkynyleneheterocyclyl.

In a yet another embodiment of the present invention, there is provided Compound If, having the formula:

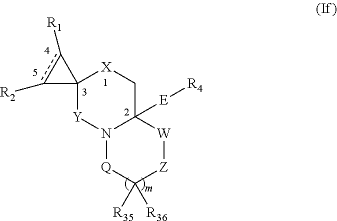

wherein R₁, R₂, R₄, E, X, and Y are as defined for Compound I and all other substituents and modifications are defined as for Compound Ic.

In a further embodiment of the present invention, there is provided Compound Ig, comprising the formula:

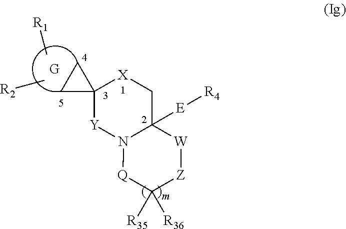

wherein R₁, R₂, R₄, E, X, and Y are defined as for Compound I, W, Z, and Q defined as for Compound Ic and G as defined for Compound Ib. All other substituents and modifications are defined as for Compound Ic.

In an embodiment of the present invention, there is provided Compound Ih, comprising the formula:

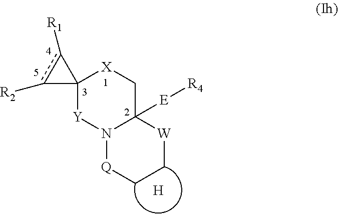

wherein R₁, R₂, R₄, E, X, and Y groups are defined as for Compound I, W and Q are defined as for Compound Ic and H is defined as for Compound Id.

In an embodiment of the compound of Formula (I), the compound of Formula (I) has the Formula (Ii):

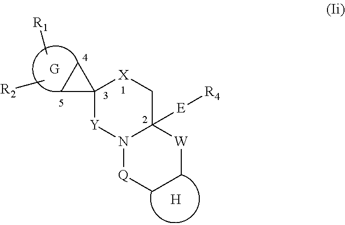

wherein R₁, R₂, R₄, E, X, and Y are defined as for Compound I, G is defined as for Compound Ib, W and Q are defined as for Compound Ic and H is defined as for Compound Id.

In yet another embodiment of the present invention, there is provided Compound Ij, comprising the formula:

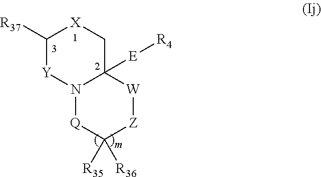

wherein R₄, E, X, and Y groups are defined as for Compound I, W, Z, and Q are defined as for Compound Ic and R₃₇ is defined as for Compound Ie. All other substituents and modifications are defined as for Compound Ic.

In a further embodiment of the present invention, there is provided Compound Ik, comprising the formula:

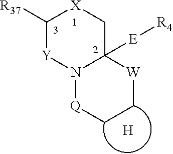

wherein R₄, E, X, and Y groups are as defined for Compound I, W, and Q are defined as Compound Ic and R₃₇ is defined as for Compound Ie.

As used herein, the term ‘alkyl’ refers to a straight chain or branched chain hydrocarbon having from one to twelve carbon atoms. The term ‘alkylene’ refers to a straight or branched chain divalent hydrocarbon radical having from one to twelve carbon atoms. The term ‘alkyline’ refers to a straight or branched chain trivalent hydrocarbon radical having from one to twelve carbon atoms. Alkyl, alkylene and alkyline groups may be optionally substituted with groups chosen from lower alkyl, lower alkoxy, lower alkylsulfanyl, lower alkylsulfenyl, lower alkylsulfonyl, oxo, hydroxy, amino, mercapto optionally substituted with alkyl, carboxy, carbamoyl optionally substituted with alkyl, aminosulfonyl optionally substituted with alkyl, silyloxy optionally substituted with alkyl, alkoxy or aryl, silyl optionally substituted by alkyl, alkoxy or aryl, nitro, cyano, halogen or lower perfluoroalkyl, multiple degrees of substitution being allowed. These alkyl, alkylene, and alkyline groups may contain one or more O, S, S(O) or S(O)₂ atoms. The term ‘lower’ refers to a group containing 1-12 carbon atoms. Non-limiting examples of ‘alkyl’ as used herein include methyl, n-propyl, isopropyl, n-butyl, iso-butyl, tert-butyl, n-pentyl, neo-pentyl and the like. Example of ‘alkylene’ used herein include, but not limited to, methylene, ethylene, propylene, iso butylenes and the like. Non-limiting examples of ‘alkyline’ as used herein include methane, 1,1,2-ethyline, and the like.

As used herein, the term ‘alkenyl’ refers to a hydrocarbon radical having from two to twelve carbons and at least one carbon-carbon double bond. The term ‘alkenylene’ refers to a straight or branched chain divalent hydrocarbon radical having two to twelve carbon atoms and one or more double bonds. The term ‘alkenyline’ refers to a hydrocarbon triradical having from two to twelve carbon atoms and at least one carbon-carbon double bond. The alkenyl, alkenylene and alkenyline groups may be optionally substituted with groups chosen from lower alkyl, lower alkoxy, lower alkylsulfanyl, lower alkylsulfenyl, lower alkylsulfonyl, oxo, hydroxy, amino, mercapto optionally substituted with alkyl, carboxy, carbamoyl optionally substituted with alkyl, aminosulfonyl optionally substituted with alkyl, silyloxy optionally substituted with alkyl, alkoxy or aryl, silyl optionally substituted by alkyl, alkoxy or aryl, nitro, cyano, halogen or lower perfluoroalkyl, multiple degrees of substitution being allowed. Such ‘alkenyl’, ‘alkenylene’, and ‘alkenyline’ groups may contain one or more O, S, S(O) or S(O)₂ atoms. Non-limiting examples of ‘alkenylene’ as used herein include ethene-1,2-diyl, propene-1,3-diyl and the like. Non-limiting examples of ‘alkenyline’ used herein include 1,1,3-propene-1,1,2-triyl, ethene-1,1,2-triyl and the like.

As used herein, the term ‘alkynyl’ refers to a hydrocarbon radical having from two to twelve and at least one triple bond. The term ‘alkynylene’ refers to a straight or branched chain divalent hydrocarbon radical having from two to twelve carbon atoms with one or more carbon-carbon triple bonds. The alkynyl and alkynylene groups may be optionally substituted with groups chosen from lower alkyl, lower alkoxy, lower alkylsulfanyl, lower alkyl sulfenyl, lower sulfonyl, oxo, hydroxy, amino, mercapto optionally substituted with alkyl, carboxy, carbamoyl optionally substituted with alkyl, aminosulfonyl optionally substituted with alkyl, silyloxy optionally substituted with alkyl, alkoxy or aryl, silyl optionally substituted by alkyl, alkoxy or aryl, nitro, cyano, halogen or lower perfluoroalkyl, multiple degrees of substitution being allowed. Such ‘alkynyl’ group may contain one or more O, S, S(O) or S(O)₂ atoms. Non-limiting examples of ‘alkynylene’ as used herein include ethene-1,2-diyl, propyne-1,3-diyl and the like.

As used herein the term ‘cycloalkyl’ refers to an alicyclic hydrocarbon group optionally possessing one or more degrees of unsaturation, having from three to twelve carbon atoms. The term ‘cycloalkylene’ refers to a non-aromatic alicyclic divalent hydrocarbon radical having from three to twelve carbon atoms and optionally possessing one or more degrees of unsaturation. The cycloalkyl and cycloalkylene groups may be optionally substituted with groups chosen from lower alkyl, lower alkoxy, lower alkylsufanyl, lower alkyl sulfenyl, lower sulfonyl, oxo, hydroxy, amino, mercapto optionally substituted with alkyl, carboxy, carbamoyl optionally substituted with alkyl, aminosulfonyl optionally substituted with alkyl, alkoxy or aryl, nitro, cyano, halogen or lower perfluoroalkyl, multiple degrees of substitution being allowed. Non-limiting examples for ‘cycloalkyl’ include cyclopropyl, cyclobutyl, cyclopentyl, cyclohexyl, cycloheptyl or cyclooctyl and the like. Non-limiting examples for ‘cycloalkylene’ include cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl or cyclooctyl-1,5-diyl and the like.

As used herein the term ‘heterocyclyl’ or ‘heterocyclic’ refers to a three to twelve-membered heterocyclic ring. The term ‘heterocyclylene’ refers to a three to twelve membered heterocyclic ring diradical. The heterocyclic or heterocyclyl groups may optionally possess one or more degrees of unsaturation, and must contain one or more heteroatomic substitutions selected from S, S(O), S(O)₂, O, or N, optionally substituted with groups chosen from lower alkyl, lower alkoxy, lower alkylsufanyl, lower alkyl sulfenyl, lower sulfonyl, oxo, hydroxy, amino, mercapto optionally substituted with alkyl, carboxy, carbamoyl optionally substituted with alkyl, aminosulfonyl optionally substituted with alkyl, alkoxy or aryl, nitro, cyano, halogen or lower perfluoroalkyl, multiple degrees of substitution being allowed. Such heterocyclylene or heterocyclic may be optionally fused to one or more of another ‘heterocyclic’ ring(s) or cycloalkyl ring(s) or aryl ring(s). Non-limiting examples of ‘heterocyclic’ include tetrahydrofuran, 1,4-dioxane, piperidine, pyrrolidine, morpholine, piperazine, and like. Non-limiting examples of ‘heterocyclylene’ tetrahydrofuran-2,5-diyl, morpholine-1,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-diyl, and the like.

The term ‘aryl’ as used herein refers to a benzene or an optionally substituted benzene ring system fused to one or more optionally substituted benzene rings. The term ‘arylene’ refers to a benzene ring system diradical fused to one or more optionally substituted benzene rings. The aryl or arylene groups may be optionally substituted with groups chosen from halogen, lower alkyl, lower alkoxy, lower alkylsulfanyl, lower alkylsulfenyl, lower alkylsulfonyl, aryl, oxo, hydroxy, mercapto, amino, optionally substituted with alkyl, carboxy, tetrazoyl, carbamoyl, optionally substituted with alkyl, aminosulfonyl optionally substituted with alkyl, acyl, aroyl, heteroaroyl, acyloxy, aroyloxy, heteraroyloxy, alkoxycarbonyl, silyloxy optionally substituted by alkoxy, alkyl or silyl optionally substituted with alkoxy, alkyl, or aryl, nitro, cyano, halogen, or lower perfluoroalkyl, multiple degrees of substitution is allowed. Non-limiting examples of ‘aryl’ include phenyl, 2-naphthyl, 1-naphthyl, 1-anthracenyl, and the like. Non-limiting examples of ‘arylene’ include benzene-1,4-diyl, naphthalene-1,8-diyl, and the like.

As used herein, the term ‘heteroaryl’ refers to a five to seven membered aromatic ring or to a polycyclic heterocyclic aromatic ring, containing one or more nitrogen, oxygen, or sulfur heteroatoms, where N-oxides, sulfur monoxide and sulfur dioxides are permissible heteroaromatic substitutions. The term ‘heteroarylene’ refers to a five to seven membered aromatic ring diradical, or to a polycyclic heterocyclic aromatic ring diradical, containing one or more nitrogen, oxygen, or sulfur heteroatoms, where N-oxides, sulfur monoxide and sulfur dioxides are permissible heteroaromatic substitutions. The heteroaryl and heteroarylene groups may be optionally substituted with groups chosen from halogen, lower alkyl, lower alkoxy, lower alkylsulfanyl, lower alkylsulfenyl, lower alkylsulfonyl, aryl, oxo, hydroxy, mercapto, amino, optionally substituted with alkyl, carboxy, tetrazoyl, carbamoyl, optionally substituted with alkyl, aminosulfonyl optionally substituted with alkyl, acyl, aroyl, heteroaroyl, acyloxy, aroyloxy, heteraroyloxy, alkoxycarbonyl, silyloxy optionally substituted by alkoxy, alkyl or silyl optionally substituted with alkoxy, alkyl, or aryl, nitro, cyano, halogen, or lower perfluoroalkyl, multiple degrees of substitution is allowed. For polycyclic aromatic ring systems, one or more of the rings may contain one or more heteroatoms. Non-limiting examples of ‘heteroaryl’ include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like. Non-limiting examples of ‘heteroarylene’ may be furan-2,5-diyl, thiophene-2,4-diyl, 1,3,4-oxadiazole-2,5-diyl, 1,3,4-thiadiazole-2,5-diyl, 1,3-thiazole-2,4-diyl, pyridine-2,3-diyl, pyridine-2,5-diyl, pyrimidine-2,5-diyl, pyrimidine-2,4-diyl, quinoline-2,3-diyl, and the like.

The term ‘fused cycloalkylaryl’ as used herein refers to one or more cycloalkyl groups fused to an aryl group, the aryl and cycloalkyl groups having two atoms in common, and wherein the aryl group is the point of substitution. Non-limiting examples of ‘fused cycloalkylaryl’ used herein include:

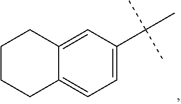

5-indanyl, 5,6,7,8-tetrahydro-2-naphthyl, and like.

The term ‘fused cycloalkylarylene’ as used herein refers to a fused cycloalkylaryl, wherein the aryl group is divalent. Non-limiting examples include

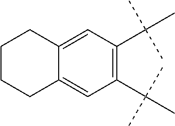

and the like.

The term ‘fused arylcycloalkyl’ as used herein refers to one or more aryl groups fused to a cycloalkyl group, the cycloalkyl and aryl groups having two atoms in common, and wherein the cycloalkyl group is the point of substitution, and wherein the cycloalkyl group is the point of substitution. Non-limiting examples of ‘fused arylcycloalkyl’ used herein include 1-indanyl, 2-indanyl, 9-fluorenyl, 1-(1,2,3,4-tetrahydronaphthyl),

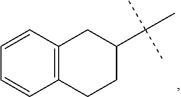

and the like.

The term ‘fused arylcycloalkylene’ as used herein refers to a fused arylcycloalkyl, wherein the cycloalkyl group is divalent. Non-limiting examples include 9,1-fluorenylene,

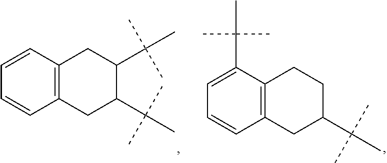

and the like.

The term ‘fused heterocyclylaryl’ as used herein refers to one or more heterocyclyl groups fused to an aryl group, the aryl and heterocyclyl groups having two atoms in common, and wherein the aryl group is the point of substitution. Non-limiting examples of ‘fused heterocyclylaryl’ used herein include 3,4-methylenedioxy-1-phenyl,

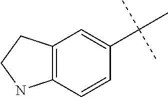

and the like.

The term ‘fused heterocyclylarylene’ refers to a fused heterocyclylaryl, wherein the aryl group is divalent. Non-limiting examples include

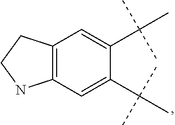

and the like.

The term ‘fused arylheterocyclyl’ as used herein refers to one or more aryl groups fused to a heterocyclyl group, the heterocyclyl and aryl groups having two atoms in common, and wherein the heterocyclyl group is the point of substitution. Non-limiting examples of ‘fused arylheterocyclyl’ used herein include 2-(1,3-benzodioxolyl),

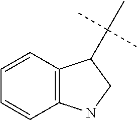

and the like.

The term ‘fused arylheterocyclylene’ refers to a fused arylheterocyclyl wherein the heterocyclyl group is divalent. Non-limiting examples include

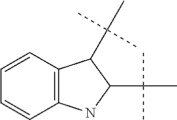

and the like.

The term ‘fused cycloalkylheteroaryl’ as used herein refers to one or more cycloalkyl groups fused to a heteroaryl group, the heteroaryl and cycloalkyl groups having two atoms in common, and wherein the heteroaryl group is the point of substitution. Non-limiting examples of ‘fused cycloalkylheteroaryl’ used herein include 5-aza-6-indanyl,

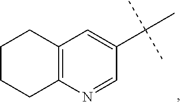

and the like.

The term ‘fused cycloalkylheteroarylene’ as used herein refers to a fused cycloalkylheteroaryl, wherein the heteroaryl group is divalent. Non-limiting examples include

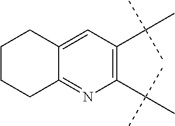

and the like.

The term ‘fused heteroarylcycloalkyl’ as used herein refers to one or more heteroaryl groups fused to a cycloalkyl group, the cycloalkyl and heteroaryl groups having two atoms in common, and wherein the cycloalkyl group is the point of substitution. Non-limiting examples of ‘fused heteroarylcycloalkyl’ include 5-aza-1-indanyl,

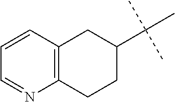

and the like.

The term ‘fused heteroarylcycloalkylene’ as used herein refers to a fused heteroarylcycloalkyl, wherein the cycloalkyl group is divalent. Non-limiting examples include

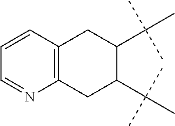

and the like.

The term ‘fused heterocyclylheteroaryl’ as used herein refers to one or more heterocyclyl groups fused to a heteroaryl group, the heteroaryl and heterocyclyl groups having two atoms in common, and wherein the heteroaryl group is the point of substitution. Non-limiting examples of ‘fused heteroarylheterocyclyl include 1,2,3,4,-tetrahydro-beta-carbolin-8-yl,

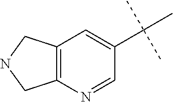

and the like.

The term ‘fused heterocyclylheteroarylene’ as used herein refers to a fused heterocyclylheteroaryl, wherein the heteroaryl group is divalent. Non-limiting examples include

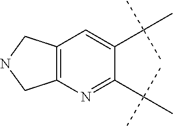

and the like.

The term ‘fused heteroarylheterocyclyl’ as used herein refers to one or more heteroaryl groups fused to a heterocyclyl group, the heterocyclyl and heteroaryl groups having two atoms in common, and wherein the heterocyclyl group is the point of substitution. Examples of ‘fused heteroarylheterocyclyl’ used herein include 5-aza-2,3-dihydrobenzofuran-2-yl,

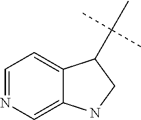

and the like.

The term ‘fused heteroarylheterocyclylene’ as used herein refers to a fused heteroarylheterocyclyl, wherein the heterocyclyl group is divalent. Non-limiting examples include

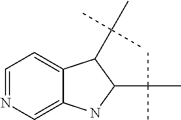

and the like.

The term ‘acid isostere’ as used herein refers to a substituent group, which will ionize at physiological pH to bear a net negative charge. Non-limiting examples of such ‘acid isosteres’ include: 1). Heteroaryl groups such as, but not limited to, isoxazol-3-ol-5-yl, 1H-tetrazole-5-yl, or 2H-tetrazole-5-yl; 2). Heterocyclyl groups such as, but not limited to, imidazoline-2,4-dione-5-yl, imidazolidine-2,4-dione-1-yl, 1,3-thiazolidine-2,4-dione-5-yl, 5-hydroxy-4H-pyran-4-on-2-yl, 1,2,5-thiadiazolidin-3-one-1,1-dioxide-4-yl, 1,2,5-thiadiazolidin-3-one-, -dioxide-5-yl, 1,2,5-thiadiazolidin-3-one-1,1-dioxide-5-yl having substituents at the 2 and/or 4 position; and —N-acyl-alkylsulfonamides.

As used herein, the term ‘alkoxy’ refers to the group RxO—, where Rx is alkyl, the term ‘alkenyloxy’ refers to the group RxO-, where Rx is alkenyl, the term ‘alkynyloxy’ refers to the group RxO—, where Rx is alkynyl, the term ‘alkylsulfanyl’ refers to the group RxS—, where Rx is alkyl and the term ‘alkenylsulfanyl’ refers to the group RxS—, where Rx is alkenyl. Also, the term ‘alkynylsulfanyl’ refers to the group RxS—, where Rx is alkynyl, the term ‘alkylsulfenyl’ refers to the group RxS(O)—, where Rx is alkyl, the term ‘alkenylsulfenyl’ refers to the group RxS(O)—, where Rx is alkenyl, the term ‘alkynylsulfenyl’ refers to the group RxS(O)—, where Rx is alkynyl, and the term ‘alkylsulfonyl’ refers to the group RxS(O)₂—, where Rx is alkyl. The term ‘alkenylsulfonyl’ refers to the group RxS(O)₂—, where Rx is alkenyl the term ‘alkynylsulfonyl’ refers to the group RxS(O)₂—, where Rx is alkynyl, the term ‘acyl’ refers to the group RxC(O)—, where Rx is alkyl, alkenyl, alkynyl, cycloalkyl, cycloalkenyl, or heterocyclyl, and the term ‘aroyl’ refers to the group RxC(O)—, where Rx is aryl. Also as used herein, the term ‘heteroaroyl’ refers to the group RxC(O)—, where Rx is heteroaryl, the term ‘alkoxycarbonyl’ refers to the group RxOC(O)—, where Rx is alkyl, the term ‘acyloxy’ refers to the group RxC(O)O—, where Rx is alkyl, alkenyl, alkynyl, cycloalkyl, cycloalkenyl, or heterocyclyl, the term ‘aroyloxy’ refers to the group RxC(O)O—, where Rx is aryl, and the term ‘heteroaroyloxy’ refers to the group RxC(O)O—, where Rx is heteroaryl.

The terms ‘contain’ or ‘containing’ as used herein may refer to in-line substitutions at any position along the above defined alkyl, alkenyl, alkynyl, or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the term ‘alkyl’ or ‘aryl’ or either of their prefix roots appear in a substituent (example. Arylalkoxyaroyloxy) they shall be interpreted as including those limitations given above for ‘alkyl’ and ‘aryl’. Designated numbers of carbon atoms in an alkyl, alkenyl, or alkynyl or cyclic alkyl moiety or the alkyl portion of larger substituents in which the term ‘alkyl’ appears as its prefix root. The term ‘oxo’ refers to the substituent ═O and the term ‘halo’ or ‘halogen’ include iodine, bromine, chlorine and fluorine. The term ‘mercapto’ refers to the substituent —SH, the term ‘carboxy’ refers to the substituent —COOH, the term ‘cyano’ refers to the substituent —CN, the term ‘aminosulfonyl’ refers to the substituent —SO₂NH₂, and the term ‘carbamoyl’ refers to the substituent —CONH₂. The term ‘sulfanyl’ refers to the substituent —S—, the term ‘sulfenyl’ refers to the substituent —S(O)—, the term ‘sulfonyl’ refers to the substituent —S(O)₂ and the term ‘sulfamoyl’ refers to the substituent

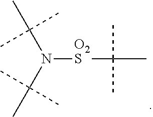

In one embodiment, non-limiting examples of the compounds of the present invention are provided in Table 1.

TABLE 1

| | | | |
|---|---|---|---|
| Compound | R | R₁ | R₂ |

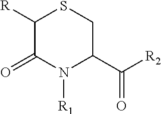

| | | | |
|---|---|---|---|
| 1 | H | H | —OH |
| 2 | H | H | —OMe |
| 3 | H | H | 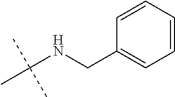 |
| 4 | H | 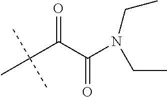 | 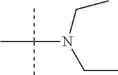 |
| 5 | H | 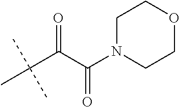 | 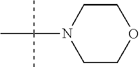 |
| 6 | H | 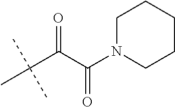 | 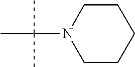 |
| 7 | H | 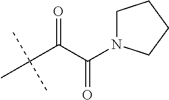 | 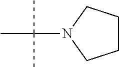 |
| 8 | H | 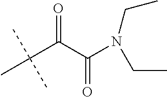 | —OMe |
| 9 | 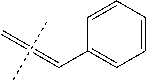 | 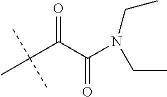 | 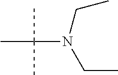 |
| 10 | 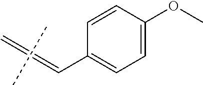 | 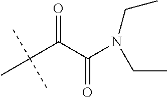 | 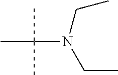 |
| 11 | 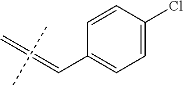 | 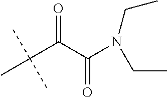 | 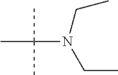 |
| 12 | 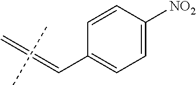 | 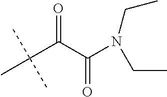 | 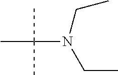 |
| 13 | 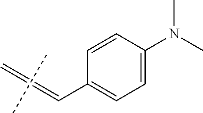 | 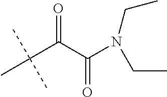 | 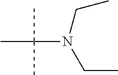 |
| 14 | 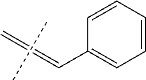 | 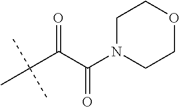 | 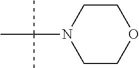 |
| 15 | H | 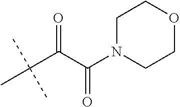 | —OMe |
| 16 | 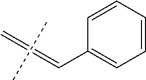 | 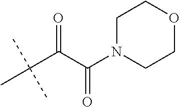 | —OMe |
| 17 | 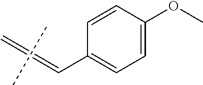 | 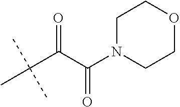 | —OMe |
| 18 | 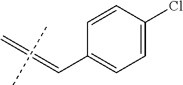 | 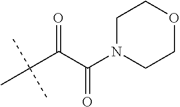 | —OMe |
| 19 | 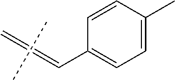 | 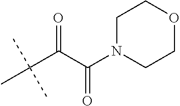 | —OMe |
| 20 | 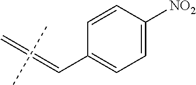 | 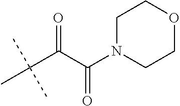 | —OMe |
| 21 | 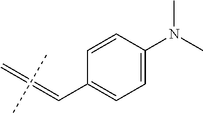 | 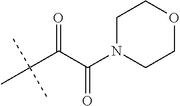 | —OMe |
| 22 | 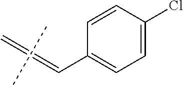 | 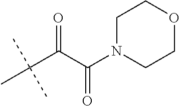 | 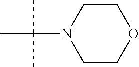 |
| 23 | 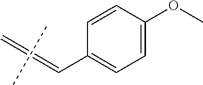 | 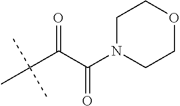 | 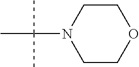 |
| 24 | 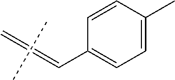 | 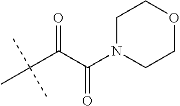 | 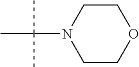 |
| 25 | 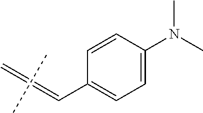 | 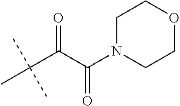 | 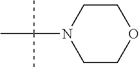 |
| 26 | 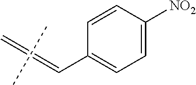 | 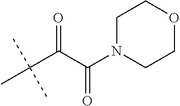 | 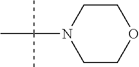 |
| 27 | 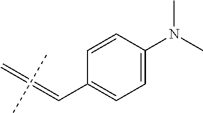 | 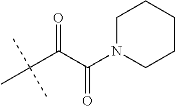 | 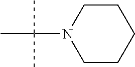 |
| 28 | 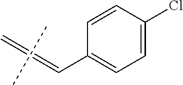 | 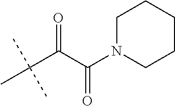 | 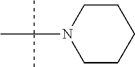 |
| 29 | 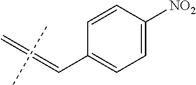 | 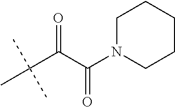 | 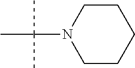 |
| 30 | 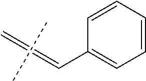 | 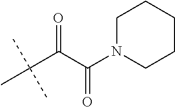 | 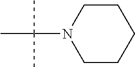 |
| 31 | 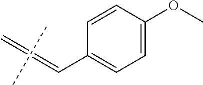 | 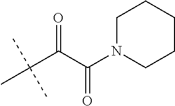 | 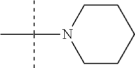 |
| 32 | 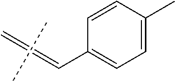 | 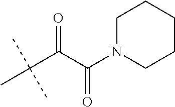 | 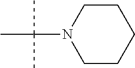 |
| 33 | —H | —H | 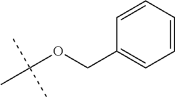 |
| 34 | —H | 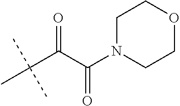 | 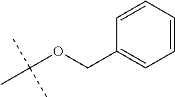 |
| 35 | 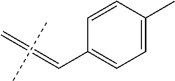 | 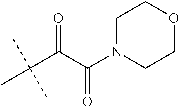 | 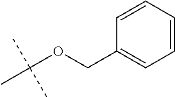 |
| 36 | 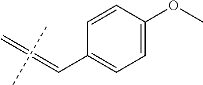 | 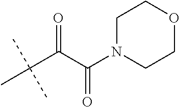 | 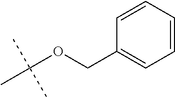 |
| 37 | 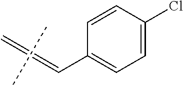 | 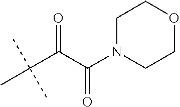 | 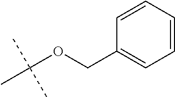 |
| 38 | 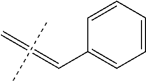 | 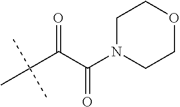 | 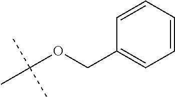 |
| 39 | 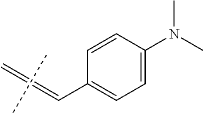 | 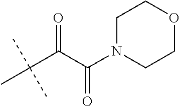 | 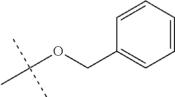 |
| 40 | 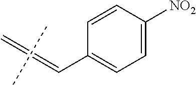 | 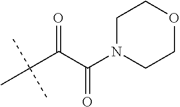 | 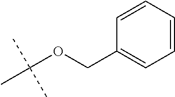 |
| 41 | 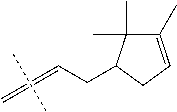 | 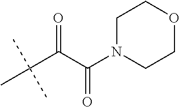 | 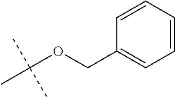 |
| 42 | —H | 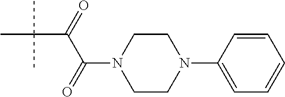 | 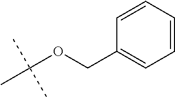 |
| 43 | 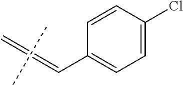 | 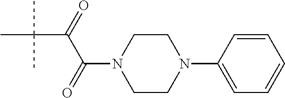 | 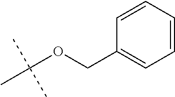 |
| 44 | 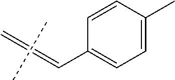 | 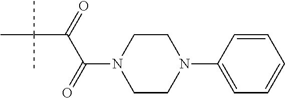 | 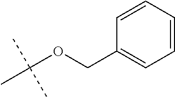 |
| 45 | 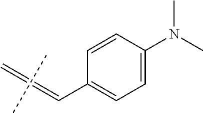 | 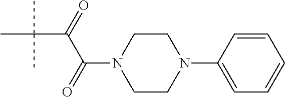 | 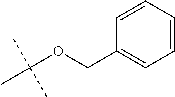 |
| 46 | 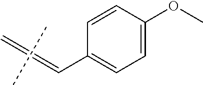 | 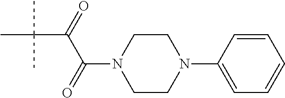 | 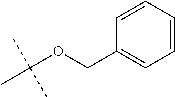 |
| 47 | 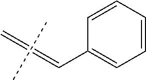 | 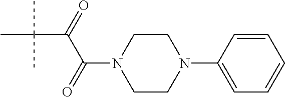 | 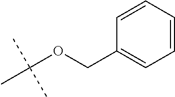 |
| 48 | 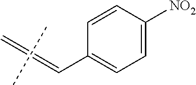 | 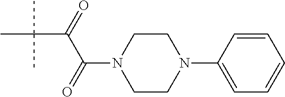 | 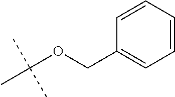 |
| 49 | —H | —H | 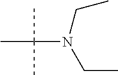 |
| 50 | —H | —H | 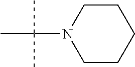 |
| 51 | 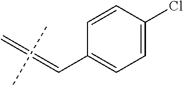 | 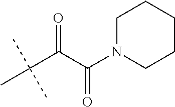 | —OMe |
| 52 | 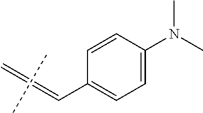 | 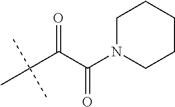 | —OMe |
| 53 | 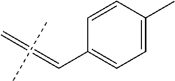 | 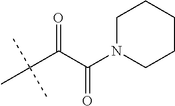 | —OMe |
| 54 | 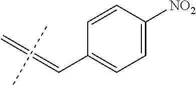 | 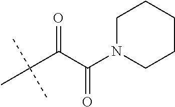 | —OMe |
| 55 | 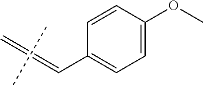 | 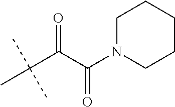 | —OMe |
| 56 | 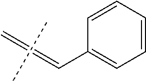 | 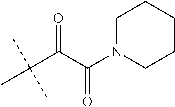 | —OMe |
| 57 | H | 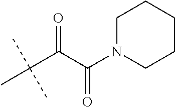 | —OMe |
| 58 | H | 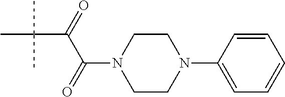 | 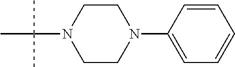 |
| 50 | 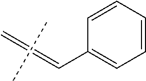 | 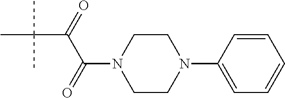 | 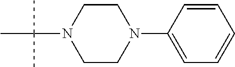 |
| 60 | 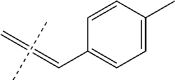 | 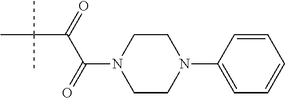 | 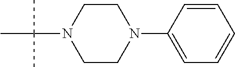 |
| 61 | 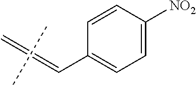 | 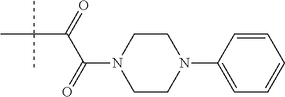 | 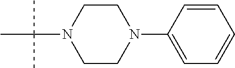 |
| 62 | 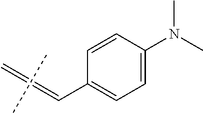 | 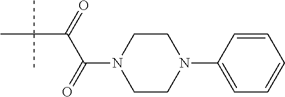 | 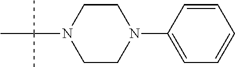 |
| 63 | 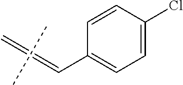 | 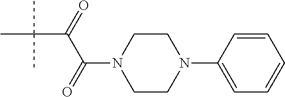 | 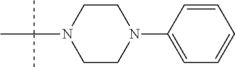 |
| 64 | 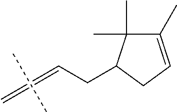 | 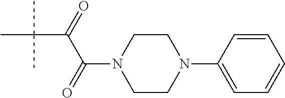 | 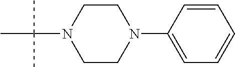 |
| 65 | H | 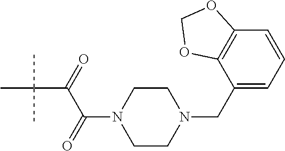 | 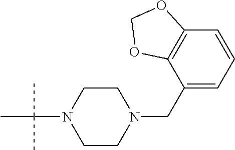 |
| 66 | 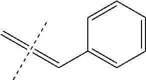 | 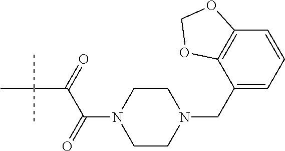 | 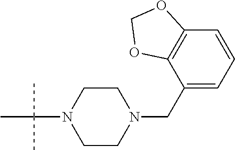 |
| 67 | 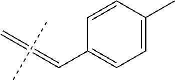 | 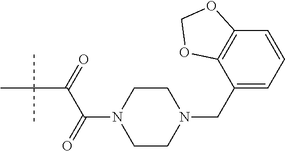 | 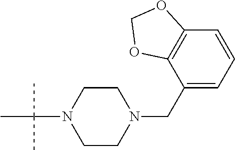 |
| 68 | 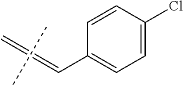 | 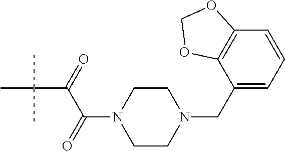 | 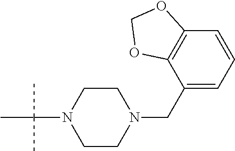 |
| 69 | 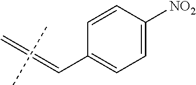 | 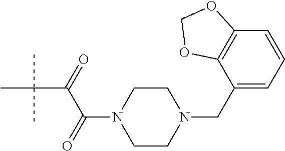 | 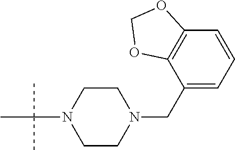 |
| 70 | 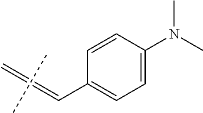 | 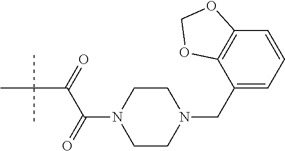 | 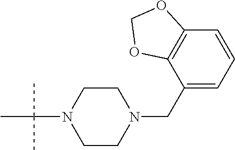 |
| 71 | 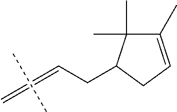 | 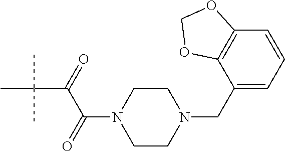 | 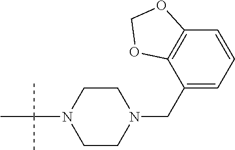 |
| 72 | H | 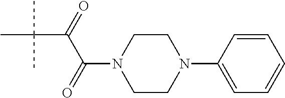 | —OMe |
| 73 | 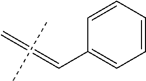 | 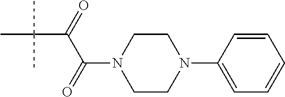 | —OMe |
| 74 | 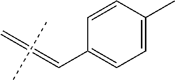 | 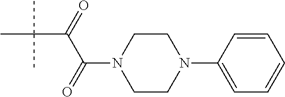 | —OMe |
| 75 | 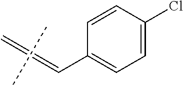 | 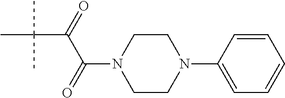 | —OMe |
| 76 | 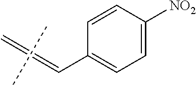 | 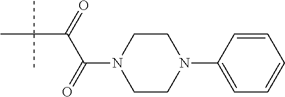 | —OMe |
| 77 | 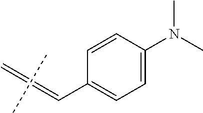 | 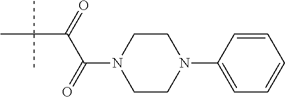 | —OMe |
| 78 | 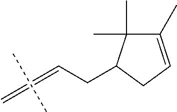 | 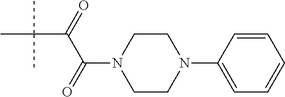 | —OMe |
| 79 | 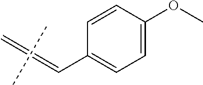 | 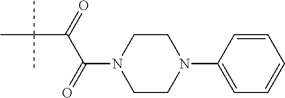 | —OMe |
| 80 | —H | 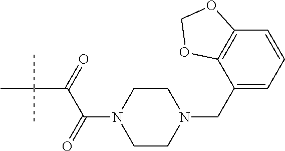 | —OMe |
| 81 | 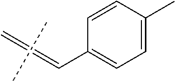 | 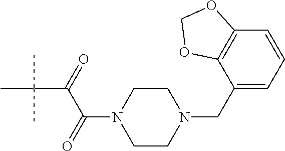 | —OMe |
| 82 | 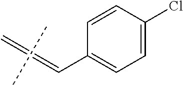 | 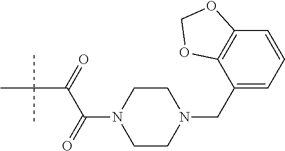 | —OMe |
| 83 | 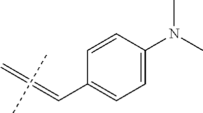 | 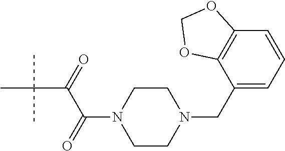 | —OMe |
| 84 | 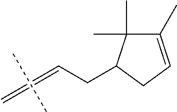 | 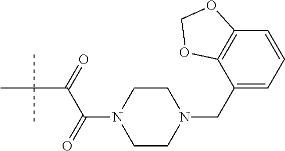 | —OMe |
| 85 | 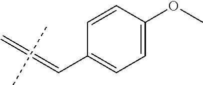 | 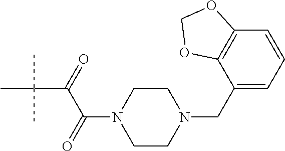 | —OMe |
| 86 | —H | —H | 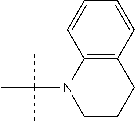 |
| 87 | —H | 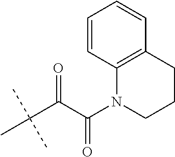 | 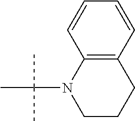 |
| 88 | 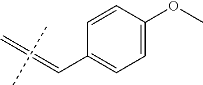 | 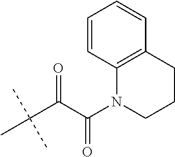 | 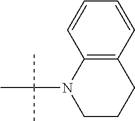 |
| 89 | 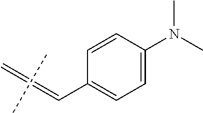 | 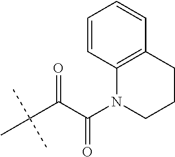 | 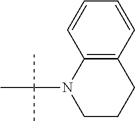 |
| 90 | 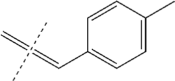 | 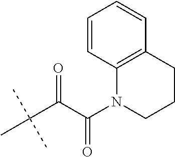 | 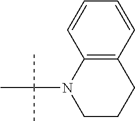 |
| 91 | 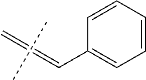 | 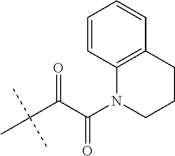 | 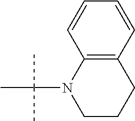 |
| 92 | 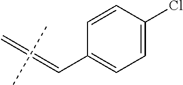 | 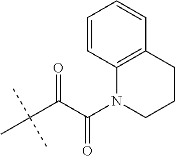 | 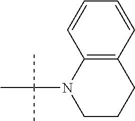 |
| 93 | 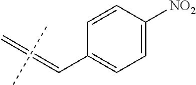 | 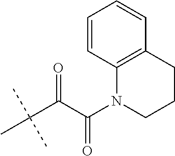 | 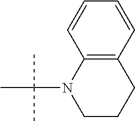 |
| 94 | 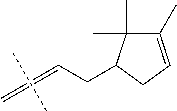 | 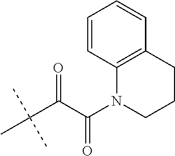 | 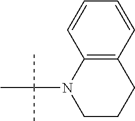 |
| 95 | —H | —H | 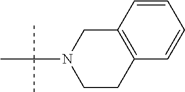 |

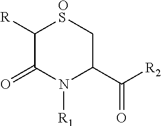

| | | | |
|---|---|---|---|
| 96 | H (isomer 1) | H | —OMe |
| 97 | H (isomer 2) | H | —OMe |
| 98 | H (mixture of isomers) | H | 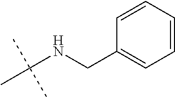 |
| 99 | H (mixture of isomers) | 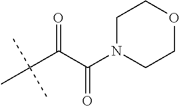 | 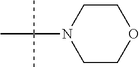 |
| 100 | H (mixture of isomers) | 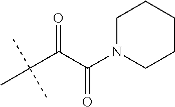 | 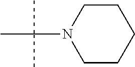 |
| 101 | 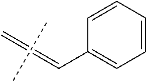 | 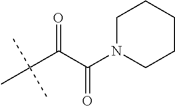 | 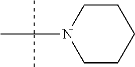 |
| 102 | —H | 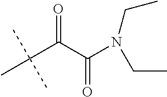 | 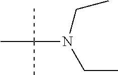 |
| 103 | —H | —H | 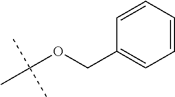 |
| 104 | 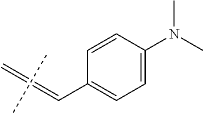 | 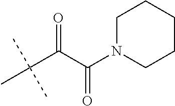 | 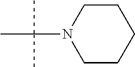 |

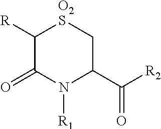

| | | | |
|---|---|---|---|
| 105 | H | H | —OMe |
| 106 | 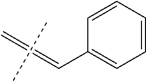 | H | —OMe |
| 107 | 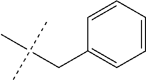 | H | —OMe |
| 108 | 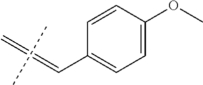 | H | —OMe |
| 109 | 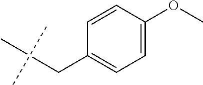 | H | —OMe |
| 110 | 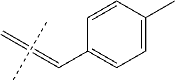 | H | —OMe |
| 111 | 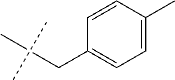 | H | —OMe |
| 112 | 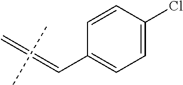 | H | —OMe |
| 113 | 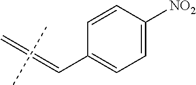 | H | —OMe |
| 114 | 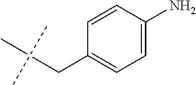 | H | —OMe |
| 115 | H | H | 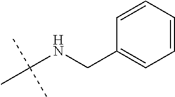 |
| 116 | 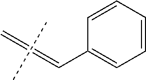 | H | 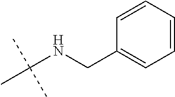 |
| 117 | 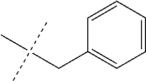 | H | 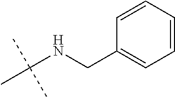 |
| 118 | 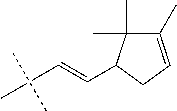 | H | —OMe |
| 119 | 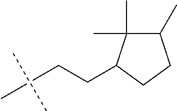 | H | —OMe |
| 120 | 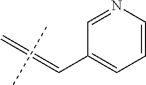 | H | —OMe |
| 121 | —H | —H | 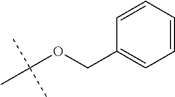 |
| 122 | 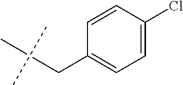 | —H | —OMe |
| 123 | 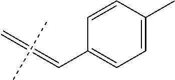 | —H | 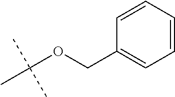 |
| 124 | 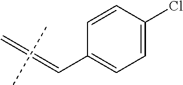 | —H | 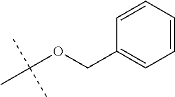 |
| 125 | 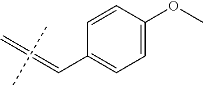 | —H | 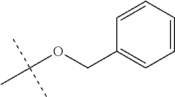 |
| 126 | 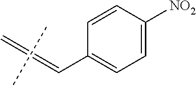 | —H | 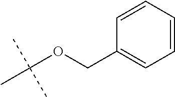 |
| 127 | 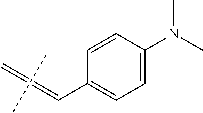 | —H | 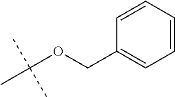 |
| 128 | 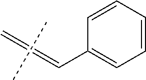 | —H | 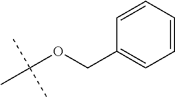 |
| 129 | 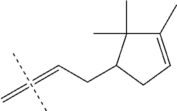 | —H | 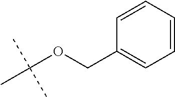 |
| 130 | 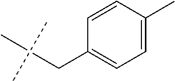 | —H | —OH |
| 131 | 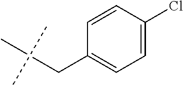 | —H | —OH |
| 132 | 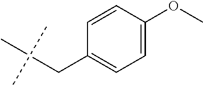 | —H | —OH |
| 133 | 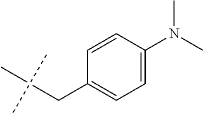 | —H | —OH |
| 134 | 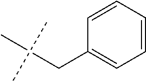 | —H | —OH |
| 135 | 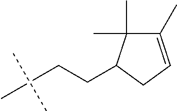 | —H | —OH |
| 136 | —H | 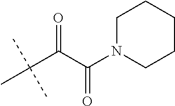 | 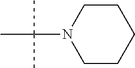 |
| 137 | —H | 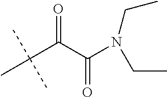 | 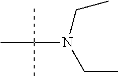 |

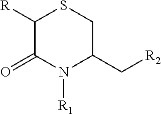

| | | | |
|---|---|---|---|
| 138 | H | H | —OH |
| 139 | H | H | 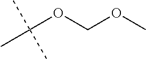 |

In one embodiment of the present invention there is provided a pharmaceutical composition comprising the compounds of the present invention and one or more pharmaceutically acceptable carriers, excipients, or diluents. In an exemplary embodiment, the present invention provides a pharmaceutical composition comprising Compound I and one or more pharmaceutically acceptable carriers, excipients, or diluents. The term “pharmaceutical composition” is used herein to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques. The term “therapeutically effective amount” is used herein to denote that amount of a drug or pharmaceutical agent that will elicit the therapeutic response of an animal or human that is being sought. The skilled artisan will be able to determine the therapeutically effective amount based on a patient's illness and response to the composition.

The pharmaceutical compositions comprising a compound of the present invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically -acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, preservative and flavoring and coloring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient, which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth-washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention, which are generally prepared by reacting the free base with a suitable organic or inorganic acid or by reacting the acid with a suitable organic or inorganic base. Representative salts include the following salts: Acetate, Adipate, Alginate, Aspartate, Benzenesulfonate, Benzoate, Bicarbonate, Bisulfate, Bitartrate, Borate, Bromide, Butyrate, Calcium Camphorate, Camphorsulfonate, Camsylate, Carbonate, Chloride, Clavulanate, Citrate, Cyclopentanepropionate, Dodecylsulfate, Digluconate, Dihydrochloride, Edetate, Edisylate, Estolate, Esylate, Ethanesulfonate, glucoheptanoate, Fumarate, Gluceptate, Gluconate, Glutamate, Glycerophosphate, Glycollylarsanilate, Hemisulfate, Heptanoate, Hexanoate, Hexylresorcinate, Hydrabamine, Hydrobromide, Hydrochloride, Hydroiodide, 2-Hydroxyethanesulfonate, Hydroxynaphthoate, Iodide, Isethionate, Lactate, Lactobionate, Laurate, Malate, Maleate, Mandelate, Methanesulfonate, Methylbromide, Methylnitrate, Mesylate, Methylsulfate, Monopotassium Maleate, Mucate, 2-Naphthalenesulfonate, Napsylate, Nicotinate, Nitrate, N-methylglucamine, Oxalate, Pamoate (Embonate), Palmitate, Pantothenate, Pectinate, Persulphate, 3-Phenylpropionate, Phosphate/diphosphate, Picrate, Pivalate, Propionate, Polygalacturonate, Potassium, Salicylate, Sodium, Stearate, Succinate, Subacetate, Succinate, Tannate, Tartrate, Teoclate, Thiocyanate, Tosylate, Triethiodide, Trimethylammonium, Undecanoate and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (1977) p. 1-19. Also, the basic nitrogen-containing groups can be quaternized with such agents, as lower alkyl halides, such as methyl, ethyl, propyl, butyl chloride, bromides, and iodides; dialkyl sulfates like dimethyl, diethyl, dibutyl and diamyl sulfates, long chain halides such as decyl, lauryl, myristyl, and stearyl chlorides, bromides and iodides, arylalkylhalides like benzyl and phenethyl bromides, and other. Water and oil-soluble or dispersible products are thereby obtained. In an exemplary embodiment, the present invention provides a pharmaceutical formulation comprising a hydrochloric acid salt of Compound I. In an alternate exemplary embodiment, the present invention provides a pharmaceutical formulation comprising a sodium salt of Compound I.

Other salts, which are not pharmaceutically acceptable may be useful in the preparation of compounds of the invention and these form a further aspect of the invention.

In addition, some of the compounds of the present invention may form solvates with water or common organic solvents. Such solvates are also encompassed within the scope of the invention.

In another embodiment, the compounds of the present invention may be prodrugs. The term ‘prodrug’ includes biohydrolyzable amides and biohydrolyzable esters and also encompasses a) compounds in which biohydrolyzable functionality in such a prodrug is encompassed in the compounds of the present invention. For example, the lactam formed by a carboxylic group in R in Compound I and b) compounds which may be oxidized or reduced biologically at a given functional group to yield drug substances of Compound I. Examples of these functional groups include, but are not limited to, 1,4 dihydropyridine, N-alkylcarbonyl-1,4-dihydropyridine, 1,4-cyclohexadiene, tert butyl, and the like.

In yet another embodiment, the present invention provides a pharmaceutical composition comprising a therapeutically effective amount of the compounds of the present invention and one or more pharmaceutically acceptable carriers, excipients, or diluents, further comprising one or more therapeutic agents.

The term “treatment” or “treating” as used herein, refers to the full spectrum of treatments for a given disorder from which the patient is suffering, including alleviation of one, most of all symptoms resulting from that disorder, to an outright cure for the particular disorder or prevention of the onset of the disorder.

The compounds of the present invention may be administered at a dosage level of from about 0.01 to 1000 mg/kg of the body weight of the subject being treated, with a preferred dosage range between 0.01 and 100 mg/kg, most preferably 0.5 to 10 mg/kg of body weight per day. The amount of active ingredient that may be combined with the carrier materials to produce a single dosage will vary depending upon the host treated and the particular mode of administration. For example, a formulation intended for oral administration to humans may contain 1 mg to 2 grams of Compound I with an appropriate and convenient amount of carrier material that may vary from about 5 to 95 percent of the total composition. Dosage unit forms will generally contain between from about 5 mg to about 500 mg of active ingredient. This dosage has to be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

While the invention has been described and illustrated with reference to certain preferred embodiments thereof, those skilled in the art will appreciate that various changes, modifications and substitutions can be made therein without departing from the spirit and scope of the invention. For example, effective dosages other than the preferred dosages as set forth herein may be applicable as a consequence of variations in the responsiveness of the mammal being treated for metabolic disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

The embodiments of the present invention may be better understood by the following non-limiting examples.

### EXAMPLES

The following examples describe the synthesis of the compounds of Table 1.

### Preparation of 5-oxa-thiomorpholine-3-carboxylic acid (Compound 1)

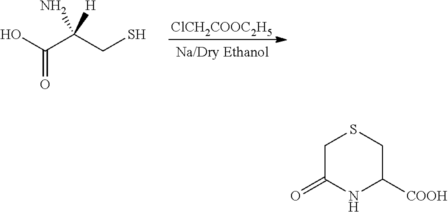

To a suspension of L-cysteine (10 mmol) in dry ethanol (40 ml) under nitrogen were added sodium chips (21 mmol) portion wise during 15 min. Once all the solids are dissolved, the chloroester (10 mmol) was added drop wise. The reaction mixture (RM) was stirred for 10 h. The RM was acidified with 3N HCl (P^{H}=3-4). The crude mixture was partially evaporated and extracted with ethyl acetate. The organic layers were collected, dried (Na₂SO₄) and concentrated to obtain white solid.

Compound 1: HRMS (m/z): 184 (M+Na); ¹H NMR (300 MHz, CD₃OD): δ 3.06-3.52 (m, 5H), 4.44-4.47 (t, 1H); ¹³CNMR (75 MHz, CD₃OD): δ 28.11, 30.05, 57.57, 169.53, 173.01.

### Preparation of Methyl-5-oxa-thiomorpholine-3-carboxylate (Compound 2)

5-oxa-thiomorpholine-3-carboxylic acid 1 (5 mmol) was taken in a round bottomed flask and 15 ml of dry ether was added to it. Diazomethane (40 mmol) was added at 0° C. and stirred for 5-6 h. After completion of the reaction ether was evaporated from the reaction mixture to obtain Compound 2.

### General Procedure for Synthesis of Compounds 3-7, 58 & 65

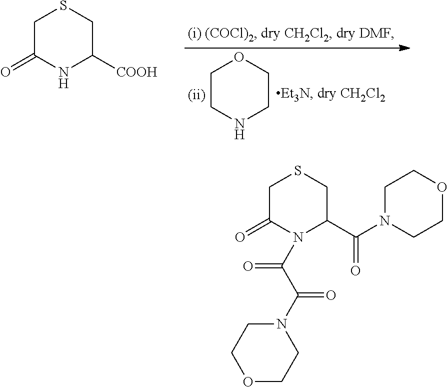

To a suspension of 5-oxa-thiomorpholine-3-carboxylic acid 1 (1 mmol) in 5 ml of dry dichloromethane with catalytic amount of dry dimethylformamide (0.02 mmol), was added oxalyl chloride (3 mmol). The RM was stirred at room temperature (RT) for 3 h and concentrated, dried under vacuum. The RM was charged with dry dichloromethane (5 ml) and kept at 0° C. Amine (2 nmol) and triethyl amine (2 mmol) were added to the RM and stirred at RT for 2 h. The RM was diluted with additional dichloromethane and washed with small volumes of 3N HCl and then washed with saturated NaHCO₃ followed by brine. Organic layer was separated and dried over anhydrous Na₂SO₄ and concentrated to get the crude and it was purified on a silica gel column to obtain the amides. Compound 3 was obtained from the same procedure using benzyl amine.

Compound 3: HRMS (m/z): 273 (M+Na); ¹H NMR (300 MHz, CDCl₃): δ 2.86-3.17 (m, 4H), 4.18-4.19 (m, 1H), 4.33-4.49 (m, 2H), 7.22-7.28 (m, 5H), 7.39 (br s, 1H), 7.48 (br s, 1H); ¹³CNMR (75 MHz, CDCl₃): δ 28.08, 30.10, 43.87, 58.30, 127.62, 127.86, 128.69, 137.88, 167.85, 169.60.

Compound 4: HRMS (m/z): 366 (M+Na); ¹H NMR (400 MHz, CDCl₃): δ 1.09-1.20 (m, 9H), 1.31-1.35 (t, 3H), 2.88-2.89 (dd, 1H), 3.12-3.56 (m, 11H), 5.34-5.37 (t, 1H); ¹³CNMR (100 MHz, CDCl₃): δ 11.68, 12.88, 13.02, 14.26, 28.13, 32.35, 38.28, 41.13, 41.79, 42.23, 55.24, 163.30, 167.53, 168.19.

Compound 5: HRMS (m/z): 394 (M+Na); ¹H NMR (400 MHz, CDCl₃): δ 2.82-2.87 (dd, 1H), 3.27-3.73 (m, 20H), 5.40-5.43 (t, 1H); ¹³CNMR (75 MHz, CDCl₃): δ 27.37, 32.01, 41.24, 42.95, 45.73, 46.09, 54.97, 65.67, 66.29, 162.39, 166.85, 166.90, 168.21.

Compound 6: HRMS (m/z): 390 (M+Na); ¹H NMR (400 MHz, CDCl₃): δ 1.53-1.71 (m, 12H), 2.83-2.88 (dd, 1H), 3.21-3.61 (m, 11H), 5.49-5.51 (t, 1H); ¹³CNMR (100 MHz, CDCl₃): δ 24.38, 24.46, 24.70, 24.96, 25.47, 26.36, 27.63, 31.96, 41.94, 44.14, 46.82, 46.94, 54.81, 162.53, 166.45, 167.48, 168.35.

Compound 7: HRMS (m/z): 362 (M+Na); ¹H NMR (400 MHz, CDCl₃): δ 1.89-2.09 (m, 8H), 2.88-3.82 (m, 12H), 5.24-5.30 (m, 1H).

Compound 58: HRMS (m/z): 544 (M+Na); ¹H NMR (400 MHz, CDCl₃): δ 2.89 (m, 1H), 3.22-3.90 (m, 19H), 5.57 (t, 1H), 6.89-6.97 (m, 6H), 7.26-7.33 (m, 4H); ¹³CNMR (100 MHz, CDCl₃): δ 27.77, 32.22, 41.14, 42.94, 45.60, 45.90, 48.71, 48.80, 49.52, 49.71, 55.28, 116.85, 116.87, 120.54, 120.95, 129.25, 129.35, 150.66, 150.96, 162.55, 166.90, 167.28, 168.21.

Compound 65: HRMS (m/z): 660 (M+Na); ¹H NMR (400 MHz, CDCl₃): δ 2.28-2.51 (m, 8H), 3.26-3.75 (m, 16H), 5.50 (t, 1H), 5.94 (d, 4H), 6.71-6.84 (m, 6H); ¹³CNMR (100 MHz, CDCl₃): δ 27.62, 32.09, 41.13, 42.92, 45.71, 45.88, 51.65, 51.76, 52.43, 52.78, 55.00, 62.41, 62.50, 100.93, 100.99, 107.91, 107.97, 109.32, 109.42, 122.24, 122.28, 131.22, 131.50, 146.75, 146.87, 147.70, 147.78, 162.45, 166.61, 167.25, 168.19.

### General Procedure for Synthesis of Compounds 9-14, 22-32, 59-64, 6-71, and 88-94

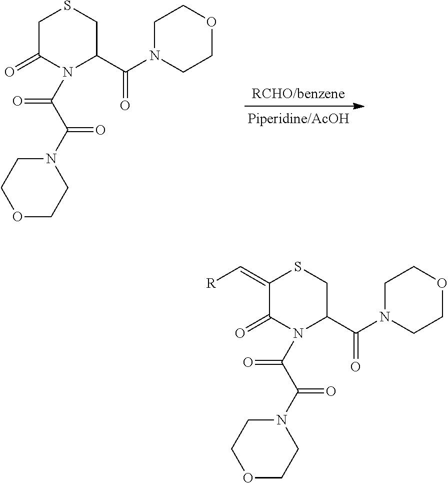

To a solution of diamide (1 mmol) in 5 ml of dry benzene were added piperidine and acetic acid (80 mmol each) followed by aldehyde (1.5 mmol). The RM was refluxed at 100° C. using a Dean-Stark set up for 1 h. After completion of the reaction, benzene was completely removed from the RM and ethyl acetate was added to the crude. The mixture was washed with small volumes of 3N HCl and with saturated NaHCO₃ solution followed by brine. Organic layer was dried over anhydrous Na₂SO₄ and then concentrated to get the crude product, which was purified on a silica gel column.

Compound 9: HRMS (m/z): 454 (M+Na); ¹H NMR (400 MHz, CDCl₃+CCl₄): δ 1.07-1.32 (m, 12H), 3.17-3.18 (dd, 1H), 3.24-3.53 (m, 9H), 3.78 (s, 3H), 5.50-5.52 (t, 1H), 7.25-7.34 (m, 3H), 7.52-7.54 (d, 2H), 8.03 (s, 1H); ¹³CNMR (100 MHz, CDCl₃+CCl₄): δ 12.00, 12.94, 13.12, 14.36, 27.57, 38.28, 41.06, 41.94, 42.19, 54.36, 122.85, 128.29, 129.19, 130.74, 134.66, 137.80, 163.12, 163.64, 166.37, 167.67.

Compound 10: HRMS (m/z): 484 (M+Na); ¹H NMR (400 MHz, CDCl₃): δ 1.12-1.38 (m, 12H), 3.07-3.55 (m, 11H), 3.83 (s, 3H), 5.56-5.58 (t, 1H), 6.91-6.93 (d, 2H), 7.59-7.61 (d, 2H), 8.08 (s, 1H); ¹³CNMR (100 MHz, CDCl₃): δ 12.02, 12.87, 13.07, 14.29, 27.67, 38.31, 41.14, 42.00, 42.29, 54.66, 55.34, 113.87, 119.90, 127.4, 132.87, 138.17, 160.46, 163.61, 163.89, 166.55, 167.85.

Compound 11: HRMS (m/z): 488 (M+Na); ¹H NMR (300 MHz, CDCl₃): δ 1.11-1.42 (m, 12H), 3.03-3.59 (m, 10H), 5.61-5.64 (t, 1H), 7.34-7.37 (d, 2H), 7.52-7.55 (d, 2H), 8.02 (s, 1H); ¹³CNMR (75 MHz, CDCl₃): δ 11.92, 12.86, 13.03, 14.28, 27.54, 38.38, 41.14, 42.03, 42.27, 54.09, 123.49, 128.60, 131.86, 133.14, 135.08, 136.10, 163.07, 163.69, 166.31, 167.69.

Compound 12: HRMS (m/z): 499 (M+Na); ¹H NMR (400 MHz, CDCl₃): δ 1.11-1.38 (m, 12H), 3.13-3.59 (m, 10H), 5.69-5.71 (t, 1H), 7.72-7.74 (d, 2H), 8.21-8.23 (d, 2H), 8.04 (s, 1H); ¹³CNMR (100 MHz, CDCl₃): δ 11.93, 12.92, 13.09, 14.37, 27.44, 38.45, 41.20, 42.10, 42.35, 53.42, 123.59, 127.31, 131.06, 133.58, 140.95, 147.10, 162.51, 163.52, 165.96, 167.46.

Compound 13: HRMS (m/z): 497 (M+Na); ¹H NMR (400 MHz, CDCl₃): δ 1.11-1.35 (m, 12H), 3.02 (s, 6H), 3.05-3.52 (m, 10H), 5.49 (t, 1H), 6.66-6.68 (d, 2H), 7.57-7.59 (d, 2H), 8.07 (s, 1H); ¹³CNMR (100 MHz, CDCl₃): δ 12.10, 12.88, 13.08, 14.30, 28.00, 38.28, 40.04, 41.16, 42.00, 42.32, 55.33, 111.29, 116.10, 122.61, 133.31, 139.99, 150.96, 164.03, 166.89, 168.03.

Compound 14: HRMS (m/z): 482 (M+Na); ¹H NMR (400 MHz, CDCl₃): δ 3.01-3.02 (dd, 1H), 3.45-3.48 (dd, 1H), 3.37-3.86 (m, 16H), 5.63-5.65 (t, 1H), 7.33-7.41 (m, 3H), 758-7.60 (d, 2H), 8.14 (s, 1H); ¹³CNMR (100 MHz, CDCl₃): δ 27.08, 41.52, 43.23, 46.04, 46.46, 53.97, 66.03, 66.06, 66.49, 66.79, 121.81, 128.48, 129.71, 130.89, 134.36, 138.95, 162.95, 163.19, 165.88, 167.25.

Compound 22: HRMS (m/z): 516 (M+Na); ¹H NMR (400 MHz, CDCl₃): δ 3.03-3.04 (dd, 1H), 3.48-3.52 (dd, 1H), 3.37-3.83 (m, 16H), 5.65-5.67 (t, 1H), 7.26-7.28 (d, 2H), 753-7.55 (d, 2H), 8.06 (s, 1H); ¹³CNMR (100 MHz, CDCl₃): δ 27.01, 41.54, 46.06, 53.76, 66.04, 66.47, 122.37, 128.78, 132.06, 132.81, 135.58, 137.21, 162.97, 165.78, 167.16.

Compound 23: HRMS (m/z): 512 (M+Na); ¹H NMR (400 MHz, CDCl₃): δ 3.02-3.03 (dd, 1H), 3.37-3.44 (dd, 1H), 3.57-3.84 (m, 16H), 5.61-5.63 (t, 1H), 6.91-6.93 (d, 2H), 759-7.61 (d, 2H), 8.11 (s, 1H); ¹³CNMR (100 MHz, CDCl₃): δ 27.17, 41.50, 46.03, 54.20, 55.40, 66.07, 113.99, 118.71, 127.19, 133.11, 139.18, 160.78, 163.05, 463.43, 166.04, 167.33

Compound 24: HRMS (m/z): 496 (M+Na); ¹H NMR (400 MHz, CDCl₃): δ 2.37 (s, 3H), 2.98-3.01 (dd, 1H), 3.40-3.57 (dd, 1H), 3.57-3.90 (m, 16H), 5.62-5.64 (t, 1H), 7.20-7.26 (m, 2H), 750-7.52 (d, 2H), 8.13 (s, 1H); ¹³CNMR (100 MHz, CDCl₃): δ 21.56, 27.12, 41.52, 46.04, 54.09, 66.05, 129.24, 131.04, 131.64, 139.33, 140.32, 162.99, 163.23, 165.88, 167.31.

Compound 25: HRMS (m/z): 525 (M+Na); ¹H NMR (400 MHz, CDCl₃): δ 3.00 (s, 6H), 3.38-3.41 (t, 1H), 3.57-3.88 (m, 17H), 5.56-5.58 (t, 1H), 6.66-6.68 (d, 2H), 758-7.60 (d, 2H), 8.11 (s, 1H); ¹³CNMR (100 MHz, CDCl₃): δ 27.41, 40.02, 41.47, 43.19, 46.01, 46.44, 54.70, 66.07, 66.11, 66.50, 111.31, 114.74, 122.32, 133.58, 140.80, 151.17, 163.28, 163.82, 166.35, 167.46.

Compound 26: HRMS (m/z): 527 (M+Na); ¹H NMR (400 MHz, CDCl₃): δ 3.08-3.09 (dd, 1H), 3.34-3.85 (m, 17H), 5.73-5.77 (1, 1H), 7.73-7.75 (d, 2H), 8.20-8.22 (d, 2H), 8.08 (s, 1H); ¹³CNMR (100 MHz, CDCl₃): δ 26.96, 41.58, 43.30, 46.09, 53.25, 66.01, 66.47, 66.81, 123.67, 126.36, 131.20, 134.63, 140.53, 147.31, 162.44, 162.66, 165.49, 166.95.

Compound 27: HRMS (m/z): 521 (M+Na); ¹H NMR (400 MHz, CDCl₃+CCl₄): δ 1.59-1.75 (m, 12H), 2.95-2.98 (d, 1H), 3.01 (s, 6H), 3.27-3.63 (m, 8H), 3.38-3.42 (dd, 1H), 5.59-5.61 (t, 1H), 6.62-6.64 (d, 2H), 753-7.55 (d, 2H), 8.03 (s, 1H); ¹³CNMR (100 MHz, CDCl₃+CCl₄): δ 24.58, 24.73, 24.85, 25.09, 25.55, 26.49, 27.44, 40.04, 41.87, 44.13, 46.82, 46.98, 54.26, 96.15, 111.31, 115.66, 122.88, 133.30, 139.51, 150.77, 163.07, 163.52, 165.61, 167.78.

Compound 28: HRMS (m/z): 512 (M+Na); ¹H NMR (400 MHz, CDCl₃+CCl₄): δ 1.62-1.77 (m, 12H), 2.98-3.03 (dd, 1H), 3.25-3.32 (m, 2H), 3.48-3.66 (m, 7H), 5.69-5.71 (t, 1H), 7.26-7.35 (d, 2H), 751-7.53 (d, 2H), 8.00 (s, 1H); ¹³CNMR (100 MHz, CDCl₃+CCl₄): δ 24.50, 24.66, 24.82, 25.10, 25.52, 26.60, 27.18, 41.97, 44.22, 46.90, 53.38, 96.15, 122.89, 128.63, 131.93, 133.14, 135.13, 135.99, 162.68, 165.09, 167.47.

Compound 29: HRMS (m/z): 523 (M+Na); ¹H NMR (400 MHz, CDCl₃): δ 1.59-1.72 (m, 12H), 3.05-3.10 (dd, 1H), 3.25-3.30 (m, 2H), 3.51-3.65 (m, 7H), 5.79-5.81 (t, 1H), 7.72-7.74 (d, 2H), 8.20-8.23 (d, 2H), 8.04 (s, 1H); ¹³CNMR (100 MHz, CDCl₃): δ 24.34, 24.53, 24.79, 25.13, 25.50, 26.60, 27.19, 42.10, 44.32, 47.02, 47.08, 52.95, 123.60, 126.90, 131.13, 133.63, 140.91, 147.10, 162.40, 162.62, 164.86, 167.37.

Compound 30: HRMS (m/z): 478 (M+Na); ¹H NMR (400 MHz, CDCl₃): δ 1.59-1.77 (m, 12H), 2.99-3.04 (dd, 1H), 3.28-3.36 (m, 2H), 3.47-3.70 (m, 7H), 5.72-5.74 (t, 1H), 7.26-7.40 (m, 3H), 7.58-7.60 (d, 2H), 8.11 (s, 1H); ¹³CNMR (100 MHz, CDCl₃): δ 24.42, 24.58, 24.82, 25.13, 25.51, 26.54, 27.20, 42.03, 44.24, 46.95, 47.04, 53.65, 122.25, 128.38, 129.34, 130.80, 134.67, 137.92, 162.95, 163.12, 165.24, 167.69.

Compound 31: HRMS (m/z): 508 (M+Na); ¹H NMR (400 MHz, CDCl₃): δ 1.56-1.65 (m, 12H), 2.97-3.02 (dd, 1H), 3.22-3.66 (m, 10H), 3.78 (s, 1H), 5.68 (br s, 1H), 6.86-6.88 (d, 2H), 7.55-7.57 (d, 2H), 8.04 (s, 1H); ¹³CNMR (100 MHz, CDCl₃): δ 24.36, 24.40, 24.56, 24.81, 25.10, 25.28, 25.51, 26.38, 26.50, 27.22, 41.70, 41.97, 44.19, 46.90, 47.00, 47.17, 53.86, 55.35, 113.87, 119.35, 127.46, 132.92, 137.93, 160.43, 163.05, 163.40, 163.50, 165.36, 167.70.

Compound 32: HRMS (m/z): 492 (M+Na); ¹H NMR (400 MHz, CDCl₃): δ 1.61-1.73 (m, 12H), 2.99-3.03 (dd, 1H), 3.25-3.36 (m, 2H), 3.46-3.70, (m, 7H), 5.70-5.72 (t, 1H), 7.18-7.20 (d, 2H), 7.49-7.51 (d, 2H), 8.09 (s, 1H); ¹³CNMR (100 MHz, CDCl₃): δ 21.52, 24.43, 24.58, 24.82, 25.12, 25.50, 26.53, 27.23, 42.02, 44.24, 46.95, 53.78, 121.06, 129.13, 130.93, 131.94, 138.25, 139.84, 163.00, 163.25, 165.32, 167.74.

Compound 59: HRMS (m/z): 632 (M+Na); ¹H NMR (400 MHz, CDCl₃): δ 3.07-3.56 (m, 12H), 3.76-3.90 (m, 6H), 5.77 (t, 1H), 6.90-6.97 (m, 6H), 7.27-7.62 (m, 10H), 8.15 (s, 1H); ¹³CNMR (100 MHz, CDCl₃): δ 27.23, 41.17, 45.66, 45.99, 48.79, 48.83, 49.65, 54.03, 116.83, 116.89, 120.49, 121.01, 121.84, 128.47, 129.26, 129.35, 129.64, 130.91, 134.44, 138.82, 150.65, 151.00, 162.90, 163.12, 165.74, 167.35.

Compound 60: HRMS (m/z): 646 (M+Na); ¹H NMR (400 MHz, CDCl₃): δ 2.38 (s, 3H), 3.09 (dd, 1H), 3.24-3.39 (m, 8H), 3.51-3.57 (m, 3H), 3.75-3.90 (m, 6H), 5.75 (t, 1H), 6.89-6.97 (m, 6H), 7.21-7.34 (m, 6H), 7.51 (d, 2H), 8.14 (s, 1H); ¹³CNMR (100 MHz, CDCl₃): δ 21.56, 27.27, 41.15, 45.64, 48.83, 49.57, 54.15, 116.82, 116.88, 120.47, 120.64, 120.98, 129.23, 129.26, 129.35, 131.06, 131.72, 139.13, 140.23, 150.66, 151.01, 162.96, 163.25, 165.82, 167.40.

Compound 61: HRMS (m/z): 677 (M+Na); ¹H NMR (400 MHz, CDCl₃): δ 3.23-3.91 (m, 18H), 5.85 (t, 1H), 6.89-7.04 (m, 6H), 7.27-7.37 (m, 4H), 7.74 (d, 2H), 8.21-8.25 (m, 2H); ¹³CNMR (100 MHz, CDCl₃): δ 27.15, 29.71, 41.23, 43.12, 45.73, 46.02, 48.80, 49.58, 49.92, 53.33, 116.83, 116.90, 120.60, 121.11, 123.66, 126.47, 129.30, 129.38, 131.22, 134.48, 140.61, 147.27, 150.56, 150.93, 162.40, 162.61, 165.36, 167.06.

Compound 62: HRMS (m/z): 675 (M+Na); ¹H NMR (400 MHz, CDCl₃): δ 3.03-3.06 (m, 7H), 3.09-3.58 (m, 11H), 3.76-3.89 (m, 5H), 5.69 (t, 1H), 6.66 (d, 2H), 6.88-6.97 (m, 6H), 7.27-7.33 (m, 4H), 7.59 (d, 2H), 8.11 (s, 1H); ¹³CNMR (100 MHz, CDCl₃): δ 27.53, 40.03, 41.10, 42.92, 45.59, 48.74, 48.81, 49.70, 54.74, 111.33, 114.84, 116.78, 116.84, 120.35, 120.87, 122.41, 129.24, 129.33, 133.57, 140.61, 150.73, 151.07, 151.13, 163.23, 163.76, 166.19, 167.55.

Compound 63: HRMS (m/z): 666 (M+Na); ¹H NMR (400 MHz, CDCl₃): δ 3.22-3.90 (m, 18H), 5.79 (t, 1H), 6.90-7.0 (m, 6H), 7.27-7.46 (m, 6H), 7.53 (d, 2H), 8.0 (s, 1H); ¹³CNMR (100 MHz, CDCl₃): δ 27.17, 41.19, 43.02, 45.67, 45.96, 48.80, 49.59, 49.84, 53.86, 116.83, 116.89, 120.55, 121.03, 122.52, 128.76, 129.28, 129.37, 131.41, 132.10, 132.89, 135.47, 136.97, 150.62, 150.95, 162.87, 162.95, 165.67, 167.23.

Compound 64: HRMS (m/z): 678 (M+Na); ¹H NMR (400 MHz, CDCl₃): 0.8-1.00 (m, 7H), 1.60 (s, 4H), 1.92-2.36 (m, 6H), 3.02-3.52 (m, 12H), 3.76-3.87 (m, 6H), 5.22 (s, 1H), 5.72 (s, 1H), 6.89-6.97 (m, 6H), 7.27-7.36 (m, 5H); ¹³CNMR (100 MHz, CDCl₃): δ12.68, 19.86, 25.88, 26.75, 29.72, 31.01, 35.62, 41.10, 42.99, 45.58, 45.94, 47.00, 48.73, 49.13, 49.79, 54.26, 116.78, 116.85, 120.43, 120.94, 121.57, 122.89, 129.25, 129.34, 144.23, 148.19, 150.68, 151.00, 162.32, 162.95, 165.88, 167.39.

Compound 66: HRMS (m/z): 748 (M+Na); ¹H NMR (400 MHz, CDCl₃): δ 2.46-2.57 (m, 8H), 2.87 (d, 1H), 2.94 (m, 2H), 3.37 (t, 2H), 3.44-3.49 (m, 5H), 3.57-3.73 (m, 6H), 5.68 (t, 1H), 5.92 (d, 4H), 6.74 (d, 4H), 6.84 (t, 2H), 7.34-7.43 (m, 3H), 7.60 (d, 2H), 8.12 (s, 1H).

Compound 67: HRMS (m/z): 762 (M+Na); ¹H NMR (400 MHz, CDCl₃): δ 1.28 (s, 3H), 2.37-2.59 (m, 8H), 2.95 (m, 2H), 3.36-3.72 (m, 12H), 5.66 (t, 1H), 5.94 (d, 4H), 6.74 (t, 4H), 6.84 (d, 2H), 7.19-7.26 (m, 2H), 7.5 (d, 2H), 8.11 (s, 1H); ¹³CNMR (100 MHz, CDCl₃): 14.08, 21.48, 27.09, 29.64, 31.87, 36.45, 41.08, 42.91, 45.65, 45.88, 51.61, 51.80, 52.40, 52.82, 53.80, 62.37, 100.86, 107.84, 107.92, 109.29, 109.40, 120.78, 122.21, 122.24, 128.94, 129.12, 129.88, 131.14, 131.76, 138.71, 139.98, 146.69, 146.82, 147.64, 147.74, 162.86, 163.07, 165.46, 167.36.

Compound 68: HRMS (m/z): 782 (M+Na); ¹H NMR (400 MHz, CDCl₃): δ 2.46-2.58 (m, 8H), 2.88-2.95 (m, 2H), 3.38-3.72 (m, 12H), 5.69 (t, 1H), 5.93 (d, 4H), 6.72-6.86 (m, 6H), 7.27-7.55 (m, 4H), 8.02 (s, 1H); ¹³CNMR (100 MHz, CDCl₃): δ 27.06, 29.70, 31.92, 41.06, 41.84, 42.96, 45.64, 45.90, 51.58, 51.76, 52.39, 52.84, 53.60, 62.41, 100.95, 101.01, 107.93, 108.00, 109.38, 109.52, 122.33, 122.42, 122.67, 128.50, 128.71, 130.91, 131.13, 131.24, 132.02, 132.99, 135.44, 136.67, 146.83, 146.94, 147.93, 147.81, 162.78, 162.83, 165.36, 167.24.

Compound 69: HRMS (m/z): 793 (M+Na); ¹H NMR (400 MHz, CDCl₃): δ 2.45-2.57 (m, 8H), 2.88-3.70 (m, 12H), 5.77 (t, 1H), 5.94 (d, 4H), 6.71-6.86 (m, 6H), 7.73 (d, 2H), 8.07 (s, 1H), 8.23 (d, 2H); ¹³CNMR (100 MHz, CDCl₃): δ 27.07, 29.35, 31.92, 41.26, 43.09, 45.82, 46.01, 51.67, 51.86, 52.43, 52.90, 53.08, 62.41, 62.51, 100.94, 101.01, 107.91, 107.99, 109.30, 109.40, 122.23, 123.18, 123.62, 126.68, 129.91, 131.16, 131.48, 134.11, 140.75, 146.78, 146.91, 147.21, 147.73, 147.82, 162.31, 162.59, 165.07, 167.06.

Compound 70: HRMS (m/z): 791 (M+Na); ¹H NMR (400 MHz, CDCl₃): δ 1.24 (s, 3H), 2.41-2.57 (m, 8H), 2.85-3.00 (m, 8H), 3.38-3.68 (m, 12H), 5.27 (t, 1H), 5.92 (d, 4H), 6.64-6.84 (m, 8H), 7.56 (d, 2H), 8.05 (s, 1H); ¹³CNMR (100 MHz, CDCl₃): δ 14.16, 22.69, 27.36, 29.16, 29.69, 40.01, 41.05, 42.88, 45.63, 45.85, 51.69, 51.86, 52.46, 52.90, 54.49, 62.41, 62.47, 100.92, 100.98, 107.89, 107.96, 109.37, 109.47, 111.31, 115.18, 122.28, 122.49, 131.25, 131.49, 133.45, 140.11, 146.72, 146.84, 147.68, 147.76, 151.03, 163.19, 163.68, 165.88, 167.52.

Compound 71: HRMS (m/z): 794 (M+Na); ¹H NMR (400 MHz, CDCl₃): δ 0.8 (s, 3H), 0.99 (s, 3H), 1.60 (s, 3H), 1.83-2.59 (m, 14H), 2.96 (m, 1H), 3.32-3.67 (m, 12H), 5.21 (s, 1H), 5.62 (t, 1H), 5.94 (d, 3H), 6.72-6.85 (m, 6H), 7.27-7.33 (m, 1H); ¹³CNMR (100 MHz, CDCl₃): δ 12.65, 19.87, 25.81, 26.60, 30.89, 35.54, 41.16, 43.01, 45.71, 46.00, 46.98, 49.18, 51.71, 51.88, 52.47, 52.90, 53.97, 62.46, 62.53, 100.91, 100.99, 107.88, 107.99, 109.30, 109.39, 121.60, 122.20, 123.01, 131.33, 131.70, 143.86, 146.71, 146.85, 147.69, 147.79, 148.16, 162.18, 162.93, 165.56, 167.41.

Compound 88: HRMS (m/z): 604 (M+Na); ¹H NMR (400 MHz, CDCl₃): δ 2.92-3.10 (m, 5H), 3.53-3.89 (m, 8H), 4.63-4.90 (m, 4H), 5.83 (br s, 1H), 6.87 (d, 2H), 7.15-7.26 (m, 8H), 7.53-7.61 (dd, 2H), 8.1 (s, 1H); ¹³CNMR (100 MHz, CDCl₃): δ 27.17, 27.91, 28.37, 28.67, 29.54, 39.34, 43.47, 43.62, 45.26, 47.04, 47.52, 54.44, 55.36, 113.69, 113.94, 119.17, 126.06, 126.41, 126.53, 126.66, 126.82, 127.34, 128.36, 128.49, 128.68, 128.79, 129.14, 131.92, 132.21, 133.05, 133.55, 134.25, 134.49, 138.47, 160.52, 160.59, 163.60, 163.71, 166.38, 167.36, 167.48.

Compound 89: HRMS (m/z): 617 (M+Na); ¹H NMR (400 MHz, CDCl₃): δ 2.92 (s, 1H), 3.48-4.07 (m, 5H), 4.78-4.87 (m, 4H), 5.78 (br s, 1H), 6.64 (d, 2H), 7.16-7.22 (m, 8H), 7.58 (m, 2H), 8.11 (s, 1H).

Compound 90: MS (m/z): 565 (M+); ¹H NMR (400 MHz, CDCl₃): δ 2.36 (s, 3H), 2.93-3.14 (m, 5H), 3.51-3.9 (m, 5H), 4.5-4.9 (m, 4H), 5.84 (br s, 1H), 7.15-7.26 (m, 8H), 7.45-7.52 (m, 4H), 8.11 (s, 1H).

Compound 91: MS (m/z): 551 (M+); ¹H NMR (400 MHz, CDCl₃): δ 2.92-3.13 (m, 5H), 3.54-3.89 (m, 5H), 4.64-4.91 (m, 4H), 5.85 (br s, 1H), 7.16-7.25 (m, 8H), 7.36 (m, 2H), 7.54-7.61 (dd, 2H), 8.13 (s, 1H).

Compound 92: MS (m/z): 585.5 (M+); ¹H NMR (400 MHz, CDCl₃): δ 2.94-3.16 (m, 5H), 3.54-3.90 (m, 5H), 4.55-4.89 (m, 4H), 5.87 (br s, 1H), 7.13-7.26 (m, 8H), 7.31-7.53 (m, 4H), 8.03 (s, 1H).

Compound 93: MS (m/z): 596 (M+); ¹H NMR (400 MHz, CDCl₃): δ 2.95-3.20 (m, 5H), 3.58-3.90 (m, 5H), 4.54-4.88 (m, 4H), 5.93 (br s, 1H), 7.65-7.76 (dd, 2H), 8.05 (s, 1H), 8.18-8.22 (m, 2H).

Compound 94: MS (m/z): 597 (M+); ¹H NMR (400 MHz, CDCl₃): δ 0.76 (m, 3H), 0.95 (m, 3H), 1.58-2.3 (m, 7H), 2.9-3.1 (m, 6H), 3.4-3.8 (m, 6H), 4.5-4.86 (m, 4H), 5.22 (s, 1H), 5.79 (br s, 1H), 7.10-7.28 (m, 8H).

### General Procedure for Synthesis of Compound 33

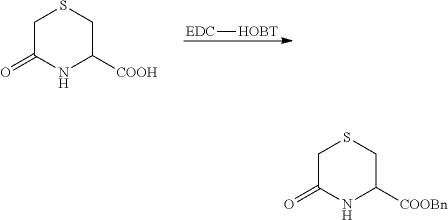

To a solution of the acid (3 mmol) and HOBT (4.5 mmol) in dry DMF (5 ml), at 0° C. was added EDC (4.5 mmol) and the RM stirred at 0° C. for 2 h. The RM was treated with benzyl alcohol (4.5 mmol) and stirred at RT for 17 h. The RM was then treated with dil. HCl and extracted with ethyl acetate. The organic layers were combined and washed with NaHCO₃ followed by brine and dried over sodium sulfate. The solvent was removed under vacuum to afford the crude, which was purified using n-hexane/ethyl acetate mixtures on a silica gel column.

Compound 33: HRMS (m/z): 251 (M+); ¹H NMR (400 MHz, CDCl₃): δ 2.92-3.10 (m, 2H), 3.22-3.33 (m, 2H, 4.39 (t, 1H), 5.15-5.24 (q, 2H), 7.11 (s, 1H), 7.26-7.37 (m, 5H); ¹³CNMR (100 MHz, CDCl₃): δ 27.43, 29.60, 56.97, 67.95, 128.46, 128.76, 134.84, 166.62, 169.21.

### General Procedure for Synthesis of Compounds 8, 15, 57, 72, 80, 34 & 42

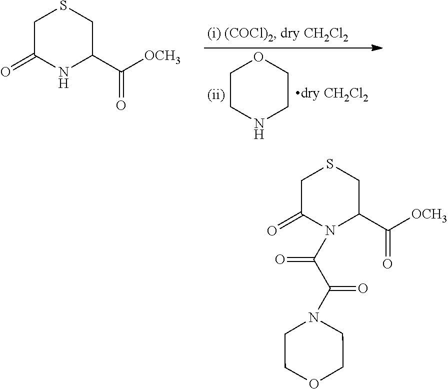

To a solution of the ester (1 mmol) in 5 ml of dry dichloromethane was added oxalyl chloride (3 mmol) and the RM was stirred at RT for 3 h. The RM was completely dried under vacuum and dry dichloromethane (5 ml) was added to the crude and cooled to 0° C. The desired amine (3 mmol) was added, stirred for 2 h at 0° C. and an additional 2 h at RT. The RM was diluted with dichloromethane and washed with small volumes of 3N HCl, saturated NaHCO₃ solution and brine. Organic layer was dried over anhydrous Na₂SO₄ and then concentrated to get the crude product, which was purified using a silica gel column. Compounds 34 and 42 were synthesized starting from Compound 33.

Compound 8: HRMS (m/z): 325 (M+Na); ¹H NMR (400 MHz, CDCl₃+CCl₄): δ 1.14-1.18 (m, 6H), 3.18-3.26 (m, 6H), 3.59-3.63 (m, 2H), 3.78 (s, 3H), 5.30 (s, 1H); ¹³CNMR (100 MHz, CDCl₃+CCl₄): δ 11.67, 13.09, 27.36, 31.60, 38.37, 41.85, 53.11, 57.57, 163.00, 166.25, 166.92, 168.73.

Compound 15: HRMS (m/z): 339 (M+Na); ¹H NMR (400 MHz, CDCl₃): δ 3.28-3.56 (m, 4H), 3.56-3.78 (m, 8H), 3.82 (s, 3H), 5.36 (t, 1H); ¹³CNMR (75 MHz, CDCl₃): δ 27.35, 31.76, 41.52, 45.99, 53.33, 57.82, 65.91, 162.41, 166.70, 168.84.

Compound 34: HRMS (m/z): 392 (M+); ¹H NMR (400 MHz, CDCl₃): δ 3.14-4.00 (m, 12H), 5.11-5.53 (m, 3H), 7.29-7.36 (m, 5H); ¹³CNMR (100 MHz, CDCl₃): δ 27.52, 31.93, 41.50, 45.94, 58.11, 65.80, 65.93, 68.41, 128.51, 128.80, 128.91, 134.52, 162.44, 166.80, 168.34, 168.54.

Compound 42: MS (m/z): 467 (M+); ¹H NMR (400 MHz, CDCl₃): δ 3.02-3.08 (m, 2H), 3.21-3.40 (m, 7H), 3.40 (d, 2H), 3.68 (m, 1H), 5.16 (d, 1H), 5.30 (d, 1H), 5.42 (s, 1H), 6.92 (d, 3H), 7.26-7.41 (m, 8H); ¹³CNMR (100 MHz, CDCl₃): δ 27.52, 31.91, 41.09, 45.51, 48.54, 48.68, 58.06, 116.83, 120.57, 128.35, 128.55, 128.81, 128.88, 129.24, 134.55, 150.87, 162.29, 166.22, 166.88, 168.38.

Compound 57: HRMS (m/z): 457 (M+Na); ¹H NMR (400 MHz, CDCl₃): δ 2.37 (s, 3H), 3.36-3.44 (m, 4H), 3.71-3.82 (m, 6H), 3.78 (s, 3H), 5.57-5.59 (t, 1H), 7.21-7.26 (d, 2H), 7.50-7.52 (d, 2H), 8.05 (s, 1H); ¹³CNMR (100 MHz, CDCl₃): δ 21.56, 26.93, 41.54, 45.99, 53.48, 53.46, 66.01, 66.05, 119.99, 129.31, 131.00, 131.58, 139.31, 140.44, 162.22, 162.86, 166.76, 168.40.

Compound 72: HRMS (m/z): 414 (M+Na); ¹H NMR (400 MHz, CDCl₃+CCl₄): δ 3.21-3.85 (m, 6H), 3.53-3.85 (m, 7H), 5.36 (s, 1H), 6.86-6.93 (q, 3H), 7.24-7.27 (t, 2H); ¹³CNMR (100 MHz, CDCl₃+CCl₄): δ 27.35, 31.75, 41.13, 45.60, 48.81, 53.37, 57.86, 116.93, 120.62, 129.22, 150.95, 162.27, 166.55, 166.79, 168.87.

Compound 80: HRMS (m/z): 450 (M+H); ¹H NMR (400 MHz, CDCl₃+CCl₄): δ 2.44-2.56 (br m, 4H), 2.86 (d, 1H), 2.95 (d, 1H), 3.24-3.79 (m, 12H), 5.32 (s, 1H), 5.92 (d, 2H), 6.7 (s, 2H), 6.82 (s, 1H); ¹³CNMR (100 MHz, CDCl₃+CCl₄): δ 27.33, 31.71, 41.12, 45.68, 51.72, 53.30, 57.75, 62.52, 100.87, 107.89, 109.35, 122.20, 131.38, 146.79, 147.94, 162.16, 166.43, 166.80, 168.80.

### General Procedure for Synthesis of Compounds 16-21, 35-41, 43-48, 51-56, 73-79, 81-85

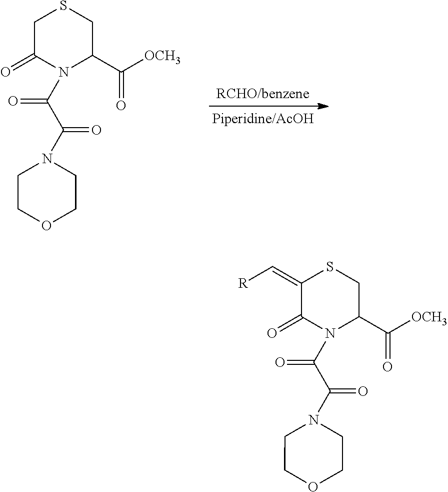

To a solution of amide (1 mmol) in 5 ml of dry benzene were added piperidine (80 mmol) and acetic acid (80 mmol) followed by aldehyde (1.5 mmol). The RM was refluxed at 100° C. using a Dean-Stark set up for 1 h. After completion of the reaction, the solvents were removed from the reaction mixture and ethyl acetate was added to the crude. The mixture was washed with small volumes of 3N HCl and with saturated NaHCO₃ solution followed by brine. Organic layer was dried over anhydrous Na₂SO₄ and then concentrated to get the crude product, which was purified on a silica gel column.

Compound 16: HRMS (m/z): 427 (M+Na); ¹H NMR (400 MHz, CDCl₃): δ 3.38-3.46 (m, 4H), 3.72-3.86 (m, 6H), 3.80 (s, 3H), 5.60-5.61 (t, 1H), 7.37-7.44 (m, 3H), 7.60-7.62 (d, 2H), 8.07 (s, 1H); ¹³CNMR (100 MHz, CDCl₃): δ 26.91, 41.56, 45.99, 53.52, 53.41, 66.00, 66.04, 121.26, 128.56, 129.81, 130.84, 134.82, 139.06, 162.11, 162.81, 166.69, 168.37.

Compound 17: HRMS (m/z): 457 (M+Na); ¹H NMR (400 MHz, CDCl₃): δ 2.37 (s, 3H), 3.36-3.44 (m, 4H), 3.71-3.82 (m, 6H), 3.78 (s, 3H), 5.57-5.59 (t, 1H), 7.21-7.26 (d, 2H), 7.50-7.52 (d, 2H), 8.05 (s, 1H); ¹³CNMR (100 MHz, CDCl₃): δ 21.56, 26.93, 41.54, 45.99, 53.48, 53.46, 66.01, 66.05, 119.99, 129.31, 131.00, 131.58, 139.31, 140.44, 162.22, 162.86, 166.76, 168.40.

Compound 18: HRMS (m/z): 461 (M+Na); ¹H NMR (400 MHz, CDCl₃): δ 3.37-3.49 (m, 4H), 3.72-3.87 (m, 6H), 3.84 (s, 3H), 5.61-5.63 (t, 1H), 7.38-7.40 (d, 2H), 7.54-7.57 (d, 2H), 8.00 (s, 1H); ¹³CNMR (100 MHz, CDCl₃): δ 26.90, 41.57, 46.01, 53.56, 55.27, 60.41 66.00, 66.04, 121.85, 128.85, 132.02, 132.71, 135.70, 137.43, 161.91, 162.71, 166.62, 168.30.

Compound 19: HRMS (m/z): 441 (M+Na); ¹H NMR (400 MHz, CDCl₃): δ 3.35-3.45 (m, 4H), 3.65-3.77 (m, 6H), 3.82 (s, 3H), 3.83 (s, 1H), 5.57-5.59 (t, 1H), 6.92-6.94 (d, 2H), 7.60-7.62 (d, 2H), 8.04 (s, 1H); ¹³CNMR (100 MHz, CDCl₃): δ 26.93, 41.52, 45.97, 53.45, 55.41, 55.51, 60.39, 66.00, 66.04, 114.06, 118.09, 127.12, 133.08, 139.11, 160.82, 162.32, 162.92, 166.78, 168.43.

Compound 20: HRMS (m/z): 472 (M+Na); ¹H NMR (300 MHz, CDCl₃): 3.36-3.44 (m, 4H), 3.72-3.86 (m, 6H), 3.80 (s, 3H), 5.64-5.66 (t, 1H), 7.74-7.77 (d, 2H), 8.25-8.28 (d, 2H), 8.04 (s, 1H).

Compound 21: HRMS (m/z): 470 (M+Na); ¹H NMR (400 MHz, CDCl₃): δ 3.01-3.04 (s, 6H), 3.36-3.44 (m, 4H), 3.66-3.82 (m, 9H), 5.56 (t, 1H), 6.67-6.69 (d, 1H), 7.58-7.6 (d, 2H), 8.05 (s, 1H); ¹³CNMR (100 MHz, CDCl₃): δ 27.06, 29.70, 40.02, 41.50, 45.97, 53.35, 55.79, 66.06, 111.35, 122.32, 133.54, 140.58, 151.18, 162.59, 163.17, 166.98, 168.59.

Compound 35: HRMS (m/z): 517 (M+Na); ¹H NMR (400 MHz, CDCl₃): δ 2.38 (s, 3H), 3.27-3.83 (m, 10H), 5.14-5.26 (m, 2H), 5.63 (t, 1H), 7.21 (d, 2H), 7.30 (m, 5H), 7.52 (d, 2H), 8.02 (s, 1H); ¹³CNMR (100 MHz, CDCl₃): δ 21.40, 26.86, 41.37, 45.75, 55.29, 65.70, 65.83, 120.02, 128.31, 128.55, 128.64, 128.96, 129.95, 130.86, 131.47, 134.38, 138.92, 140.25, 162.14, 162.70, 166.50, 167.59.

Compound 36: HRMS (m/z): 533 (M+Na); ¹H NMR (400 MHz, CDCl₃): δ 3.26-3.82 (m, 10H), 3.84 (s, 3H), 5.14-5.28 (m, 2H), 5.64 (t, 1H), 6.96 (d, 2H), 7.31 (m, 5H), 7.62 (d, 2H), 8.01 (s, 1H); ¹³CNMR (100 MHz, CDCl₃): δ 27.03, 41.47, 45.86, 55.43, 65.83, 65.96, 68.36, 114.06, 118.22, 127.14, 128.41, 128.65, 128.73, 133.03, 134.52, 138.88, 160.83, 162.37, 162.85, 166.72, 167.74.

Compound 37: HRMS (m/z): 514.5 (M+); ¹H NMR (400 MHz, CDCl₃): δ 3.26-3.88 (m, 10H), 5.18-5.30 (m, 2H), 5.68 (s, 1H), 7.28-7.42 (m, 9H), 7.54 (d, 2H), 7.98 (s, 1H); ¹³CNMR (100 MHz, CDCl₃): δ 26.54, 41.07, 45.46, 54.74, 65.43, 65.54, 68.12, 121.47, 127.91, 128.09, 128.28, 128.29, 128.41, 131.59, 137.24, 133.94, 135.24, 136.81, 161.52, 166.12, 167.19.

Compound 38: MS (m/z): 480 (M+); ¹H NMR (400 MHz, CDCl₃): δ 3.26-3.87 (m, 10H), 5.18-5.30 (dd, 2H), 5.67 (s, 1H), 7.29-7.48 (m, 9H), 7.61 (d, 2H), 8.0 (s, 1H); ¹³CNMR (100 MHz, CDCl₃): δ 26.55, 41.05, 45.45, 54.88, 65.43, 65.55, 68.07, 120.84, 127.91, 128.06, 128.13, 128.28, 128.41, 129.40, 130.42, 133.82, 133.98, 138.52, 161.72, 162.33, 166.21, 167.26.

Compound 39: HRMS (m/z): 546 (M+Na); ¹H NMR (400 MHz, CDCl₃): δ 3.02 (s, 6H), 3.21-3.82 (m, 10H), 5.14-5.26 (m, 2H), 5.60 (t, 1H), 6.68 (d, 2H), 7.32 (m, 5H), 7.60 (d, 2H), 8.01 (s, 1H); ¹³CNMR (100 MHz, CDCl₃): δ 27.14, 40.05, 41.48, 45.89, 55.80, 65.90, 66.03, 68.32, 111.41, 122.33, 128.38, 128.46, 128.73, 128.76, 135.58, 134.66, 140.41, 151.23, 162.68, 163.20, 166.94, 168.0.

Compound 40: MS (m/z): 525 (M+); ¹H NMR (400 MHz, CDCl₃): δ 3.29-3.86 (m, 10H), 5.20-5.31 (dd, 2H), 5.71 (t, 1H), 7.29-7.38 (m, 9H), 7.71 (d, 2H), 8.00 (s, 1H), 8.27 (d, 2H); ¹³CNMR (100 MHz, CDCl₃): δ26.58, 41.13, 45.52, 54.45, 65.44, 65.54, 68.24, 123.27, 125.56, 127.89, 128.12, 128.29, 128.50, 130.70, 133.88, 134.56, 139.84, 146.96, 161.04, 162.00, 165.90, 167.01.

Compound 41: MS (m/z): 526 (M+); ¹H NMR (400 MHz, CDCl₃): δ 0.82 (s, 3H), 1.01 (s, 3H), 1.62 (s, 3H), 1.87-2.30 (m, 6H), 3.20-3.81 (m, 10H), 5.15-5.28 (m, 2H), 5.60 (s, 1H), 7.25-7.38 (m, 5H); ¹³CNMR (100 MHz, CDCl₃): δ 12.20, 19.43, 25.38, 26.00, 30.48, 30, 56, 35.07, 35.15, 40.99, 45.39, 46.55, 48.64, 48.75, 55.21, 65.37, 65.50, 67.93, 120.97, 121.10, 122.04, 127.88, 128.00, 128.27, 128.38, 134.05, 144.21, 147.67, 160.88, 162.35, 166.26, 167.37.

Compound 43: HRMS (m/z): 590 (M+H); ¹H NMR (400 MHz, CDCl₃): δ 3.09-3.48 (m, 8H), 3.87-3.90 (m, 2H), 5.19-5.30 (m, 2H), 5.69 (t, 1H), 6.92 (d, 3H), 7.26-7.40 (m, 11H), 7.52 (d, 2H), 7.95 (s, 1H); ¹³CNMR (100 MHz, CDCl₃): δ 27.01, 41.11, 45.53, 48.64, 48.70, 55.19, 68.54, 116.80, 120.56, 121.96, 128.34, 128.56, 128.74, 128.83, 129.26, 132.02, 132.72, 134.46, 135.64, 137.17, 150.89, 161.85, 162.53, 166.67, 167.67.

Compound 44: HRMS (m/z): 570 (M+H); ¹H NMR (400 MHz, CDCl₃): δ 2.40 (s, 3H), 3.06-3.49 (m, 8H), 3.78-3.91 (m, 2H), 5.20-5.30 (m, 2H), 5.68 (t, 1H), 6.92 (d, 3H), 7.24-7.38 (m, 11H), 7.52 (d, 2H), 8.03 (s, 1H); ¹³CNMR (100 MHz, CDCl₃): δ 21.58, 27.03, 41.08, 45.49, 48.66, 55.41, 68.47, 116.79, 120.12, 120.50, 128.34, 128.54, 128.80, 129.25, 129.31, 131.01, 131.60, 134.53, 139.11, 140.40, 150.92, 162.16, 162.69, 166.82, 167.80.

Compound 45: MS (m/z): 598 (M+); ¹H NMR (400 MHz, CDCl₃): δ 3.05 (s, 6H), 3.25-3.43 (m, 6H), 3.79-3.88 (m, 2H), 5.20-5.30 (m, 2H), 5.66 (t, 1H), 6.72 (d, 2H), 6.94 (d, 3H), 7.30-7.39 (m, 10H), 7.62 (d, 2H), 8.05 (s, 1H); ¹³CNMR (100 MHz, CDCl₃): δ 27.13, 40.02, 41.03, 45.44, 48.59, 55.79, 68.31, 111.38, 114.02, 116.76, 120.40, 122.35, 128.35, 128.36, 128.51, 128.73, 129.24, 133.55, 134.68, 140.38, 150.98, 151.17, 162.54, 163.04, 167.05, 168.04.

Compound 46: MS (m/z): 585 (M+); ¹H NMR (400 MHz, CDCl₃): δ 3.10-3.47 (m, 8H), 3.78-3.86 (m, 5H), 5.19-5.30 (q, 2H), 5.67 (t, 1H), 6.91-6.96 (m, 5H), 7.26-7.37 (m, 10H), 7.60 (d, 2H), 8.01 (s, 1H); ¹³CNMR (100 MHz, CDCl₃): δ 27.04, 41.06, 45.48, 48.65, 55.41, 55.47, 68.43, 114.05, 116.77, 118.17, 120.48, 127.16, 128.33, 128.51, 128.72, 128.78, 129.23, 133.07, 134.54, 138.96, 150.92, 160.81, 162.25, 162.75, 166.85, 167.83.

Compound 47: HRMS (m/z): 578 (M+Na); ¹H NMR (400 MHz, CDCl₃): δ 3.06-3.46 (m, 8H), 3.78-3.88 (m, 2H), 5.19-5.30 (dd, 2H), 5.68 (t, 1H), 6.94 (d, 3H), 7.26-7.45 (m, 11H), 7.45 (d, 2H), 8.03 (s, 1H); ¹³CNMR (100 MHz, CDCl₃): δ 27.02, 41.09, 45.50, 48.67, 55.53, 68.51, 116.80, 120.53, 126.96, 127.59, 128.34, 128.54, 128.73, 128.82, 129.25, 129.78, 130.85, 134.29, 134.49, 138.88, 150.91, 162.05, 162.63, 166.76, 167.75.

Compound 48: MS (m/z): 600 (M+); ¹H NMR (400 MHz, CDCl₃): δ 3.13-3.53 (m, 8H), 3.70-3.87 (m, 2H), 5.19-5.31 (q, 2H), 5.72 (t, 1H), 6.92 (d, 3H), 7.28-7.36 (m, 10H), 7.70 (d, 2H), 7.97 (s, 1H), 8.24 (d, 2H); ¹³CNMR (100 MHz, CDCl₃): δ 27.04, 41.16, 45.59, 48.65, 48.77, 54.86, 68.59, 116.81, 120.65, 123.69, 125.97, 126.89, 128.32, 128.60, 128.74, 128.92, 129.27, 131.12, 134.33, 134.99, 147.37, 150.85, 161.34, 162.28, 166.44, 167.48.

Compound 73: HRMS (m/z): 502 (M+Na); ¹H NMR (400 MHz, CDCl₃): δ 3.24-3.58 (m, 8H), 3.82-3.88 (m, 5H), 5.63 (t, 1H), 6.89-6.97 (m, 3H), 7.26-7.3 (t, 2H), 7.36-7.43 (m, 3H), 7.60 (d, 2H), 8.07 (s, 1H); ¹³CNMR (100 MHz, CDCl₃): δ 26.95, 41.22, 45.65, 48.84, 48.96, 53.36, 55.48, 116.94, 120.68, 121.35, 128.44, 128.58, 129.32, 129.81, 130.10, 130.89, 133.39, 134.35, 139.03, 150.96, 162.09, 162.78, 166.81, 168.48.

Compound 74: HRMS (m/z): 516 (M+Na); ¹H NMR (400 MHz, CDCl₃): δ 2.40 (s, 3H), 3.24-3.58 (m, 8H), 3.82-3.88 (m, 5H), 5.62 (t, 1H), 6.89-6.97 (m, 3H), 7.23 (d, 2H), 7.29 (t, 2H), 7.50 (d, 2H), 8.0 (s, 1H); ¹³CNMR (100 MHz, CDCl₃): δ 21.60, 26.98, 41.19, 45.64, 48.82, 48.93, 53.53, 55.51, 116.91, 120.08, 120.62, 129.31, 129.33, 131.05, 131.65, 139.28, 140.43, 151.00, 162.19, 162.80, 166.90, 168.50.

Compound 75: HRMS (m/z): 536 (M+Na); ¹H NMR (400 MHz, CDCl₃): δ 3.24-3.57 (m, 7H), 3.86 (s, 4H), 5.64 (t, 1H), 6.89-6.96 (m, 2H), 7.26-7.3 (t, 2H), 7.38 (d, 1H), 7.52 (d, 2H), 7.97 (s, 1H); ¹³CNMR (100 MHz, CDCl₃): δ 26.94, 41.22, 45.67, 48.80, 48.94, 53.60, 55.33, 116.92, 120.68, 128.74, 128.86, 129.32, 131.44, 132.06, 132.78, 135.68, 137.36, 150.95, 161.87, 162.65, 166.74, 168.40.

Compound 76: HRMS (m/z): 547 (M+Na); ¹H NMR (400 MHz, CDCl₃): δ 3.24-3.57 (m, 7H), 3.86 (s, 4H), 5.64 (t, 1H), 6.89-6.96 (m, 2H), 7.26-7.3 (t, 2H), 7.38 (d, 1H), 7.52 (d, 2H), 7.97 (s, 1H); ¹³CNMR (100 MHz, CDCl₃): δ 26.94, 41.22, 45.67, 48.80, 48.94, 53.60, 55.33, 116.92, 120.68, 128.74, 128.86, 129.32, 131.44, 132.06, 132.78, 135.68, 137.36, 150.95, 161.87, 162.65, 166.74, 168.40.

Compound 77: HRMS (m/z): 545 (M+Na); ¹H NMR (400 MHz, CDCl₃): δ 3.02 (s, 6H) 3.22-3.58 (m, 8H), 3.82-3.87 (m, 5H), 5.59 (t, 1H), 6.68 (d, 2H), 6.88-6.96 (m, 3H), 7.28 (t, 2H), 7.60 (d, 2H), 8.0 (s, 1H); ¹³CNMR (100 MHz, CDCl₃): δ 27.09, 40.05, 41.13, 45.59, 48.82, 48.88, 53.40, 55.83, 111.38, 116.87, 120.50, 122.37, 129.28, 135.57, 140.53, 151.06, 151.18, 162.55, 163.10, 167.11, 168.69.

Compound 78: HRMS (m/z): 548 (M+Na); ¹H NMR (400 MHz, CDCl₃): δ 0.8 (s, 3H), 1.0 (s, 1H), 1.6-2.3 (m, 12H), 3.21-3.52 (m, 8H), 3.8 (s, 5H), 5.58 (s, 1H), 6.88-6.95 (m, 3H), 7.23-7.30 (m, 3H); ¹³CNMR (100 MHz, CDCl₃): δ 12.66, 19.89, 25.84, 26.46, 29.73, 30.97, 35.55, 41.14, 45.59, 47.02, 48.78, 48.88, 49.10, 49.21, 53.46, 55.67, 116.88, 120.59, 121.47, 121.58, 129.28, 144.78, 148.15, 151.00, 161.28, 162.75, 166.89, 168.58.

Compound 79: HRMS (m/z): 532 (M+Na); ¹H NMR (400 MHz, CDCl₃): δ 3.23-3.57 (m, 8H), 3.81-3.88 (m, 5H), 5.61 (t, 1H), 6.88-6.96 (m, 5H), 7.28 (t, 2H), 7.60 (d, 2H), 8.0 (s, 1H); ¹³CNMR (100 MHz, CDCl₃): δ 26.99, 41.17, 45.63, 48.81, 48.91, 53.50, 55.44, 55.56, 60.45, 114.09, 116.88, 120.58, 127.20, 129.30, 133.11, 139.12, 151.01, 160.84, 162.27, 162.84, 166.95, 168.53.

Compound 81: HRMS (m/z): 574 (M+Na); ¹H NMR (400 MHz, CDCl₃): δ 2.38-2.65 (m, 8H), 3.33-3.47 (m, 6H), 3.69 (br s, 2H), 3.81 (s, 2H), 5.59 (t, 1H), 5.93 (s, 2H), 6.74 (s, 2H), 6.86 (s, 1H), 7.21 (d, 2H), 7.52 (d, 2H), 8.0 (s, 1H); ¹³CNMR (100 MHz, CDCl₃): δ 21.59, 27.00, 29.73, 41.25, 45.72, 51.75, 51.90, 53.45, 55.41, 62.55, 100.97, 107.94, 109.41, 122.25, 129.31, 130.41, 130.99, 131.60, 131.69, 139.17, 140.35, 146.79, 147.76, 162.05, 162.70, 166.96, 168.42.

Compound 82: HRMS (m/z): 594 (M+Na); ¹H NMR (400 MHz, CDCl₃): δ 2.46-3.64 (m, 5H), 3.35-3.48 (m, 6H), 3.68-3.84 (m, 5H), 5.61 (t, 1H), 5.93 (s, 2H), 6.74 (s, 2H), 6.86 (s, 2H), 7.39 (t, 2H), 7.54 (d, 2H), 8.0 (s, 1H); ¹³CNMR (100 MHz, CDCl₃): δ 26.96, 41.18, 45.65, 51.66, 51.80, 53.54, 55.23, 62.47, 100.99, 107.97, 109.48, 122.37, 128.37, 128.57, 128.85, 131.27, 132.02, 132.82, 135.63, 137.26, 146.86, 147.78, 161.76, 162.56, 166.79, 168.32.

Compound 83: HRMS (m/z): 603 (M+Na); ¹H NMR (400 MHz, CDCl₃): δ 2.44-2.63 (m, 5H), 3.02 (s, 6H), 3.34-3.46 (m, 6H), 3.68-3.82 (m, 5H), 5.56 (t, 1H), 5.92 (s, 2H), 6.6-6.7 (m, 4H), 6.86 (s, 1H), 7.58 (d, 2H), 8.02 (s, 1H); ¹³CNMR (100 MHz, CDCl₃): δ 27.09, 40.05, 41.20, 45.68, 51.79, 51.92, 53.33, 55.75, 62.55, 100.96, 107.93, 109.42, 111.38, 122.25, 122.41, 131.68, 133.50, 140.36, 146.76, 147.74, 151.15, 162.44, 163.03, 167.16, 168.62.

Compound 84: HRMS (m/z): 584 (M+H); ¹H NMR (400 MHz, CDCl₃): δ 0.79 (s, 3H), 0.98 (s, 3H), 1.59 (s, 3H), 1.87-2.5 (m, 10H), 3.30-3.45 (m, 6H), 3.82 (s, 3H), 3.66 (br s, 2H), 5.20 (s, 1H), 5.55 (t, 1H), 5.9 (s, 2H), 6.7 (s, 2H), 6.85 (s, 1H), 7.24 (t, 1H); ¹³CNMR (100 MHz, CDCl₃): δ 12.67, 19.88, 25.85, 26.46, 30.94, 35.55, 41.20, 45.66, 47.01, 49.13, 49.24, 51.73, 51.85, 53.38, 55.57, 62.53, 100.96, 107.93, 109.40, 121.47, 121.58, 122.24, 131.59, 144.50, 146.78, 147.75, 148.18, 161.18, 162.69, 166.93, 168.51.

Compound 85: HRMS (m/z): 590 (M+Na); ¹H NMR (400 MHz, CDCl₃): δ 2.44-2.55 (m, 4H), 3.38-3.47 (m, 4H), 3.68-3.84 (m, 10H), 5.59 (t, 1H), 5.93 (s, 2H), 6.74 (s, 2H), 6.86 (s, 2H), 6.9 (d, 2H), 7.59 (d, 2H), 8.0 (s, 1H); ¹³CNMR (100 MHz, CDCl₃): δ 27.01, 41.24, 45.71, 51.76, 51.90, 53.43, 55.37, 55.44, 62.56, 100.96, 107.94, 109.42, 113.91, 114.07, 122.25, 127.25, 131.62, 132.26, 133.05, 134.75, 138.99, 146.78, 147.75, 160.79, 162.15, 162.78, 167.00, 168.47.

### General Procedure for Synthesis of Compounds 3, 49, 50, 86, 95

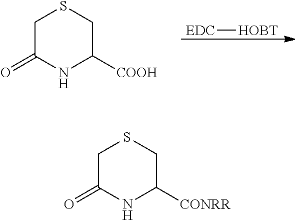

To a solution of the acid (3 mmol) and HOBT (4.5 mmol) in dry DMF (5 ml), at 0° C. was added EDC (4.5 mmol) and the RM stirred at 0° C. for 2 h. The RM was treated with amine (4.5 mmol) and stirred at RT for 17 h. The RM was then treated with dil. HCl and extracted with ethyl acetate. The organic layers were combined and washed with NaHCO₃ followed by brine and dried over sodium sulfate. The solvent was removed under vacuum to afford the crude, which was purified using n-hexane/ethyl acetate mixtures on a silica gel column.

Compound 49: HRMS (m/z): 216 (M+); ¹H NMR (400 MHz, CDCl₃): δ 1.02-1.06 (m, 3H), 1.14-1.18 (m, 3H), 2.72-2.87 (m, 2H), 3.16-3.43 (m, 6H), 4.35 (d, 1H), 6.85 (s, 1H); ¹³CNMR (100 MHz, CDCl₃): δ 12.77, 14.56, 29.02, 29.56, 36.52, 40.78, 41.67, 53.89, 167.14, 167.73.

Compound 50: HRMS (m/z): 228 (M+); ¹H NMR (400 MHz, CDCl₃): δ 1.47-1.56 (m, 6H), 2.64-2.87 (m, 2H), 3.11-3.69 (m, 6H), 4.40 (d, 1H), 6.84 (s, 1H); ¹³CNMR (100 MHz, CDCl₃): δ 24.21, 25.38, 26.43, 29.00, 29.37, 43.83, 46.44, 53.50, 166.0, 167.57.

Compound 86: HRMS (m/z): 277 (M+H); ¹H NMR (400 MHz, CDCl₃): δ 1.89 (d, 1H), 2.06-2.19 (m, 4H), 3.20-3.40 (m, 3H), 3.8 (br s, 1H), 4.94 (d, 1H), 6.94 (s, 1H), 7.0-7.41 (m, 4H); ¹³CNMR (100 MHz, CDCl₃): δ 23.87, 26.32, 28.14, 28.97, 43.80, 54.88, 123.70, 126.73, 126.91, 128.22, 129.00, 137.70, 167.34, 168.00.

Compound 95: HRMS (m/z): 277 (M+H); ¹H NMR (300 MHz, CDCl₃): δ 2.75-3.00 (m, 4H), 3.24-3.38 (m, 2H), 3.70-3.94 (m, 4H), 4.57-4.74 (m, 2H), 7.01 (s, 1H), 7.11-7.34 (m, 4H).

### General Procedure for Synthesis of Compound 87 from Compound 86

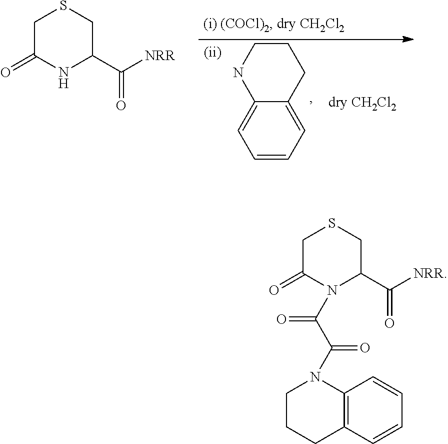

To a solution of the amide (1 mmol) in 5 ml of dry dichloromethane was added oxalyl chloride (3 mmol) and the reaction mixture was stirred at RT for 3 h. The RM was completely dried under vacuum and dry dichloromethane (5 ml) was added to the crude and cooled to 0° C. Then the desired amine (3 mmol) was added and stirred for 2 h at 0° C. and an additional 2 h at RT. The reaction mixture was diluted with dichloromethane and washed with small volumes of 3N HCl, saturated NaHCO₃ solution and brine. Organic layer was dried over anhydrous Na₂SO₄ and concentrated to get the crude product, which was purified on a silica gel column.

Compound 87: HRMS (m/z): 486 (M+Na); ¹H NMR (400 MHz, CDCl₃): δ 2.92-3.01 (m, 5H), 3.33-3.75 (m, 7H), 4.52-4.84 (m, 4H), 5.66 (br s, 1H), 7.13-7.26 (m, 8H); ¹³CNMR (100 MHz, CDCl₃): δ 27.61, 27.79, 28.35, 28.57, 29.42, 32.21, 39.31, 41.17, 43.43, 43.60, 45.19, 47.05, 47.37, 55.41, 55.56, 126.07, 126.36, 126.45, 126.54, 126.61, 126.67, 126.79, 127.42, 128.36, 128.48, 128.75, 129.08, 131.76, 131.95, 133.60, 134.15, 134.36, 162.95, 163.16, 167.31, 168.42, 168.59.

### General Procedure for Sulfide Oxidation to Sulfoxides 96-100 Using Ozone

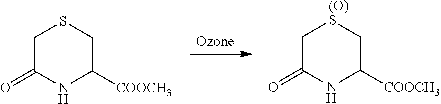

Ozone was passed through a pre-cooled solution of thiomorpholine ester (1 mmol) in 1:5 mixture of MeOH:CH₂Cl₂ (10 mL) and catalytic amount of NaHCO₃ at −70° C. for 10 min. The RM was then purged with O₂ and filtered and evaporated. The crude obtained was purified on a silica gel column.

Compound 96: HRMS (m/z): 214 (M+Na); ¹H NMR (400 MHz, CDCl₃): δ 2.78 (t, 1H), 3.49-3.53 (m, 3H), 3.82 (s, 3H), 4.94-4.97 (d, 1H), 7.18 (br s, 1H); ¹³CNMR (100 MHz, CDCl₃): δ 44.81, 47.12, 49.87, 53.62, 162.62, 170.08.

Compound 97: HRMS (m/z): 214 (M+Na); ¹H NMR (400 MHz, DMSO-d₆): δ 3.20-3.42 (m, 3H), 3.57 (s, 3H), 3.66-3.71 (d, 1H), 4.51-4.52 (m, 1H), 8.3 (br s, 1H); ¹³CNMR (100 MHz, DMSO-d₆): δ 43.55, 49.33, 50.17, 52.57, 162.56, 171.10.

Compound 98: MS (m/z): 266 (M+); ¹H NMR (400 MHz, DMSO-d₆): δ 2.48 (s, 1H), 3.54-3.60 (q, 1H), 3.71-3.74 (dd, 1H), 4.01-4.06 (dd, 1H), 4.27-4.35 (m, 4H), 7.21-7.33 (m, 5H), 8.44 (s, 1H), 8.71 (t, 1H); ¹³CNMR (100 MHz, DMSO-d₆): δ 43.03, 49.24, 52.19, 56.17, 127.38, 127.78, 128.73, 139.01, 163.21, 168.04.

Compound 100: HRMS (m/z): 422 (M+K); ¹H NMR (400 MHz, CDCl₃): δ 1.60-1.68 (m, 12H), 3.21 (br s, 2H), 3.46-3.60 (m, 7H), 3.82-3.87 (m, 1H), 4.05-4.21 (m, 1H), 4.42 (d, 1H), 5.82 (t, 1H); ¹³CNMR (100 MHz, CDCl₃): δ 24.26, 24.37, 24.68, 25.06, 25.21, 26.22, 42.17, 46.62, 46.94, 49.13, 51.57, 58.85, 60.40, 161.52, 163.80, 164.27, 163.00.

### General Procedure for Oxidation of Sulfide to Sulfoxide 98 Using Oxone

To a solution of sulfide (1 mmol) in 1:1 mixture of t-BuOH and water (5 ml) at 0° C. was added Oxone (1.2 mmol) and the RM was warmed to RT and stirred for 4 h. After completion of the reaction, an aqueous solution of sodium bisulphate was added to RM and stirring continued until it was clear. The RM was extracted with ethyl acetate and the organic layers were washed with brine, and dried over sodium sulfate. The solvents were removed under vacuum to afford the crude residue obtained was purified as a mixture of isomers on a silica gel column.

### General Procedure for the Synthesis of Sulfoxide 103 Using Oxone

To a solution of sulfide (1 mmol) in a mixture of (THF: H₂O) (5 ml), was added Oxone (1.2 mmol) at 0° C. and stirred at RT for 1 h. Sodium bisulphate solution was added and stirred until it becomes a clear solution. The RM was extracted with ethyl acetate and organic layers were combined and washed with brine, dried over sodium sulfate. The solvent was removed under vacuum to afford the crude product, which was purified using n-hexane/ethyl acetate mixtures on silica gel column.

Compound 103: HRMS (m/z): 290 (M+Na); ¹H NMR (400 MHz, CDCl₃): δ 2.73 (t, 1H), 3.42-3.61 (m, 3H), 5.0-5.04 (dd, 1H), 5.26 (d, 2H), 6.91 (s, 1H), 7.33-7.41 (m, 5H); ¹³CNMR (100 MHz, CDCl₃): δ 45.10, 47.14, 50.06, 68.66, 128.68, 128.89, 129.11, 134.24, 162.32, 169.34.

### General Procedure for Sulfide Oxidation to Sulfone 105 Using H₂O/ACOH

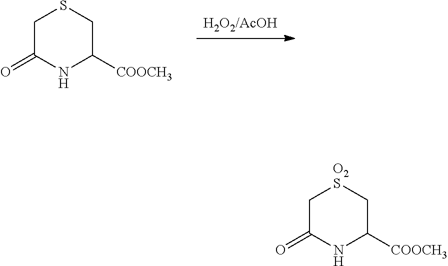

To a solution of the thiomorpholine ester (1 mmol) in glacial acetic acid (11 mmol) at 0° C. was added 30% H₂O₂ (13 mmol) in drop wise. The RM was stirred at 0° C. for 3 h and continued stirring at RT for an additional 48 h. The RM was poured into crushed ice and extracted with ethyl acetate (3×10 ml). The organic layers were washed with saturated sodium bicarbonate solution, brine and then dried over sodium sulfate. The solvent was removed under vacuum to afford the crude, which was purified on silica gel column.

Compound 105: HRMS (m/z): 230 (M+Na); ¹H NMR (300 MHz, CDCl₃): δ 3.41-3.49 (q, 1H), 3.66-3.70 (dd, 1H), 3.89 (s, 3H), 4.01 (d, 2H), 4.59-4.63 (dd, 1H), 7.11 (s, 1H); ¹³CNMR (75 MHz, CDCl₃): δ 49.79, 50.78, 53.95, 56.26, 162.03, 167.67.

### General Procedure for Oxidation of Sulfide Analogs to Sulfone Analog 115 Using Oxone

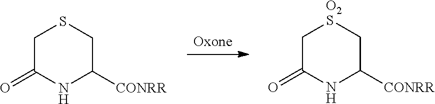

To a solution of sulfide (1 mmol) in 1:1 mixture of t-BuOH and water (5 ml) at 0° C. was added Oxone (10 mmol) and the RM was warmed to RT and stirred for 24 h. After completion of the reaction, an aqueous solution of sodium bisulphate was added to RM and stirring continued until it was clear. The RM was extracted with ethyl acetate and the organic layers washed with brine, and dried over sodium sulfate. The solvents were removed under vacuum to afford the crude residue obtained was purified on a silica gel column.

Compound 115: HRMS (m/z): 305 (M+Na); ¹H NMR (400 MHz, DMSO-d₆): δ 3.52-3.58 (m, 1H), 3.68-3.73 (m, 1H), 4.00-4.05 (dd, 1H), 4.27-4.30 (m, 4H), 7.20-7.31 (m, 5H), 8.44 (s, 1H), 8.71-8.74 (t, 1H); ¹³CNMR (100 MHz, DMSO-d₆): δ 43.02, 49.20, 52.20, 56.14, 127.38, 127.78, 128.73, 139.02, 163.20, 168.03.

### General Procedure for Oxidation of Sulfide Analogs to Sulfoxide Analogs 99, 100& 102 Using Oxone

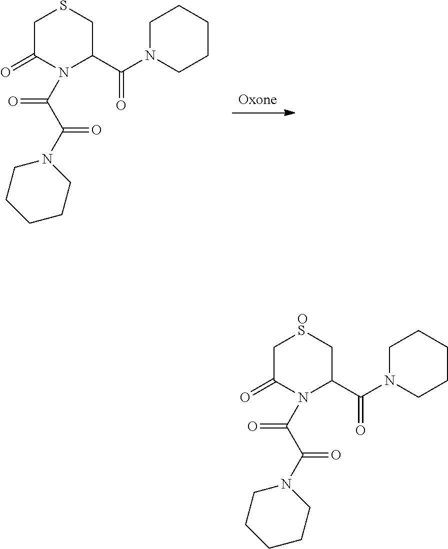

To a solution of the compound (1 mmol) in a mixture of (THF: H₂O) (5 ml), was added Oxone (1.2 mmol) at 0° C. and stirred at RT for 1 h. Sodium bisulphate solution was added and stirred until it becomes a clear solution. Then the RM was extracted with ethyl acetate. Organic layers were combined and washed with brine, dried over sodium sulfate. The solvent was removed under vacuum to afford the crude, which was purified using n-hexane/ethyl acetate mixtures on silica gel column.

Compound 102: HRMS (m/z): 382 (M+Na); ¹H NMR (400 MHz, CDCl₃): δ 1.08-1.33 (m, 12H), 3.09-3.51 (m, 10H), 3.74 (d, 1H), 4.21 (d, 1H), 5.68 (br s, 1H); ¹³CNMR (100 MHz, CDCl₃): δ 11.66, 12.61, 13.03, 14.26, 29.67, 38.53, 41.62, 42.01, 42.68, 50.22, 55.44, 162.73, 164.96, 166.45, 167.14.

### General Procedure for Condensation of Sulfoxide Analogs to the Corresponding Condensation Products 101, 104

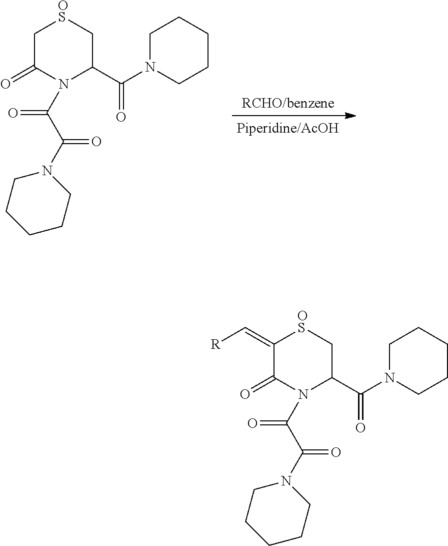

To a solution of sulfoxide amide (1 mmol) in dry benzene (5 ml), piperidine (80 mmol) and acetic acid (80 mmol) were added followed by aldehyde (1.5 mmol). The RM was refluxed at 100° C. using a Dean-Stark set up for 1-1^{1/2} h. After completion of the reaction the solvents were removed and ethyl acetate (20 ml) was added and the organic layer was washed with 3N HCl, saturated sodium bicarbonate solution, brine and dried over sodium sulfate. The solvent was removed under vacuum to afford the crude aldol product and was purified on a silica gel column.

Compound 101: HRMS (m/z): 494 (M+Na); ¹H NMR (400 MHz, CDCl₃): δ 1.57-2.00 (m, 12H), 3.14-3.74 (m, 10H), 5.72 (br s, 1H), 7.34-7.44 (t, 3H), 7.63 (d, 2H), 8.38 (br s, 1H); ¹³CNMR (100 MHz, CDCl₃): δ 24.39, 24.51, 24.75, 25.11, 25.48, 26.01, 26.33, 29.65, 42.00, 44.32, 46.68, 46.93, 47.35, 47.71, 50.00, 129.06, 131.11, 131.37, 131.99, 132.03, 152.07, 161.34, 161.81, 166.50, 166.75.

Compound 104: HRMS (m/z): 537 (M+Na); ¹H NMR (400 MHz, CDCl₃): δ 1.5-1.7 (m, 15H), 3.08-3.80 (m, 13H), 5.67 (br d, 1H), 6.65 (d, 2H), 7.68 (d, 2H), 8.24 (s, 1H); ¹³CNMR (100 MHz, CDCl₃): δ 24.57, 24.67, 24.81, 25.07, 25.49, 25.92, 26.32, 40.02, 41.89, 44.28, 46.62, 47.15, 47.41, 111.65, 119.65, 135.47, 153.26, 161.41, 162.14, 166.89, 167.08.

### General Procedure for Condensation of Sulfone Esters 106, 108, 110, 112, 113, 118, 120 (from 105) with Aldehydes

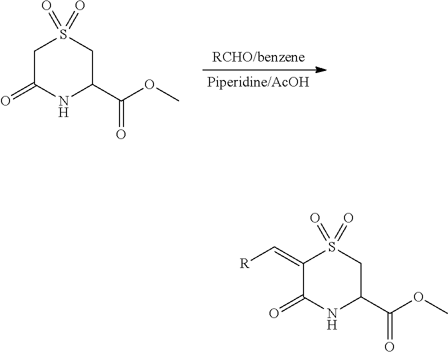

To a solution of sulfone ester (1 mmol) in dry benzene (5 ml), piperidine (80 mmol) and acetic acid (80 mmol) were added followed by aldehyde (1.5 mmol). The RM was refluxed at 100° C. using a Dean-Stark set up for 1-1^{1/2} h. After completion of the reaction the solvents were removed and ethyl acetate (20 ml) was added and the organic layer was washed with 3N HCl, saturated sodium bicarbonate solution, brine and dried over sodium sulfate. The solvent was removed under vacuum to afford the crude aldol product and was purified on a silica gel column.

Compound 106: HRMS (m/z): 318 (M+Na); ¹H NMR for two isomers (300 MHz, CDCl₃): δ 3.44-3.84 (m, 2H), 3.89 (s, 3H), 4.64-4.73 (m, 1H), 6.84 & 7.04 (br s, 1H), 7.4 (s, 1H), 7.42-7.51 (m, 2H), 7.82-7.93 (m, 2H).

Compound 108: HRMS (m/z): 348 (M+Na); ¹H NMR for two isomers (400 MHz, DMSO-d₆): δ 3.68 (s, 3H), 3.74 (s, 3H), 3.80-4.03 (m, 2H), 4.62-4.69 (m, 1H), 6.98-7.02 (t, 2H), 7.86-7.93 (dd, 2H), 7.55 & 8.18 (s, 1H), 8.72 & 8.87 (d, 1H); ¹³CNMR (100 MHz, DMSO-d₆): δ 22.09, 22.68, 44.20, 49.40, 50.07, 52.55, 52.67, 53.23, 53.36, 55.97, 56.02, 114.39, 114.51, 124.30, 129.36, 130.04, 134.99, 135.43, 142.48, 147.00, 161.01, 162.62, 162.77, 169.83, 170.18.

Compound 110: HRMS (m/z): 332 (M+Na); ¹H NMR for two isomers (400 MHz, DMSO-d₆): δ 2.32-2.33 (d, 3H), 3.66 (s, 3H), 3.83-4.05 (m, 2H), 4.63-4.72 (dd, 1H), 7.23-7.33 (m, 2H), 7.72-7.76 (m, 2H), 7.58 & 8.22 (s, 1H), 8.80 & 8.93 (d, 1H); ¹³CNMR (100 MHz, DMSO-d₆): δ 21.62, 49.47, 50.00, 52.38, 52.74, 53.24, 53.40, 128.56, 128.79, 129.28, 129.39, 129.58, 131.53, 132.22, 132.32.

Compound 112: HRMS (m/z): 352 (M+Na); ¹H NMR for two isomers (400 MHz, CDCl₃+DMSO-d₆): δ 3.70 (m, 2H), 3.79-3.89 (m, 2H), 4.62-4.65 (m, 1H), 7.39-7.43 (m, 2H), 7.76-7.88 (dd, 2H), 7.70 & 8.32 (s, 1H), 8.71 & 8.72 (d, 1H); ¹³CNMR (100 MHz, CDCl₃+DMSO-d₆): δ 54.31, 52.38, 54.93, 56.70, 57.27, 57.97, 58.05, 132.28, 133.44, 134.49, 135.16, 137.13, 137.81, 138.09, 138.29, 142.20, 142.31, 147.28, 151.49, 165.03, 166.05, 173.60, 173.98.

Compound 113: HRMS (m/z): 363 (M+Na); ¹H NMR for two isomers (400 MHz, DMSO-d₆): δ 3.69 (s, 3H), 3.99-4.10 (m, 2H), 4.68-4.69 (dd, 1H), 7.92-7.95 (m, 2H), 8.24-8.27 (m, 2H), 7.86 & 8.39 (s, 1H), 8.99 & 9.02 (m, 1H); ¹³CNMR (100 MHz, DMSO-d₆): δ 49.64, 50.04, 51.85, 53.30, 53.52, 123.53, 123.63, 128.79, 132.29, 132.39, 136.20, 136.69, 138.54, 138.96, 141.06, 144.93, 148.73, 159.84, 161.00, 169.69, 169.93.

Compound 118: HRMS (m/z): 364 (M+Na); ¹H NMR (400 MHz, CDCl₃): δ 0.76-0.82 (t, 3H), 0.98-1.01 (t, 3H), 1.60 (s, 3H), 2.12-2.4 (m, 3H), 2.5-2.6 (m, 1H), 3.42-3.87 (m, 5H), 4.42-4.44 (t, 1H), 4.58 (t, 1H), 5.22 (s, 1H), 5.40-5.62 (m, 1H), 6.0-6.28 (m, 1H), 7.27 (br s, 1H); ¹³CNMR (100 MHz, CDCl₃): δ 12.59, 20.50, 20.67, 25.35, 25.46, 25.52, 34.40, 34.52, 34.83, 34.93, 47.97, 48.53, 48.57, 48.63, 48.96, 50.15, 50.25, 50.44, 53.88, 54.16, 54.30, 68.55, 68.69, 69.01, 69.07, 113.25, 113.40, 114.93, 115.48, 121.04, 121.22, 121.25, 144.27, 145.13, 145.46, 146.20, 147.72, 147.82, 147.95, 165.02, 167.82, 167.97.

Compound 120: ¹H NMR (400 MHz, CDCl₃): δ 3.43-3.64 (m, 4H), 3.85-4.06 (m, 4H), 4.51-4.57 (m, 1H), 4.56 (t, 1H), 6.63-6.67 (d, 1H), 7.25-7.28 (t, 3H), 7.71-7.75 (t, 1H), 8.51-8.64 (dd, 2H).

### General Procedure for Condensation of Sulfone Amides 116 (from 15) with Aldehydes

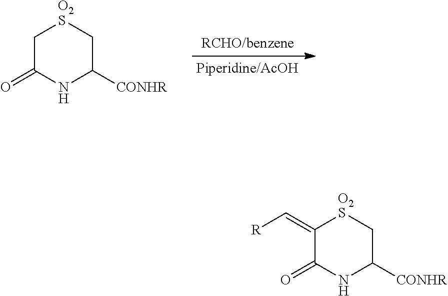

To a solution of sulfone amide (1 mmol) in dry benzene (5 ml), piperidine (80 mmol) and acetic acid (80 mmol) were added followed by aldehyde (1.5 mmol). The RM was refluxed at 100° C. using a Dean-Stark set up for 1-1^{1/2} h. After completion of the reaction the solvents were removed and ethyl acetate (20 ml) was added and the organic layer was washed with 3N HCl, saturated sodium bicarbonate solution, brine and dried over sodium sulfate. The solvent was removed under vacuum to afford the crude aldol product and was purified on a silica gel column.

Compound 116: HRMS (m/z): 393 (M+Na); ¹H NMR for two isomers (400 MHz, DMSO-d₆): δ 3.78-3.84 (m, 1H), 3.95-4.00 (m, 1H), 4.21-4.50 (m, 3H), 7.24-7.50 (m, 8H), 7.83-7.91 (dd, 2H), 8.67-8.72 (dd, 2H), 8.29 & 7.68 (s, 1H); ¹³CNMR (100 MHz, DMSO-d₆): δ 43.14, 50.64, 51.15, 52.23, 52.75, 127.36, 127.76, 127.88, 128.69, 128.79, 131.57, 131.96, 132.22, 132.77, 133.25, 138.95, 139.04, 142.39, 146.97, 160.48, 161.81, 167.53, 168.13.

### General Procedure for Hydrogenation to Obtain 107, 109, 111, 114, 117, 119, 122

To a solution of aldol product (0.5 mmol) in methanol (5 ml), was added catalytic amount of 10% Pd—C. The RM was stirred under hydrogen atmosphere using a balloon containing hydrogen at RT for 1-2 hr. The RM was filtered through a silica gel pad using DCM and followed by methanol to obtain the crude, which was further purified on a silica gel column.

Compound 107: HRMS (m/z): 320 (M+Na); ¹H NMR (400 MHz, CDCl₃): δ 3.31-3.46 (m, 2H), 3.51-3.81 (m, 2H), 3.77 (s, 3H), 3.81-4.03 (t, 1H), 4.44-4.47 (m, 1H), 6.83-6.88 (d, 1H), 7.21-7.35 (m, 5H); ¹³CNMR (100 MHz, CDCl₃): δ 30.47, 31.59, 49.10, 49.81, 49.87, 50.31, 53.80, 67.03, 67.08, 127.33, 127.53, 128.67, 128.73, 129.58, 129.92, 135.48, 136.49, 164.75, 165.33, 167.44, 168.00.

Compound 109: HRMS (m/z): 350 (M+Na); ¹H NMR (400 MHz, CDCl₃): δ 3.21-3.46 (m, 4H), 3.75 (s, 3H), 3.81 (s, 3H), 3.96-3.99 (t, 1H), 4.33-4.46 (m, 1H), 6.79-6.86 (m, 2H), 7.21-7.26 (m, 2H); ¹³CNMR (100 MHz, CDCl₃): δ 30.19, 31.31, 48.95, 49.56, 49.94, 50.31, 53.80, 55.22, 55.27, 67.15, 67.29, 114.03, 114.09, 127.02, 128.04, 129.26, 130.79, 131.12, 158.81, 158.96, 164.83, 165.43, 167.43, 168.06.

Compound 111: HRMS (m/z): 334 (M+Na); ¹H NMR (400 MHz, DMSO-d₆): δ 2.3 (s, 3H), 3.66 (s, 3H), 3.21-3.29 (m, 2H), 3.62-3.67 (m, 3H), 4.37-4.45 (m, 1H), 4.54-4.64 (m, 1H), 7.05-7.15 (m, 4H), 8.52 (s, 1H); ¹³CNMR (100 MHz, DMSO-d₆): δ 20.58, 27.09, 28.36, 47.88, 48.87, 49.41, 49.64, 52.84, 65.32, 65.36, 128.72, 128.78, 129.83, 128.97, 134.46, 135.06, 135.34, 135.52, 165.06, 165.26, 169.22.

Compound 114: HRMS (m/z): 335 (M+Na); ¹H NMR (400 MHz, CD₃OD): δ 3.22-3.29 (m, 4H), 3.66-3.79 (m, 3H), 4.35 (t, 1H), 4.59-4.61 (m, 1H), 6.63-6.65 (d, 2H), 7.03-7.06 (d, 2H); ¹³CNMR (100 MHz, CD₃OD): δ 29.23, 29.30, 30.83, 30.91, 48.55, 50.03, 50.11, 50.80, 52.29, 52.33, 66.55, 66.92, 115.25, 115.35, 125.10, 126.02, 129.98, 130.19, 146.08, 146.42, 163.79, 166.41, 166.59, 168.66, 169.07, 169.24.

Compound 117: HRMS (m/z): 395 (M+Na); ¹H NMR (400 MHz, DMSO-d₆): δ 3.07-3.16 (m, 4H), 3.75-3.82 (m, 1H), 4.27-4.32 (m, 2H), 4.57-4.60 (t, 1H), 7.16-7.29 (m, 10H), 8.23& 8.45 (s, 1H), 8.73-8.78 (d, 1H); ¹³CNMR (100 MHz, DMSO-d₆): δ 27.05, 29.11, 43.04, 43.12, 49.88, 50.30, 51.34, 51.45, 65.16, 65.96, 126.81, 126.97, 127.39, 127.76, 128.67, 128.72, 128.75, 129.41, 129.64, 138.07, 138.85, 139.00, 165.26, 165.83, 167.25, 168.25.

Compound 119: HRMS (m/z): 368 (M+Na); ¹H NMR (400 MHz, CDCl₃): δ 0.50 (s, 3H), 0.77-0.86 (dd, 6H), 1.16-1.81 (m, 1H), 3.42-3.45 (m, 1H), 3.63-3.74 (m, 2H), 3.87 (s, 3H), 4.56 (t, 1H), 6.80-6.82 (d, 1H); ¹³CNMR (100 MHz, CDCl₃): δ 13.81, 14.46, 24.32, 25.11, 25.29, 25.61, 27.79, 27.84, 27.88, 28.69, 28.76, 28.88, 28.98, 30.05, 42.41, 45.14, 49.61, 49.93, 50.04, 50.12, 50.33, 50.54, 50.60, 50.69, 53.87, 65.40, 65.49, 66.12, 66.24, 165.38, 165.70, 167.75, 167.94.

Compound 122: HRMS (m/z): 354 (M+Na); ¹H NMR (400 MHz, CDCl₃+DMSO-d₆): δ 3.28-3.70 (m, 4H), 3.78 (s, 3H), 4.17-4.39 (br s, 1H), 4.59-4.61 (m, 1H), 7.21-7.34 (m, 5H), 8.17 & 8.30 (br s, 1H); ¹³CNMR (100 MHz, CDCl₃+DMSO-d₆): δ 33.98, 34.31, 34.93, 53.65, 54.55, 54.79, 54.85, 58.03, 71.43, 131.69, 131.81, 133.23, 134.30, 141.74, 169.92, 170.06, 173.29, 173.58.

### General Procedure for Sulfide Oxidation to Sulfone 121 Using Oxone

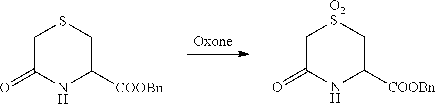

To a solution of sulfide ester (1 mmol) in a 1:1 mixture of (THF: H₂O) (5 ml), was added Oxone (10 mmol) at 0° C. and RM stirred at RT for 1 h. Sodium bisulphate solution was added to the RM and stirred until it becomes clear solution. The RM was extracted with ethyl acetate and the organic layers were combined and washed with brine, dried over sodium sulfate. The solvents were removed under vacuum to afford the crude product, which was purified using n-hexane/ethyl acetate mixtures on a silica gel column.

Compound 121: HRMS (m/z): 306 (M+Na); ¹H NMR (400 MHz, CDCl₃): δ 3.27-3.59 (m, 5H), 3.91 (s, 1H), 4.50 (m, 1H), 5.16 (s, 2H), 7.29 (m, 5H); ¹³CNMR (100 MHz, CDCl₃): δ 49.26, 50.86, 56.09, 68.76, 128.64, 128.75, 128.96, 134.20, 162.77, 167.60.

### General Procedure for Condensation of Sulfone Esters 123-128 and 135 (from 121) with Aldehydes

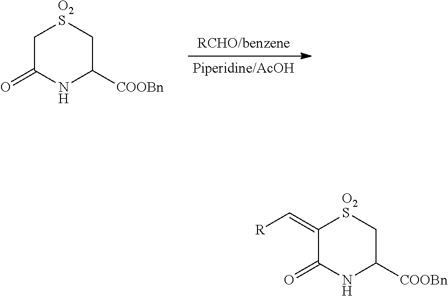

To a solution of sulfone ester (1 mmol) in dry benzene (5 ml), piperidine (80 mmol) and acetic acid (80 mmol) were added followed by aldehyde (1.5 mmol). The RM was refluxed at 100° C. using a Dean-Stark set up for 1-1^{1/2} h. After completion of the reaction the solvents were removed and ethyl acetate (20 ml) was added and the organic layer was washed with 3N HCl, saturated sodium bicarbonate solution, brine and dried over sodium sulfate. The solvent was removed under vacuum to afford the crude aldol product and was purified on a silica gel column.

Compound 123: HRMS (m/z): 408 (M+Na); ¹H NMR (400 MHz, CDCl₃): δ 2.41 (t, 3H), 3.40-3.57 (m, 1H), 3.73-3.80 (dd, 1H), 4.73 (m, 1H), 5.26 (m, 2H), 6.73 (s, 1H), 7.21-7.41 (m, 8H), 7.74-7.89 (m, 2H), 8.45 (s, 1H).

Compound 124: HRMS (m/z): 428 (M+Na); ¹H NMR (400 MHz, CDCl₃): δ 3.42-3.58 (m, 1H), 3.74-3.82 (dd, 1H), 4.63-4.73 (m, 1H), 5.22-5.32 (m, 2H), 6.76 (s, 1H), 7.26-7.43 (m, 8H), 7.75 (d, 1H, 8.41 (s, 1H).

Compound 125: HRMS (m/z): 424 (M+Na); ¹H NMR (400 MHz, CDCl₃): δ 3.41-3.55 (m, 1H), 3.74-3.78 (d, 1H), 3.87 (s, 3H), 4.73 (m, 1H), 5.28 (t, 2H), 6.69 (s, 1H), 6.91-6.97 (m, 2H), 7.24-7.41 (m, 6H), 7.87 (t, 1H), 8.02 (d, 1H), 8.39 (s, 1H).

Compound 126: HRMS (m/z): 439 (M+Na); ¹H NMR (400 MHz, CDCl₃+DMSO): δ 3.75 (t, 1H), 4.65 (t, 1H), 5.16-5.26 (m, 2H), 7.34-7.46 (m, 6H), 7.85 (s, 1H), 7.90 (d, 1H), 8.19 (d, 1H), 8.25 (d, 1H), 8.46 (s, 1H).

Compound 127: HRMS (m/z): 437 (M+Na); ¹H NMR (400 MHz, CDCl₃+DMSO): δ 3.29 (s, 6H), 3.63 (t, 1H), 3.92 (d, 1H), 4.89 (d, 1H), 5.47 (d, 2H), 6.76 (s, 1H), 6.87 (d, 2H), 7.46 (s, 1H), 7.57 (m, 4H), 8.06 (d, 2H), 8.49 (s, 1H); ¹³CNMR (100 MHz CDCl₃+DMSO): δ 40.02, 49.31, 53.62, 68.97, 111.12, 118.51, 121.63, 128.80, 128.93, 129.20, 134.05, 136.75, 150.49, 153.50, 161.41, 167.45.

Compound 128: HRMS (m/z): 394 (M+Na); ¹H NMR (400 MHz, CDCl₃): δ 3.40-3.59 (m, 1H), 3.74-3.82 (m, 1H), 4.72-4.85 (m, 1H), 5.25-5.29 (m, 2H), 7.0 (s, 1H), 7.26-7.51 (m, 8H), 7.8 (d, 1H), 7.84 (d, 1H), 8.50 (s, 1H).

Compound 135: HRMS (m/z): 440 (M+Na); ¹H NMR (400 MHz, CDCl₃): δ 0.73-0.82 (m, 3H), 0.98 (t, 3H), 1.59 (s, 3H), 2.06-2.32 (m, 2H), 2.49-2.53 (t, 2H), 3.43-3.71 (m, 2H), 4.36 (m, 1H), 4.55 (br s, 1H), 6.0 (m, 1H), 5.24 (d, 2H), 5.56 (m, 2H), 6.96 (d, 1H), 7.25 (m, 5H).

### General Procedure for Hydrogenation to Obtain the Acids 129-134

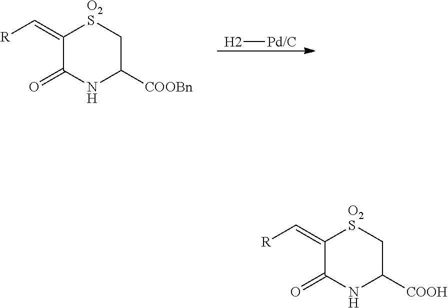

To a solution of aldol product (0.5 mmol) in methanol (5 ml), was added catalytic amount of 10% Pd—C. The RM was stirred under hydrogen atmosphere using a balloon containing hydrogen at RT for 1-2 hr. The RM was filtered through a silica gel pad using DCM and followed by methanol to obtain the crude, which was further purified on a silica gel column.

Compound 129: HRMS (m/z): 320 (M+Na); ¹H NMR (400 MHz, CDCl₃): δ 2.77 (d, 3H), 2.95-3.6 (m, 5H), 4.15-4.34 (m, 1H), 7.06 (d, 2H), 7.21 (t, 2H).

Compound 130: HRMS (m/z): 340 (M+Na); ¹H NMR (400 MHz, CD₃OD): δ 3.05-3.78 (m, 5H), 4.21-4.44 (m, 2H), 7.17-7.35 (m, 5H).

Compound 131: HRMS (m/z): 336 (M+Na); ¹H NMR (400 MHz, CD₃OD): δ 3.23-3.40 (m, 4H), 3.56-3.77 (m, 4H), 4.25-4.37 (m, 1H), 6.84 (d, 2H), 7.25 (d, 2H).

Compound 132: HRMS (m/z): 327 (M+H); ¹H NMR (400 MHz, CD₃OD): δ 2.87 (d, 6H), 3.23-3.36 (m, 4H), 3.54-3.61 (m, 1H), 4.28-4.32 (dd, 1H), 6.70 (t, 2H), 7.17 (t, 2H).

Compound 133: HRMS (m/z): 283 (M+); ¹H NMR (400 MHz, CDCl₃+CD3OD): δ 2.91-3.33 (m, 3H), 3.82-4.0 (m, 1H), 6.80-7.02 (m, 4H).

Compound 134: HRMS (m/z): 352 (M+Na); ¹H NMR (400 MHz, CDCl₃): δ 0.53 (s, 4H), 0.80-0.92 (m, 12H), 3.30-4.04 (m, 6H), 4.31 (m, 1H), 4.52 (m, 1H).

### General Procedure for Sulfide Oxidation to Sulfones 136 & 137 Using Oxone

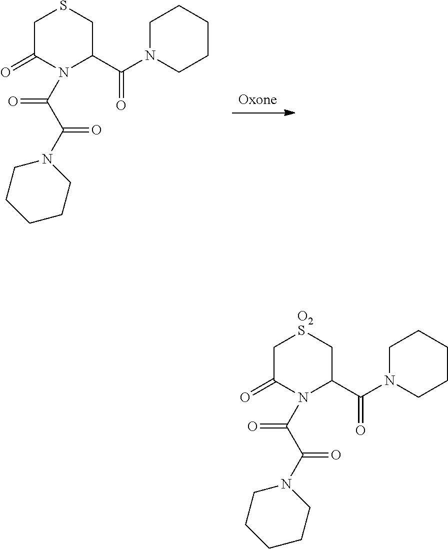

To a solution of the compound (1 mmol) in a 1:1 mixture of (THF: H₂O) (5 ml), was added Oxone (10 mmol) at 0° C. and RM stirred at RT for 1 h. Sodium bisulphate solution was added to the RM and stirred until it becomes clear solution. The RM was extracted with ethyl acetate and the organic layers were combined and washed with brine, dried over sodium sulfate. The solvents were removed under vacuum to afford the crude product, which was purified using n-hexane/ethyl acetate mixtures on a silica gel column.

Compound 136: MS (m/z): 399 (M+); ¹H NMR (400 MHz, CDCl₃): δ 1.57-1.63 (m, 12H), 3.2 (br s, 2H), 3.45-3.59 (m, 7H), 3.90 (m, 1H), 4.18 (d, 1H), 4.37 (d, 1H), 5.82 (t, 1H); ¹³CNMR (100 MHz, CDCl₃): δ 24.26, 24.35, 24.68, 25.02, 25.21, 26.18, 42.07, 44.54, 46.85, 49.25, 51.20, 58.58, 128.31, 161.57, 163.98, 164.41, 166.05.

Compound 137: MS (m/z): 375 (M+); ¹H NMR (400 MHz, CDCl₃): δ 1.07-1.30 (m, 12H), 3.10-3.51 (m, 9H), 3.85-3.89 (m, 1H), 4.16 (d, 1H), 4.40 (br d, 1H), 5.70 (t, 1H); ¹³CNMR (100 MHz, CDCl₃): δ 11.63, 12.51, 12.97, 14.07, 38.42, 41.42, 41.89, 42.23, 49.55, 51.07, 58.41, 162.54, 163.90, 165.65, 166.20.

### General Procedure for the Reduction of Thiomorpholine Ester 138 with LiBH₄

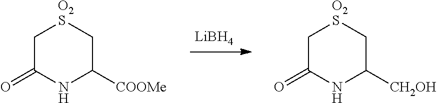

To a solution of ester (1 mmol) in dry THF (2 ml), was added LiBH4 in THF (1.2 mmol) drop wise under inert atmosphere at −70° C. After stirring for 1 h at −70° C., methanol was added to the RM and stirring continued for additional 2-3 h. The solvent was removed under vacuum and the crude was purified using chloroform/methanol mixtures on a silica gel column.

Compound 138: HRMS (m/z): 170 (M+Na); ¹H NMR (400 MHz, CD₃OD): δ 2.73-2.77 (m, 1H), 2.90 (m, 1H), 3.19-3.31 (m, 2H), 3.52-3.68 (m, 3H); ¹³CNMR (100 MHz, CD₃OD): δ 26.08, 29.17, 55.94, 63.02, 169.08.

### General Procedure for the MOM Analog 139 Formation

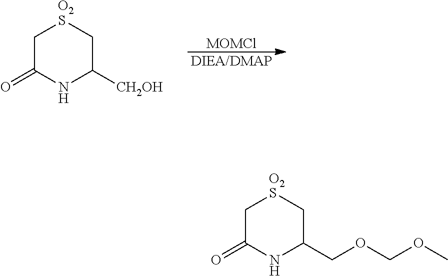

To a solution of alcohol (1 mmol) in dry THF (5 ml), were added DIEA (4 mmol) and DMAP and the RM was cooled to 0° C. MOM chloride (3 mmol) was added drop by drop and the RM stirred at RT for 4 h. The RM was washed with water and the organic layers were collected and dried over sodium sulfate. The solvent was removed under vacuum to afford the crude, which was purified using n-hexane/ethyl acetate mixtures on a silica gel column.

Compound 139: HRMS (m/z): 214 (M+Na); ¹H NMR (400 MHz, CDCl₃): δ 2.57-2.63 (q, 1H), 2.63-2.79 (dd, 1H), 3.21 (s, 2H), 3.30 (s, 3H), 3.59-3.63 (q, 1H), 3.80 (q, 1H), 3.78-3.82 (br s, 1H), 4.55 (s, 2H), 6.98 (br s, 1H); ¹³CNMR (100 MHz, CDCl₃): δ 26.99, 30.02, 54.42, 55.51, 69.52, 96.65, 167.48.

Other compounds of the present invention whose synthesis was not explicitly disclosed above may be prepared readily according to the following reaction Schemes (in which variables are as defined before or are defined) using readily available starting materials, reagents and conventional synthesis procedures. In these reactions, it is also possible to make use of variants, which are themselves known to those of ordinary skill in this art, but are not mentioned in greater detail. It should be noted that the schemes presented below are prophetic.

It will be appreciated that those the synthesis of the compounds of the present invention may be carried out using reagents and reactions known to the skilled artisan. The specific synthetic procedures above may also act as a guide for synthesis of related compounds of the present invention.

It should be understood, of course, that the foregoing relates to exemplary embodiments of the invention and that modifications may be made without departing from the spirit and scope of the invention as set forth in the following claims.

We claim:

1. A compound (I) comprising the formula: 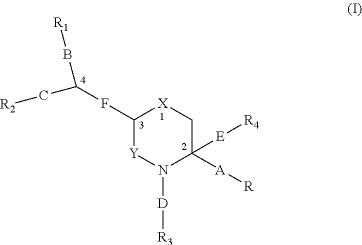
wherein:
X is —S—, —S(O)—, or —S(O)₂—;
Y is —CH₂—, 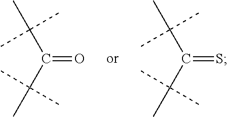
A is —(CH₂)ₘ— where m=0, 1, 2, 3, or 4, —(CH═CR₅)ₙ— where R₅ is a hydrogen, an alkyl, a cycloalkyl, a heterocyclyl, an aryl or an heteroaryl and n=0, 1, or 2, -alkenylene-, -alkynylene-, -cycloalkylene-, -heterocyclylene-, -arylene-, -fused heterocyclylarylene-, -fused heterocyclylheterocyclylene-, -fused heterocyclylheteroarylene-, -fused heterocyclylcycloalkylene-, -fused heteroarylarylene-, -fused heteroarylheterocyclylene-, -fused heteroarylheteroarylene-, or -fused heteroarylcycloalkylene-;
R is —C(O)R₆, —OR₇, —NR₈R₉, —SR₁₀, —S(O)R₁₁, —S(O)₂R₁₂, —S(O)₂NHC(O)-alkyl, —S(O)₂NHC(O)-aryl, —S(O)₂NHC(O)-heteroaryl, —S(O)₂NHC(O)-alkylenearyl, —S(O)₂NHC(O)-alkyleneheteroryl, —S(O)₂NHC(O)-arylenealkyl, —CHR₁₃R₁₄, —CN, -J, -alkylene-J, -arylene-J, -cycloalkylene-J, -alkyleneheterocyclylene-J, —alkenylheterocyclylene-J, -alkynyleneheterocyclylene-J, -alkyleneheteroarylene-J, -alkenylheteroarylene-J, —NHCH2-J, —NR₁₃CHR₁₄-J, —NHS(O)₂-alkyl, —NHS(O)₂-aryl, —NHS(O)₂-heteroaryl, —NHS(O)₂-cycloalkyl, —NHS(O)₂-fused heteroaryl, —NHS(O)₂-alkylene-J, —NHS(O)₂-arylene-J, —NHS(O)₂-heteroarylene-J, —NHS(O)₂-cycloalkylene-J, —NHS(O)₂-fused heteroarylene-J, —P(O)(OH)(O-alkyl), or —P(O)(O-alkyl)₂;
wherein J is —H; —OH—; —COOH; —P(O)(OH)₂; —S(O)₂OH; —B(OH)₂; -acid isostere; 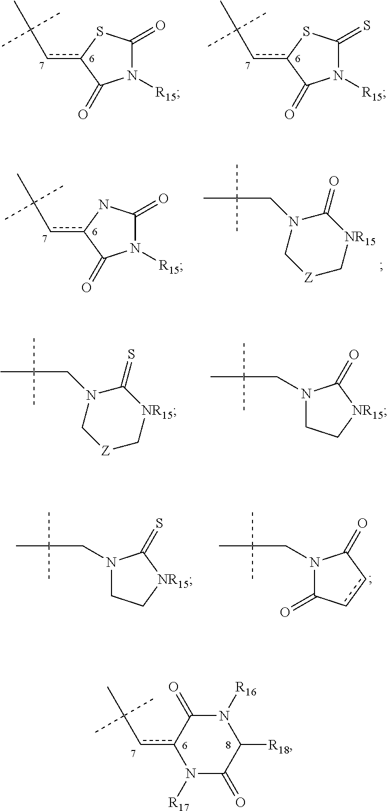
wherein Z is —CR₁₃R₁₄—; —O—; —NR₁₅—; —S—; —S(O)—; or —S(O)₂—; wherein the stereocenters 6 & 7 may possess an E, Z or EZ configuration and stereocenter 8 may possess an R, S or RS configuration;
wherein R₁₅, R₁₆, and R₁₇ are each independently —H; -alkyl; -cycloalkyl; -aryl; -alkylenearyl; -alkenylenearyl; -alkynylenearyl; -heterocyclyl; -heteroaryl; -alkyleneheterocyclyl; -alkenyleneheterocyclyl; -alkynyleneheterocyclyl; -alkyleneheteroaryl; -alkenyleneheteroaryl; or -alkynyleneheteroaryl and R₁₈ is —H; -alkyl; -cycloalkyl; -aryl; -heterocyclyl; -heteroaryl; -alkylenecycloalkyl; -alkylenearyl; -alkyleneheterocyclyl; or -alkyleneheteroaryl;
wherein R₆ is —H; —OR₁₉; —CHR₂₀R₂₁; —NR₂₂R₂₃; —NHS(O)₂-alkyl; —NHS(O)₂-aryl; —NHS(O)₂-heteroaryl; —NHS(O)₂-heterocyclyl; —NHS(O)₂-alkylenearyl; —NHS(O)₂-alkyleneheteroaryl; —NHS(O)₂-alkyleneheterocyclyl; —NHS(O)₂-arylenealkyl; -cycloalkyl: —heterocyclyl; -aryl; -aryloxyalkyl; -cycloalkylaryl; -heteroaryl; -alkenyleneheteroaryl; -alkynyleneheteroaryl; -alkenylenearyl; -alkynylenearyl; -fused cycloalkyl; -fused aryl; -fused heteroaryl; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; or -fused heteroarylheterocyclyl;
wherein R₁₉ is —H; -alkyl; -cycloalkyl; -perhaloalkyl; -heterocyclyl; -aryl; -heteroaryl; -alkylene-heteroaryl; -alkylene-aryl; or -arylene-alkyl;
and wherein R₂₀ and R₂₁ are each independently —H; -alkyl; -cycloalkyl; -aryl; -alkylenearyl; -alkenylenearyl; -alkynylenearyl; -heterocyclyl; -alkyleneheterocyclyl; -alkenyleneheterocyclyl; -alkynyleneheterocyclyl; -heteroaryl; -alkyleneheteroaryl; -alkenyleneheteroaryl; or -alkynyleneheteroaryl;
and wherein R₂₂ and R₂₃ are each independently: —H; —C(O)CR₂₉R₃₀NH[C(O)CR₂₉R₃₀NH]nR₃₁; wherein n=0, 1, 2, or 3; —C(O)CH2CR₂₉R₃₀NH[C(O)CHR₂₉NH]nR₃₁; wherein n=0, 1, 2, or 3; —C(O)CR₂₉R₃₀NH[C(O)CH₂CHR₂₉NH]nR₃₁; wherein n=0, 1, 2, or 3; -alkyl-J; -cycloalkyl-J; -aryl-J; -alkylenearyl-J; -alkenylenearyl-J; -alkynylenearyl-J; -heterocyclyl-J; -alkyleneheterocyclyl-J; -alkenyleneheterocyclyl-J; -alkynyleneheterocyclyl-J; -aryloxyalkyl-J; -alkoxyaryl-J; -heteroaryl-J; -alkyleneheteroaryl-J; -alkenyleneheteroaryl-J; -alkynyleneheteroaryl-J; -fused cycloalkyl-J; -fused aryl-J; -fused heteroaryl-J; -fused cycloalkylaryl-J; -fused arylcycloalkyl-J; -fused heterocyclylaryl-J; -fused arylheterocyclyl-J; -fused cycloalkylheteroaryl-J; -fused heteroarylcycloalkyl-J; -fused heterocyclylheteroaryl-J; or -fused heteroarylheterocyclyl-J; and wherein R₂₂ and R₂₃ together may form a ring having the formula —(CH₂)ₐ-M-(CH₂)_{b}— bonded to the nitrogen atom to which R₂₂ and R₂₃ are attached and wherein a and b are independently 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₈)—; —N(R₂₇)S(O)₂—; —S(O)₂N(R₂₇)—; —C(O)—O—; —O—C(O)—; —N(R₂₇)S(O)₂N(R₂₈)—; 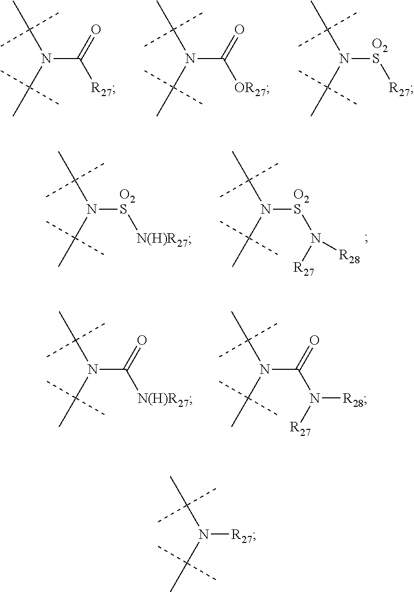
wherein R₂₇ and R₂₈ are each independently —H; —CN, —NO₂; -alkyl; -cycloalkyl; -heterocyclyl; -aryl; -heteroaryl; —C(O)—O-alkyl; —C(O)—O-aryl; —C(O)—O-alkylenearyl; -alkylene-heterocyclyl; -alkylene-cycloalkyl; -alkylene-aryl; or -alkylene-heteroaryl;
wherein R₂₄ is —H; —C(O)R₆; —SR₁₀; —S(O)R₁₁; —S(O)₂R₁₂; —S(O)₂NHC(O)-alkyl; —S(O)₂NHC(O)-aryl; —S(O)₂NHC(O)-heteroaryl; —S(O)₂NHC(O)-alkylenearyl; —S(O)₂NHC(O)-alkyleneheteroryl; —S(O)₂NHC(O)-arylenealkyl; an acid isostere; —CN; —P(O)(OH)(O-alkyl); —P(O)(O-alkyl)₂; —P(O)(OH)₂; —C(O)OH; or -acid isostere;
wherein R₂₅ and R₂₆ are each independently —H; -alkyl; cycloalkyl; -aryl; -alkylenearyl; -alkenylenearyl; -alkynylenearyl; -heterocyclyl; -alkyleneheterocyclyl; -alkenyleneheterocyclyl; or -alkynyleneheterocyclyl; and wherein R₂₅ and R₂₆ together may form a ring having the formula —(CH₂)ₐ-M-(CH₂)_{b}— bonded to the nitrogen atom to which R₂₂ and R₂₃ are attached wherein a and b are independently equal to 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—; 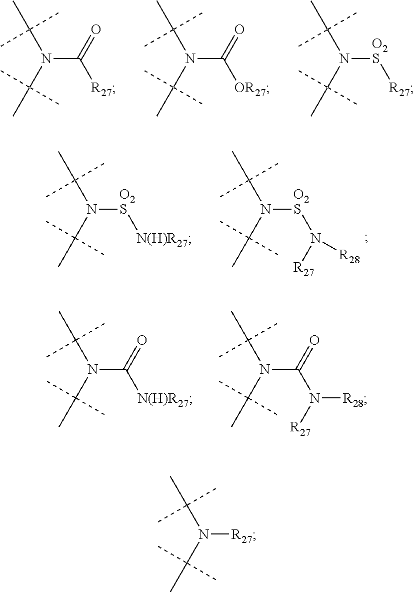
wherein R₂₉, R₃₀ and R₃₁ are each independently —H; -alkyl; -cycloalkyl; -aryl; -heterocyclyl; -heteroaryl; -alkylenecycloalkyl; -alkylenearyl-J; -alkyleneheteroaryl; or -alkylene-J;
wherein R₇ is: —H; -alkyl; -cycloalkyl: -heterocyclyl; -aryl; -aryloxy; -alkoxy; -heteroaryloxy; -heteroaryl; -alkyleneheteroaryl; -alkenyleneheteroaryl; -alkynyleneheteroaryl; -alkylenearyl; -alkenylenearyl; -alkynylenearyl; -perhaloalkyl; -alkylene-T-R₂₄; -cycloalkylene-T-R₂₄; -heterocyclylene-T-R₂₄; -arylene-T-R₂₄; -heteroarylene-T-R₂₄; -alkylene-C(O)NR₂₅R₂₆; -alkylene-NR₂₅R₂₆; -fused cycloalkyl; -fused aryl; -fused heteroaryl; -fused cycloalkylaryl; -fused arylcycloalkyl; -fused heterocyclylaryl; -fused arylheterocyclyl; -fused heteroarylcycloalkyl; -fused heterocyclylheteroaryl; or -fused heteroarylheterocyclyl; wherein T is alkylene; arylene; heteroarylene; —(CH₂)_{d}—, d=0 or 1; —O—; —N(R₂₇)—; —S—; —S(O)—; —S(O)₂—; —O—S(O)—; and —O—C(O)—; —C(O)—O—; —N(R₂₇)C(O)—; —C(O)N(R₂₇)—; —N(R₂₇)C(O)N(R₂₈)—; —N(R₂₇)S(O)₂—; —S(O)₂N(R₂₇)—; —N(R₂₇)S(O)₂N(R₂₈)—; —C(O)N(R₂₇)S(O)₂—; —N(R₂₇)C(O)—O—; —O—C(O)N(R₂₇)—; —N═N—; —N(R₂₇)—N(R₂₈)—;
wherein R₈ and R₉ are each independently: —H; -alkyl; -cycloalkyl; -aryl; -alkylenearyl; -alkenylenearyl; -alkynylenearyl; -heterocyclyl; -heteroaryl; -alkyleneheteroaryl; -alkenyleneheteroaryl; -alkynyleneheteroaryl; -alkyleneheterocyclyl; -alkenyleneheterocyclyl; -alkynyleneheterocyclyl; -heterocyclylalkyl; -heterocyclylaryl; -fused aryl; -fused cycloalkylaryl; -fused arylcycloalkyl; -fused cycloalkylheteroaryl; -fused heteroarylcycloalkyl; -fused heteroaryl; -fused cycloalkylheteroaryl; -fused heteroarylcycloalkyl; —S(O)₂R₃₂; —C(O)R₃₂; —C(O)NR₃₃R₃₄; —S(O)2NR₃₃R₃₄; —C(O)CR₂₉R₃₀NH[C(O)CHR₂₉NH]nR₃₁, wherein n=0, 1, 2, or 3; —C(O)CH2CR₂₉R₃₀NH[C(O)CHR₂₉NH]nR₃₁ wherein n=0, 1, 2, or 3; —C(O)CR₂₉R₃₀NH[C(O)CH2CHR₂₉NH]nR₃₁ wherein n=0, 1, 2, or 3; -alkylene-J; -alkenylene-J; -alkynylene-J; or -arylene-J; wherein R₈ and R₉ together may form a ring having the formula —(CH₂)ₒ-M-(CH₂)ₚ— bonded to the nitrogen atom to which R₈ and R₉ are attached wherein o and p are independently equal to 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—;
wherein R₃₂ is -alkyl; -alkenylenealkyl; -alkynylenealkyl; -cycloalkyl; -alkylenecycloalkyl; -aryl; -alkylenearyl; -alkenylenearyl; -alkynylenearyl; -heterocyclyl; -alkyleneheterocyclyl; -alkenyleneheterocyclyl; -alkynyleneheterocyclyl; -heteroaryl; -alkyleneheteroaryl; -alkenyleneheteroaryl; -alkynyleneheteroaryl; -fused cycloalkyl; -fused aryl; -fused heteroaryl; -fused cycloalkylaryl; -fused arylcycloalkyl; -fused heterocyclylaryl; -fused arylheterocyclyl; -fused heteroarylcycloalkyl; -fused heterocyclylheteroaryl; -fused heteroarylheterocyclyl;
and wherein R₃₃ and R₃₄ are each independently —H; -alkyl; -cycloalkyl; -aryl; -alkylenearyl; -alkenylenearyl; -alkynylenearyl; -heterocyclyl; -heteroaryl; -alkyleneheteroaryl; -alkenyleneheteroaryl; -alkynyleneheteroaryl; -alkyleneheterocyclyl; -alkenyleneheterocyclyl; -alkynyleneheterocyclyl; -heterocyclylalkyl; -heterocyclylaryl; -fused aryl; -fused cycloalkylaryl; -fused arylcycloalkyl; -fused cycloalkylheteroaryl; -fused heteroarylcycloalkyl; -fused heteroaryl; -fused cycloalkylheteroaryl; -fused heteroarylcycloalkyl;
wherein R₁₀ is —H; -alkyl; -aryl; -alkylenealkoxy; or -cycloalkyl;
wherein R₁₁ is -alkyl; -aryl; -alkylenearyl; -alkenylaryl; -heteroaryl; -alkyleneheteroaryl; -alkenylheteroaryl; -heterocyclyl; or -cycloalkyl.
wherein R₁₂ is —H; -alkyl; -cycloalkyl; -heterocyclyl; -alkyleneheterocyclyl; —alkenyleneheterocyclyl; -alkynyleneheterocyclyl; -aryl; -alkylenearyl; -alkenylenearyl; -alkynylenearyl; -heteroaryl; -alkyleneheteroaryl; -alkenylheteroaryl; -alkynyleneheteroaryl; or —NR₂₅R₂₆, where R₂₅ and R₂₆ may be taken together to form a ring having the formula —(CH₂)ₒ-M-(CH₂)ₚ— bonded to the nitrogen atom to which R₁₉ and R₂₀ are attached wherein o and p are independently equal to 1, 2, 3 or 4; M is —(CH₂)_{d}—, d=0 or 1; —O—; —S—; —S(O)—; —S(O)₂—; —C(O)—; —C(O)N(R₂₇)—; —N(R₂₇)C(O)—; —N(R₂₇)C(O)N(R₂₈)—; —N(R₂₇)S(O)₂—; —S(O)₂N(R₂₇)—; —C(O)—O—; —O—C(O)—; —N(R₂₇)S(O)₂N(R₂₈)—;
wherein R₁₃, and R₁₄ are each independently —H; -alkyl; -aryl; -heterocyclyl; -cycloalkyl; -heteroaryl; -alkylenearyl; -alkenylaryl; -alkyleneheteroaryl; -alkenylheteroaryl; -fused aryl; -fused heteroaryl; -fused cycloalkylaryl; -fused arylcycloalkyl; -fused heterocyclylaryl; -fused arylheterocyclyl; -fused heteroarylcycloalkyl; -fused heterocyclylheteroaryl; or -fused heteroarylheterocyclyl;
wherein B, C and E are each independently —(CH₂)ₙ—, n=0, 1, 2, 3, 4;
wherein F is —(CH₂)ₙ—, n=0, 1, 2, 3, 4; 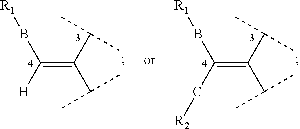
where the 3 & 4 centers may possess R or S or RS configuration when the bonds are saturated;
wherein R₁ and R₂ are each independently —H; -alkyl; -alkoxy; -alkenyl; -alkynyl; -cycloalkyl; -heterocyclyl; -aryl; -aryloxy; -alkenylenearyl; -alkenylenearyl; -alkynylenearyl; -heteroaryl; -alkyleneheteroaryl; -alkenylheteroaryl; -alkynyleneheteroaryl; -fused carbocyclic; -fused aromatic; -fused heteroaromatic; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; or -fused heteroarylheterocyclyl; alternatively, R₁ and R₂ may together form a cycloalkyl or heterocyclic ring.
wherein D is —(CH₂)ₙ—, n=0, 1, 2, 3, 4; —(CH═CH)—, n=0, 1, 2; —(CH═CR₅)—; —C(O)—; —C(O)—C(O)—; or —S(O)₂—;
wherein R₃ is —H; —C(O)OH; —C(O)OR₁₉; —C(O)NR₂₂R₂₃; —S(O)₂NHC(O)-alkyl; —S(O)₂NHC(O)-aryl; —S(O)₂NHC(O)-heteroaryl; —S(O)₂NHC(O)-alkylenearyl; —S(O)₂NHC(O)-alkyleneheteroryl; —S(O)₂NHC(O)-arylenealkyl; an acid isostere; —CHR₁₃R₁₄; —CN; —P(O)(OH)₂; —P(O)(OH)(O-alkyl); —P(O)(O-alkyl)₂; -alkyl; -cycloalkyl: -heterocyclyl; -aryl; -aryloxy; -cycloalkylaryl; -heteroaryl; -alkyleneheteroaryl; -alkenyleneheteroaryl; -alkynyleneheteroaryl; -alkylenearyl; -alkenylenearyl; -alkynylenearyl; -fused carbocyclic; -fused aromatic; -fused heteroaromatic; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; -fused heteroarylheterocyclyl;
wherein R₄ is -hydrogen; -alkyl; -alkoxy; -alkenyl; -alkynyl; -cycloalkyl; -heterocyclyl; -aryl; -aryloxy; -alkenylenearyl; -alkenylenearyl; -alkynylenearyl; -heteroaryl; -alkyleneheteroaryl; -alkenylheteroaryl; -alkynyleneheteroaryl; -fused carbocyclic; -fused aromatic; -fused heteroaromatic; -fused cycloalkylaryl; -fused arylcycloalkyl; -fused heterocyclylaryl; -fused arylheterocyclyl; -fused cycloalkylheteroaryl; -fused heteroarylcycloalkyl; -fused heterocyclylheteroaryl; or -fused heteroarylheterocyclyl.

2. The compound of claim 1 wherein the compound comprises the formula:

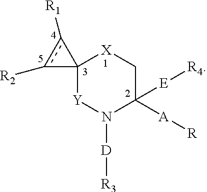

3. The compound of claim 1 wherein the compound comprises the formula: 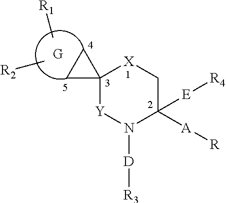
wherein G is selected from a group of ring systems consisting of -cycloalkyl, -heterocyclyl, -aryl, or -heteroaryl.

4. The compound of claim 1 wherein the compound comprises the formula: 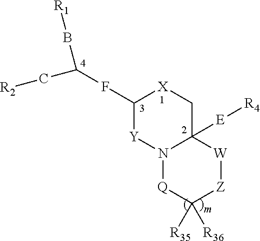
wherein W is —C(O)—; —S(O)₂—; or —(CH₂)ₙ—, n=0, 1, 2, or 3;
wherein Z is —O—; or —N—; or —S—; —S(O)—, —S(O)₂—; or —(CH₂)ₙ—, n=0, 1, 2, or 3;
wherein Q is —C(O)—; —S(O)₂— or —(CH₂)ₙ—, n=0, 1, 2, or 3; and
wherein m=1.

5. The compound of claim 1 wherein the compound comprises the formula: 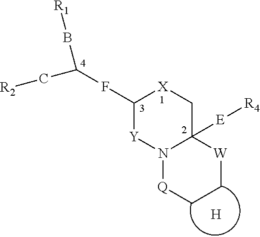
wherein W is —C(O)—; —S(O)₂—; or —(CH₂)ₙ—, n=0, 1, 2, or 3;
wherein Q is —C(O)—; —S(O)₂— or —(CH₂)ₙ—, n=0, 1, 2, or 3; and
wherein H is selected from the group of ring systems consisting of -cycloalkyl; -heterocyclyl; -aryl; -heteroaryl.

6. The compound of claim 1 wherein the compound comprises the formula: 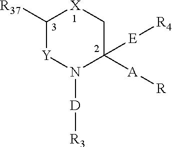
wherein R₃₇ is —H, -alkyl, -cycloalkyl, -aryl, -alkylenearyl, -alkenylenearyl, -alkynylenearyl, -alkyleneheterocyclyl, -alkenyleneheterocyclyl, or -alkynyleneheterocyclyl.

7. The compound of claim 1 wherein the compound comprises the formula: 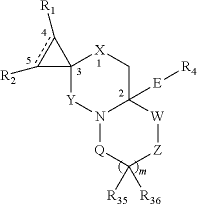
wherein W is —C(O)—; —S(O)₂—; or —(CH₂)ₙ—, n=0, 1, 2, or 3;
wherein Z is —O—; or —N—; or —S—; —S(O)—, —S(O)₂—; or —(CH₂)ₙ—, n=0, 1, 2, or 3;
wherein Q is —C(O)—; —S(O)₂— or —(CH₂)ₙ—, n=0, 1, 2, or 3; and
wherein m=1

8. The compound of claim 1 wherein the compound comprises the formula: 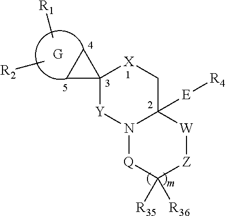
wherein W is —C(O)—; —S(O)₂—; or —(CH₂)ₙ—, n=0, 1, 2, or 3;
wherein Z is —O—; or —N—; or —S—; —S(O)—, —S(O)₂—; or —(CH₂)ₙ—, n=0, 1, 2, or 3;
wherein Q is —C(O)—; —S(O)₂— or —(CH₂)ₙ—, n=0, 1, 2, or 3;
wherein m=1; and
wherein G is selected from a group of ring systems consisting of -cycloalkyl, -heterocyclyl, -aryl, or -heteroaryl.

9. The compound of claim 1 wherein the compound comprises the formula: 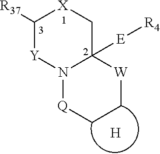
wherein W is —C(O)—; —S(O)₂—; or —(CH₂)ₙ—, n=0, 1, 2, or 3;
wherein Z is —O—; or —N—; or —S—; —S(O)—, —S(O)₂—; or —(CH₂)ₙ—, n=0, 1, 2, or 3;
wherein Q is —C(O)—; —S(O)₂— or —(CH₂)ₙ—, n=0, 1, 2, or 3;
wherein m=1; and
wherein H is selected from the group of ring systems consisting of -cycloalkyl; -heterocyclyl; -aryl; -heteroaryl.

10. The compounds of any one or more of claims 1-9 wherein the monocyclic aryl rings and fused aryl rings of the compounds comprise:
from about 1 to about 3 substituents and from about 1 to about 8 substituents, respectively;
wherein the substituents are, each independently, —H; -halo; —NR₂₂R₂₃; —NO₂; —OH; —CN; —COOR₁₉; -carbamoyl; -sulfomoyl; -alkoxy; -perhaloalkoxy; —K-alkyl; —K-cycloalkyl; —K-perhaloalkyl; —K-heterocyclyl; —K-aryl; —K-heteroaryl; —K-alkylene-heteroaryl; —K-alkylene-aryl; —K-arylene-alkyl; —K-alkylene-L-R₂₄; —K-cycloalkylene-L-R₂₄; —K-heterocyclylene-L-R₂₄; —K-arylene-L-R₂₄; —K-heteroarylene-L-R₂₄; —K-alkylene-C(O)NR₂₅R₂₆; —K-alkylene-NR₂₅R₂₆; —K-cycloalkylene-alkyl; —K-alkylene-cycloalkyl; -aryloxy-aryl; -aryloxy-alkyl; -alkoxy-alkyl; -alkoxy-aryl; -alkoxy-heteroaryl; -aryloxy-heteroaryl; 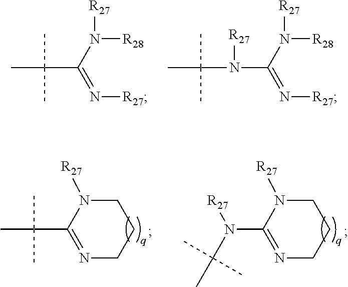
wherein q=0, 1, 2 and 3; and
wherein K and L are each independently: -alkylene-; -arylene-; -heteroarylene-; —(CH₂)_{d}—, d=0 or 1; —O—; —N(R₂₇)—; —S—; —S(O)—; —S(O)₂—; —O—S(O)—; and —O—C(O)—; —C(O)—O—; —N(R₂₇)C(O)—; —C(O)N(R₂₇)—; —N(R₂₇)C(O)N(R₂₈)—; —N(R₂₇)S(O)₂—; —S(O)₂N(R₂₇)—; —N(R₂₇)S(O)₂N(R₂₈)—; —C(O)N(R₂₇)S(O)₂—; —N(R₂₇)C(O)—O—; —O—C(O)N(R₂₇)—; —N═N—; —N(R₂₇)—N(R₂₈)—.
